SEQUENCE LISTING (12 sequences)

SEQ ID NO: 1 - protein, 1014 aa, Homo sapiens
MAESSDKLYRVEYAKSGRASCKKCSESIPKDSLRMAIMVQSPMFDGKVPHWYHFSCFWKVGHSIRHPDVEVDGFSELRWDDQQKVKKTAEAGGVTGKGQDGIGSKAEKTLGDFAAEYAKSNRSTCKGCMEKIEKGQVRLSKKMVDPEKPQLGMIDRWYHPGCFVKNREELGFRPEYSASQLKGFSLLATEDKEALKKQLPGVKSEGKRKGDEVDGVDEVAKKKSKKEKDKDSKLEKALKAQNDLIWNIKDELKKVCSTNDLKELLIFNKQQVPSGESAILDRVADGMVFGALLPCEECSGQLVFKSDAYYCTGDVTAWTKCMVKTQTPNRKEWVTPKEFREISYLKKLKVKKQDRIFPPETSASVAATPPPSTASAPAAVNSSASADKPLSNMKILTLGKLSRNKDEVKAMIEKLGGKLTGTANKASLCISTKKEVEKMNKKMEEVKEANIRVVSEDFLQDVSASTKSLQELFLAHILSPWGAEVKAEPVEVVAPRGKSGAALSKKSKGQVKEEGINKSEKRMKLTLKGGAAVDPDSGLEHSAHVLEKGGKVFSATLGLVDIVKGTNSYYKLQLLEDDKENRYWIFRSWGRVGTVIGSNKLEQMPSKEDAIEHFMKLYEEKTGNAWHSKNFTKYPKKFYPLEIDYGQDEEAVKKLTVNPGTKSKLPKPVQDLIKMIFDVESMKKAMVEYEIDLQKMPLGKLSKRQIQAAYSILSEVQQAVSQGSSDSQILDLSNRFYTLIPHDFGMKKPPLLNNADSVQAKVEMLDNLLDIEVAYSLLRGGSDDSSKDPIDVNYEKLKTDIKVVDRDSEEAEIIRKYVKNTHATTHNAYDLEVIDIFKIEREGECQRYKPFKQLHNRRLLWHGSRTTNFAGILSQGLRIAPPEAPVTGYMFGKGIYFADMVSKSANYCHTSQGDPIGLILLGEVALGNMYELKHASHISKLPKGKHSVKGLGKTTPDPSANISLDGVDVPLGTGISSGVNDTSLLYNEYIVYDIAQVNLKYLLKLKFNFKTSLW

SEQ ID NO: 2 - DNA, 3677 bp, Homo sapiens
cggtggccggtgcggcgtgttcggtggcggctctggccgctcaggcgcctgcggctgggtgagcgcacgcgaggcggcgaggcggcagcgtgtttctaggtcgtggcgtcgggcttccggagctttgccggcagctaggggaggatggcggagtcttcggataagctctatcgagtcgagtacgccaagagcgggcgcgcctcttgcaagaaatgcagcgagagcatccccaaggactcgctccggatggccatcatggtgcagtcgcccatgtttgatggaaaagtcccacactggtaccacttctcctgcttctggaaggtgggccactccatccggcaccctgacgttgaggtggatgggttctctgagcttcggtgggatgatcagcagaaagtcaagaagacagcggaagctggaggagtgacaggcaaaggccaggatggaattggtagcaaggcagagaagactctgggtgactttgcagcagagtatgccaagtccaacagaagtacgtgcaaggggtgtatggagaagatagaaaagggccaggtgcgcctgtccaagaagatggtggacccggagaagccacagctaggcatgattgaccgctggtaccatccaggctgctttgtcaagaacagggaggagctgggtttccggcccgagtacagtgcgagtcagctcaagggcttcagcctccttgctacagaggataaagaagccctgaagaagcagctcccaggagtcaagagtgaaggaaagagaaaaggcgatgaggtggatggagtggatgaagtggcgaagaagaaatctaaaaaagaaaaagacaaggatagtaagcttgaaaaagccctaaaggctcagaacgacctgatctggaacatcaaggacgagctaaagaaagtgtgttcaactaatgacctgaaggagctactcatcttcaacaagcagcaagtgccttctggggagtcggcgatcttggaccgagtagccgatggcatggtgttcggtgccctccttccctgcgaggaatgctcgggtcagctggtcttcaagagcgatgcctattactgcactggggacgtcactgcctggaccaagtgtatggtcaagacacagacacccaaccggaaggagtgggtaaccccaaaggaattccgagaaatctcttacctcaagaaattgaaggttaaaaaacaggaccgtatattccccccagaaaccagcgcctccgtggcggccacgcctccgccctccacagcctcggctcctgctgctgtgaactcctctgcttcagcagataagccattatccaacatgaagatcctgactctcgggaagctgtcccggaacaaggatgaagtgaaggccatgattgagaaactcggggggaagttgacggggacggccaacaaggcttccctgtgcatcagcaccaaaaaggaggtggaaaagatgaataagaagatggaggaagtaaaggaagccaacatccgagttgtgtctgaggacttcctccaggacgtctccgcctccaccaagagccttcaggagttgttcttagcgcacatcttgtccccttggggggcagaggtgaaggcagagcctgttgaagttgtggccccaagagggaagtcaggggctgcgctctccaaaaaaagcaagggccaggtcaaggaggaaggtatcaacaaatctgaaaagagaatgaaattaactcttaaaggaggagcagctgtggatcctgattctggactggaacactctgcgcatgtcctggagaaaggtgggaaggtcttcagtgccacccttggcctggtggacatcgttaaaggaaccaactcctactacaagctgcagcttctggaggacgacaaggaaaacaggtattggatattcaggtcctggggccgtgtgggtacggtgatcggtagcaacaaactggaacagatgccgtccaaggaggatgccattgagcacttcatgaaattatatgaagaaaaaaccgggaacgcttggcactccaaaaatttcacgaagtatcccaaaaagttctaccccctggagattgactatggccaggatgaagaggcagtgaagaagctgacagtaaatcctggcaccaagtccaagctccccaagccagttcaggacctcatcaagatgatctttgatgtggaaagtatgaagaaagccatggtggagtatgagatcgaccttcagaagatgcccttggggaagctgagcaaaaggcagatccaggccgcatactccatcctcagtgaggtccagcaggcggtgtctcagggcagcagcgactctcagatcctggatctctcaaatcgcttttacaccctgatcccccacgactttgggatgaagaagcctccgctcctgaacaatgcagacagtgtgcaggccaaggcggaaatgcttgacaacctgctggacatcgaggtggcctacagtctgctcaggggagggtctgatgatagcagcaaggatcccatcgatgtcaactatgagaagctcaaaactgacattaaggtggttgacagagattctgaagaagccgagatcatcaggaagtatgttaagaacactcatgcaaccacacacaatgcgtatgacttggaagtcatcgatatctttaagatagagcgtgaaggcgaatgccagcgttacaagccctttaagcagcttcataaccgaagattgctgtggcacgggtccaggaccaccaactttgctgggatcctgtcccagggtcttcggatagccccgcctgaagcgcccgtgacaggctacatgtttggtaaagggatctatttcgctgacatggtctccaagagtgccaactactgccatacgtctcagggagacccaataggcttaatcctgttgggagaagttgcccttggaaacatgtatgaactgaagcacgcttcacatatcagcaagttacccaagggcaagcacagtgtcaaaggtttgggcaaaactacccctgatccttcagctaacattagtctggatggtgtagacgttcctcttgggaccgggatttcatctggtgtgaatgacacctctctactatataacgagtacattgtctatgatattgctcaggtaaatctgaagtatctgctgaaactgaaattcaattttaagacctccctgtggtaattgggagaggtagccgagtcacacccggtggctctggtatgaattcacccgaagcgcttctgcaccaactcacctggccgctaagttgctgatgggtagtacctgtactaaaccacctcagaaaggattttacagaaacgtgttaaaggttttctctaacttctcaagtcccttgttttgtgttgtgtctgtggggaggggttgttttggggttgtttttgttttttcttgccaggtagataaaactgacatagagaaaaggctggagagagattctgttgcatagactagtcctatggaaaaaaccaagcttcgttagaatgtctgccttactggtttccccagggaaggaaaaatacacttccacccttttttctaagtgttcgtctttagttttgattttggaaagatgttaagcatttatttttagttaaaaataaaaactaatttcatactatttaaaaaaaaaaaaaaaaaaaaaaaaaaaa

SEQ ID NO: 3 - protein, 822 aa, Homo sapiens
MGFGSDLKNSHEAVLKLQDWELRLLETVKKFMALRIKSDKEYASTLQNLCNQVDKESTVQMNYVSNVSKSWLLMIQQTEQLSRIMKTHAEDLNSGPLHRLTMMIKDKQQVKKSYIGVHQQIEAEMIKVTKTELEKLKCSYRQLIKEMNSAKEKYKEALAKGKETEKAKERYDKATMKLHMLHNQYVLALKGAQLHQNQYYDITLPLLLDSLQKMQEEMIKALKGIFDEYSQITSLVTEEIVNVHKEIQMSVEQIDPSTEYNNFIDVHRTTAAKEQEIEFDTSLLEENENLQANEIMWNNLTAESLQVMLKTLAEELMQTQQMLLNKEEAVLELEKRIEESSETCEKKSDIVLLLSQKQALEELKQSVQQLRCTEAKFSAQKELLEQKVQENDGKEPPPVVNYEEDARSVTSMERKERLSKFESIRHSIAGIIRSPKSALGSSALSDMISISEKPLAEQDWYHGAIPRIEAQELLKKQGDFLVRESHGKPGEYVLSVYSDGQRRHFIIQYVDNMYRFEGTGFSNIPQLIDHHYTTKQVITKKSGVVLLNPIPKDKKWILSHEDVILGELLGKGNFGEVYKGTLKDKTSVAVKTCKEDLPQELKIKFLQEAKILKQYDHPNIVKLIGVCTQRQPVYIIMELVSGGDFLTFLRRKKDELKLKQLVKFSLDAAAGMLYLESKNCIHRDLAARNCLVGENNVLKISDFGMSRQEDGGVYSSSGLKQIPIKWTAPEALNYGRYSSESDVWSFGILLWETFSLGVCPYPGMTNQQAREQVERGYRMSAPQHCPEDISKIMMKCWDYKPENRPKFSELQKELTIIKRKLT

SEQ ID NO: 4 - DNA, 12134 bp, Homo sapiens
gtcacaccctcgaataatgacgcatacctatcctactgttactgatcacctagtaataatcttgtagttcacattactcatttttcaccaaattcttttggtgaaggacgcttcagaaacggccatcactgaagagcagacccgtttgggttctccacgcattctagactcccgaagagctcatgttttttggctagacctatgaccattttcgctagacttcactgcacgttttctcaagtatcttctttgtccctaatgtgtgacacctcatcatggacacgctactttagctaaggcatgaccagcaatgaacagtagtaagatatgtgctgattagaaggctcacttgtgcagtgtggaggataaccagtgccttacaaaatggggtttgggagtgacctgaagaattcacatgaagcagtgttaaaattgcaagactgggaattacggttactggaaacagtaaagaaatttatggccctgagaataaaaagtgataaagaatatgcatctactttacagaacctttgtaatcaagttgataaggaaagtactgtccaaatgaattatgtcagcaacgtatccaagtcttggctacttatgattcagcagacagaacaacttagtaggataatgaagacacatgcagaggacttgaactctggacctttacacaggctcaccatgatgattaaggacaagcagcaggtgaagaaaagttacataggtgttcatcagcagatagaggcagagatgatcaaggttaccaaaacagaattggagaagttaaaatgcagctatagacaattaataaaagaaatgaattctgccaaagagaaatataaagaagctttagctaaagggaaggaaactgaaaaggccaaggaacgatacgacaaagccacaatgaaacttcatatgttgcacaatcagtatgtattggcgttgaaaggggcacagctccatcagaatcagtattatgatatcacacttcccctgcttctggactccttacaaaagatgcaagaagaaatgataaaagcactcaaaggtatatttgatgaatacagccagataaccagtcttgtcacagaggaaatagtgaatgtccataaagagattcaaatgtcggttgaacagatagatcctagtacagaatacaataatttcatagatgttcacagaacaacggctgctaaagaacaagaaatagagtttgatacttccttactggaagaaaatgaaaatcttcaggcaaatgagatcatgtggaataacttaacagcagaaagtttgcaagtaatgttgaaaacgttagcggaagaacttatgcaaacacagcagatgcttttaaacaaggaggaggctgttttggagttagagaagagaattgaagaatcttctgaaacttgtgagaagaagtctgatattgtgcttctgctaagccaaaaacaggcacttgaagaactgaaacagtcagtccagcagctgagatgcactgaagcaaagttttcagcacagaaggaattactagagcaaaaagtgcaagaaaatgatgggaaagagccacctccagtagtaaattatgaagaagatgcacgatcagttacatctatggaaagaaaggagaggctatccaaatttgaatctattcgtcattcaattgctggaataattaggtctccaaaatctgcactgggctcttcagcactttctgatatgatctccatcagtgagaagcctttggcagaacaggactggtaccatggtgcaattcccagaatagaagctcaagaactgttaaaaaaacaaggagactttttggtgcgagagagtcatgggaaacctggtgaatatgtcctttctgtatattctgatggacagaggagacattttatcatacaatatgttgataacatgtatcgattcgagggcactgggttttcaaacattcctcaacttatagatcatcactatacaacaaaacaggtcatcactaagaaatcaggtgtagttctgctgaatcctattcctaaggacaagaaatggattctcagtcatgaagatgtcatattgggagaattactgggcaagggaaattttggtgaagtatataagggcacattaaaggataaaacttctgttgctgttaaaacatgtaaagaagatcttcctcaggaattgaaaataaaatttttacaagaagccaaaattctcaagcaatatgatcatcccaatattgtcaaacttataggagtttgcacacaaagacagcctgtctacatcattatggaactggtttcaggaggtgatttcctcacctttctgagaaggaagaaggatgaactaaaactcaaacagttagtgaaattttcattagacgctgctgctggtatgttgtatctcgagagtaaaaactgtatacacagggaccttgctgcaagaaactgcctggtaggtgaaaataatgttctgaaaatcagtgactttggaatgtctcgtcaagaggatggtggagtgtattcatcttctggcttaaagcagattcccattaaatggacagcaccggaagctcttaattatgggagatacagttcagagagtgacgtgtggagctttggcatccttctctgggagaccttcagcttaggggtttgtccgtaccctggaatgacaaatcagcaagcaagagagcaagtagaaagaggataccggatgtcagctccccagcactgtccagaggatatttccaaaatcatgatgaagtgttgggattataaacctgaaaatcgccctaagttcagtgaacttcagaaagagctcactatcatcaagagaaaactcacatagtgacaggatggcgccaaactcagccttcaggactctgtcctccagcagagtaacattattgttctcattaacaatgaatttataccacattaccttcgacagtcttctaccattattttttattaactgggtgttttaaaagtacgttccacttgtaaaaagtcaaaggcaaatttgttaaagaaataggcagtcctaccaagggctttcttagctaaccatagcaatcctaccatttcaggccattgcaaatagaaaagcacaggcttctaagtgtgtgaaggataaaagatctatcctatccttttcacaccttgtttctacttcaggcacagtttgtaggctgccatcctatgccacagaccctactccgttggtgctataaacagcattggtaatgccatgtttgcaggacatgttccaaccacagctggctttcacctctgccaaagcaggattatatttgtaaacaaacaaacagaattttatattgggaattatttggacctcctgagatttttcttttctttcttgccaggtataagcccaatgtaagaatcatcatgctcctacattatttgtaaggttgtcattttcctcctcctttgtctgggcatcagaaaacaatgcacgtaggtcgggcacggtggctcacgcctgtaatcccaggcatgtaatctcatgcctgggattacaggcgtgagccacagtgcccagccccctccctcctttgggaggctgaggcaggcggatcacctgaggtcaggagttcgagaccagcctggcttgcatggtgaaaccctgtctctaccaaaaaaaaaaaaagaaacacacacacaacgcatgaaacgtcgtcaaagttagagttgaggcagttgcttttcaaggatgtacagagagctgtgaagcaagaaacagatttgaacaggagggtgggctggagaacagagcagctaaagaatgattaacagagtaaagaagaaagtgaaaatataaaaccaccttatcagagttatgcctttctaaacatctaaacaaacaaacattcacatattctattctaaaaacacataataatacatattctattttatggtctgaatttaccagaaaggtcctaagaaaagtttcaggagttcgtagagtcctcgtaatggcaacaaaaattttgagagaaactgcaaagcacaaatccacagaaataacagcatactttcagttaagcattttcaccttagagggggcattccaaaatgtaatttcctttatttgtaccagtttttagagctgtcagaatttcatgattctagttcaccaacacattcacgtgtgtacatgcgcgtgcacacacaccaccaaggtgcaagacagatgtgaatgaagtagacagtctagaagtcaggtgaatattaatgagaaatatgacatagggacagccctcccatttctacttccaccttcattgaccattatactgccccctggacattcttaccattagctgacaggtatgcaagcagagtgctgaagatgtctcatgtgaataaaatgtaactcactcactcagttaaaactaggattctgtatcttaactcttatctagggaacttacttacttaccccatgatctgtatatagttgcctctcaggataaatgcatatatagaagatggtcatgctcagggctaaatatgttctggaagccttttataagttctcaggcctgtgtcctggtaaacattaccttagtgattatttggtctgcattcattggaagagcttagggatagaaaatgcctcctgccctctccagctgtttgctgcctgtaccatcgcacagaactcacaccagtactttagagtatgaaagataatactgaagagaaaagtgacttgtatctaacatttcaatctccttgcagatttcatacatacttaatgattagtacattcagactaaagggaaatttcttttgatggcacgtactatggtaaatcaaagcgcaaactcagaagtgctatggtattgaggcacttaccaaccttcaaaagtaagagtaaaatgggatattcctacccttttttttagtatttctaagtgaatctctgtaatattctttgtctgtttaaagaaaacagcctcccatctcatagtggcttcatgctaagctctcagcaaggagagagaatgatggttttggctttagttttatgaacaagacctaaagcttaatcctgacattctggtgttaagaatgaacacagcattgatatttcacttatccaggctgttagaatttgcagttcacatcattactctgtgcttaaatttaaaaagcttttcaaaacatcgtgacatgtgtagcatttaaattccattttattctttgtcctaaagcaggcagtggtatagttcaggattaataacttattgggagtctctcagtcatatagaatattaaatttaatatagtcacatgcattaagaaaaacttacgcaaacagtttttcactgttaattttgttgtgcttgaagcataatttattcatcccttctgtcactgataattaattattggaattatatactttaggaggctaatccatgcccaggaagcacgaacatactgttcagaatgtgcaagttgatgtagggaaaggaagattcagaaatgtacaaaatcctgtgatttgtttagtaagaaaagagttgtaaccaaaatttcttctggtccggatgtaattagtccagtcacaaatctttgtgttgttaatggattattcaagatatatatatatattcttactactgcatagttcctctcataagtccttaaatttattaaaagggggaggtaaacacaagcagtctgtgtctcactggtcactaaggggcctttatgagacagtttaggttgttcatcatttcacaaggaaagaaatttttcactgttaaattaacatttattaccaacatgtgaaacctaaaattaattttgcattccatgtaatctaaggccaaagccaacatattctttctcttctgggaaaataaaattgacaggttttgtttaaacttcagaaactgaagctttatttcagcctgtcctgccatgaggtctccagtgagaggtctccagtgaaaggtctcatgtaattgtttctcttttatctcccaaatttaatgtgggcaataaaattctcctttcgtaattcactgttttattacaaatacgatattactgaaacatcttattagcagataagtatagaccaggagcctaactgtgttagatattttcagtgcacatttcacagttgtttttcattttaggattattttgagaagaaaaaatgttttgtaattgaaatagttttaatttcctttaaaccataatttggtttttctatctatattatattcagttcagttttcccatataacttttgtgaagattggctagtaccacaaaataactatggcttcacaatactagtgttagattatgtcaagaagaaagtgttttagttacaacatacgccacccactgaggaccttgtagtgggataaacaaaaattaaaatgcaacaatccaaataaattataatgtaacaatacaaatatctgaatcataagtcacagtttttcagggttctttcagtcacaccactttataattaattctggtgagtcatcagatgtaggaatgtacctgaaaaccttacttagtatactgtgtacactgtttaatggccatgcatcttttgaagtttgatattttctgaaaaagcattgtttgcctcagccctcttctttttacgtatactccatttatcattgatattttagatgtttcttatcaacaaaattggctgccctggttagtgctgagtcatcgcaagacttgcttttgcatttaggaagccacaccctcccacaaaacagaacagtgcaaaataagcagcagagagacagacagttggaaaagacatgttttctttaggtattaatatcttaattaaatggaatcagattttgtatggaaatgagtttatattacatttccattatactaagtcagtgtgaaagtttaccatcaaaatagcgttacttcttttatgactaactctgatttgcttatacatatcagaatgatattcattaaatattttaatttcatttatttttctatgttttgattagtttcacatcaagtctgaatactatattttggaagaatccaaaaccacatatgaagatactgattttaaaagtcaggatttcatgtaccaagaatgacctacttgaaggagattttgctacactcttagagtcccaaaaattcccctttaaaaaaaatgctgtcctacagaaggtgtataaactataccttagccataattcaatttaatgaaatactaccccattaaaagccatcctcctgcctcataccgattcagaaattaaattctgaacttttagtgtaggcattaggaatcacttagccacttaataaggactgagattcttgggtggcagaaaaatcctggtgtgctagattcttagtcactttaaactgcatagaagtaatgagcataggcttcagtatatttaataaggtgctgaggcagcagattcaaatgcaaaagagggaggaaccagttattccaaggcaggatgatgcacccccacacagagtgtggaaaaattaaagtgaaacaaagatttggacagtgtcattaggaagcctgaggagaacatgctacaaggccagatcctgtcctatggaaaataaaaaagatcagcagaactaaagcaaaagcagtcatctccagtgtgtgggccttagcaaagagtaaaccaaatacctctccttgtgttaggagcactgttatattcccagctcagagatgattgtgtgctgggaaatgcttatattcattatttcccaactaatgcatttggacatatgtattccagtttttcaaagacactacccaaagcttctttacatttgcattggttatatttaatatttcttaagctaagatcactgattatccacacgttattagaaatctttggttacagtgaggcttaacctgggctaaagtctcctaaatgggactacacttagatccgccctatttcacctttggactaacgttgactaaatggctcctagagttacccacagagggagcttactatgatgagagctactgtggggaaacatagtattcaacagagaaatggaagaatacaagtagcaatgttccttgtcttcaataacatttgagggaaaggaatctagaaatcctaaagcttagtcagggtgcctaaggtaagacacaaaaagaaagagacatccagaaaggaaggttgaaaggaaatgaagggcagtaacaaagcaccaatgtaaaatgacccatagccaacaataaatgcaataacaataaaggaaactaaccctcacaaagggtttgaggagcttcctgctgtgtgtccttaaacagtgcgtgcctcttaagtctctacaacacttttttaaagggcattgaattggacattggtttaatttttacctacgctccctataatctcaggaggtaaccattatttaatcagaattacccttgctctgtttatctgtgaggcctcagacacaacggtggttcagccctctccctaagcagtagcaagctgagtaatggcacacatatgtctaggatagccagcattcaggaaaattgctgtgaacctcctgtttttaattttaatatagaaaaagtctttcaggtttgtgacacgaagacagatttagcttcccacttgtcagcaaatttgtaaaaacctcaaaacaaatgaatatttggaattcactctaagagatacaaatcctgaatttctaactatcttctcaaatattactgaggccagcaagatgcaagtgttctacaaaataatgaactgttctaagtgacagtgttgatgctggaagtggaatgttatgttttcgctcagctgtattcattcagaaagtttttcttgaacatctgccacatgaggtgcagagaatactaccaggtgctagtttttccagtatttgacttctgattactatttccttttctcatctttagtttttcaagattttgctttaccaaaatagtaaagcctttatcatcagcttatattgaataatgttgtaattggtttcaatcaaagtttctcctcaggtacttgggggcccctagccttctaaggaactcccaggcacctacttaacaaggccagctacacactcagtatgtgataagccccatgatggatgcaggttagaattcaaagacctggttggagtcctagatgtggagacaggatgatcaggtcacacttgttagatgactaacactatcagtagaagctcttgagagattttcctaacgcagcaagatttctgtgagtagaggtatcctgggaggtatcctgggaggcagcctattgacttgaccaagtaagctgatcaggtggcctcctctacccactaaagaaatgtgtaaacactagcaataattgctttatcttaaactcctggacatactcagttcttccattccactgttctattgccaataccttttgttgttttcttcacactcctcttggcagcaaatgtctgaaagtatttcaattgtgtaatgttaaggagttttttcatagcttcagaaaagagggcagcaaatatgaagccttaagttcaaaataagtcattctacctagaaatacagaccccagagcacattgcatgaaaatacctgtactctgcagttcctcaaagcagtattcttcctgaaaagccaaacaccacacctattttcctatttgctaagaatcagaataagcacgttgtaaatagtatccaaagcagattctaaaatgacatagtaagaagccagattcaaattgtaaccaaagaagacaatagaaatcccactttaccccactgtcatcagttagaacacccttgcaaaaactgtaaccacttaagcaattcatctgatcccagaagatcataccttcttttgaaagtataggacagatatcagtgggaaacgtcggcgttctgagcaacacaggataaatgtaggagggccttaaaaaataaatctcaattcatacactggagcagcaaaaaactgagcaggaaaggaaacagaatccaaagtcatttttcatatagctgttgtcaaatagtataaccttggtgtcttctttgagttgcctggacagtatttatgaaacaaaaaactaaatgccccccatttggggacggggggaggggttcagacctctaacctggattcagagccttagaggccgagagggaatctggaatctggtattactgagatcctaggtaaaagaaccagcctggcagtctttcccacctcattggtccgtgcttttatttttaaacccaaaagaaagaaagaaacacaccctcttatgtaggaatttcccttttacaaataatttgacctggtagaaataaacttgcctgcctgctcttaaatgccagacagttggaagcaaatgccgagggaaaggtgcccagagccatgcttgataggactttgaatattttctccttaattaaagtacgttgcttgtatttagactataagatgctatggaggtctatcccatgccatgccaatgtgaattgctttgcttcagtaacaatcagaagaccagtccaacagaaaataacttgtcataattccaccttagattctagacctctcatacctgcagtgtacagaatatgtacatgttccaatggaattcactatttttggctttagtgtcaaagagattggttctacaaggttcatctgatttcccataacaagtaaattttataatcctatgattctaaattcaatccccaatatagattctaagcatcaaatcaaaatcacagacaaaggggaactggtcgagaggggtcttagttatttcaaatccatgaccaaagtgtccaaagacatgaaactcttatacctgctgagcatttcactttactatacaaaatgtcagctacccagttgcatcctgtgacatgatcagactgtcaatgtggaccagtggccaggagcatatttatgggccatttctgttcatcattctttacagagcattgaggtttcccactgaaacagcttctttagtcagacgtctatagattttacataaatttacatttaaatgcattaagttagatggcccaattgagcatctgaatgaatatagtgggggttggtggtggtgcaaattctgctggctttatgttatggttttcttcgtgttttttcttggttttgtctggcttcttctggcaagtgccctaaaagactggaacactgtataaagtcatagacatagaaccatatgggaaagcccagatgaaaaaatggaagaataaaatcaagttgtcaaagttccagcaacagccctgacttcttcaggaatccaagcaaattgaaagccaagacaaaatgtacaaatggtgcccatgccattcatttgactgtgggtggccctctagtctagggctctcttagtgaatggttgtggaaatatgatttttctaagttccttccttttccttttgatagatgagtttgagatgatggagtaggagtggggccctcaggcacttctggtaaagacattccacctgcaagcagcattttgagtaaagcactgctgtggtttgccgatttatggtccatttaatgttaggctaaagcacctttaatcatttttgttgttttaagataatgtatttgtgaagtggataaacactggaaatagggtgcttcttctggaaagttcagtgtaaaacacaaacaaggctttggcgggtttatctggctttataaacaagtctgaaaaatggatgaaagctaaatatataaagcagttggttgtctatcttttatcatttttactcagatctgtatttaacacttatttatttgttagtttttacattcaaaagaaatacactttgaactttggctaacattgtaggatattttttaattgtttctacatttttaaagcatgattcatcatttttgtaaacttagatcattttttaattgtcttttcttttccaatagaccagttaccactcatgtgtctgcagaacctctttattgtattcctataataaatgtaaaatatttgtagcaaaaaaaaaaaaaaaaaa

SEQ ID NO: 5 - protein, 453 aa, Homo sapiens
MEQKMKCPHCKDQLESGFGSQSCKTCALMFSSEPSTSEVHRDQERKERLSKFESIRHSIAGIIRSPKSALGSSALSDMISISEKPLAEQDWYHGAIPRIEAQELLKKQGDFLVRESHGKPGEYVLSVYSDGQRRHFIIQYVDNMYRFEGTGFSNIPQLIDHHYTTKQVITKKSGVVLLNPIPKDKKWILSHEDVILGELLGKGNFGEVYKGTLKDKTSVAVKTCKEDLPQELKIKFLQEAKILKQYDHPNIVKLIGVCTQRQPVYIIMELVSGGDFLTFLRRKKDELKLKQLVKFSLDAAAGMLYLESKNCIHRDLAARNCLVGENNVLKISDFGMSRQEDGGVYSSSGLKQIPIKWTAPEALNYGRYSSESDVWSFGILLWETFSLGVCPYPGMTNQQAREQVERGYRMSAPQHCPEDISKIMMKCWDYKPENRPKFSELQKELTIIKRKLT

SEQ ID NO: 6 - DNA, 1420 bp, Homo sapiens
ttggtaaattgacttccttgggcccactgtcactatctcttagctaaattttgagatggagcagaaaatgaaatgtcctcattgtaaggatcagctggagtctggtttcggctcccaatcctgtaaaacatgtgctttaatgttcagctctgagccttccacatcagaagtccacagagatcaggagagaaaggagaggctatccaaatttgaatctattcgccattcaattgctggaataattaggtctccaaaatctgcactgggctcttcagcactttctgatttgatctccatcagtgagaagcctttggcagaacaggactggtaccatggtgcaattcccagaatagaagctcaagaactgttaaaaaaacaaggagactttttggtgcgagagggtcatgggaaacctggtgaacatgtcctttctgtatattctgatggacagaggagacattttatcatacaatatgttgataacatgtatcgattcgagggcactgggttttcaaacattcctcaacttatagatcatcactatacaacaaaacaggtcatcactaagaaatcaggtgtagttctgctgaatcctattcctaaggacaagaaatggattttcagtcatgaagatgtcatattgggagaattactgggcaaaggaaattttggtgaagtatataagggcacattaaaggacaaaacttctgttgctgttaaaacatgtaaagaagatcttcctcaggaattgaaaataaaatttttacaagaagccaaaattctcaagcaatatgatcatcccaatattgtcaaacttataggagtttgcacacaaagacagcctgtctacatcattatggaactggtttcaggaggtgatttcctcacctttctgagaaggaagaaggatgaactaaaactcaaacagttagtgaaattttcattggacgctgctgctggtatgttgtatctcgagagtaaaaactgtatacacagggaccttgctgcaagaaactgcctggtaggtgaaaataatgttctgaaaatcagtgactttggaatgtctcgtcaagaggatggtggagtgtattcatcttctggcttaaagcagattcccattaaatggacagcaccggaagctcttaattatgggagatacagttcagagagtgacgtgtggagctttggcatccttctctgggagaccttcagcttaggggtttgtccgtaccccggaatgacaaatcagcaagcaagagagcaagtagaaagaggataccggatgtcagctccccagcactgtccagaggatatttccaaaatcatgatgaagtgttgggattataaacctgaaaatcgccctaagttcagtgaacttcagaaagagctcactatcatcaagagaaaactcacatagtga

SEQ ID NO: 7 - protein, 374 aa, Artificial Sequence
  Residues 447-820 of human Fer
MISISEKPLAEQDWYHGAIPRIEAQELLKKQGDFLVRESHGKPGEYVLSVYSDGQRRHFIIQYVDNMYRFEGTGFSNIPQLIDHHYTTKQVITKKSGVVLLNPIPKDKKWILSHEDVILGELLGKGNFGEVYKGTLKDKTSVAVKTCKEDLPQELKIKFLQEAKILKQYDHPNIVKLIGVCTQRQPVYIIMELVSGGDFLTFLRRKKDELKLKQLVKFSLDAAAGMLYLESKNCIHRDLAARNCLVGENNVLKISDFGMSRQEDGGVYSSSGLKQIPIKWTAPEALNYGRYSSESDVWSFGILLWETFSLGVCPYPGMTNQQAREQVERGYRMSAPQHCPEDISKIMMKCWDYKPENRPKFSELQKELTIIKRK

SEQ ID NO: 8 - protein, 283 aa, Artificial Sequence
  Residues 540-822 of human Fer
KKSGVVLLNPIPKDKKWILSHEDVILGELLGKGNFGEVYKGTLKDKTSVAVKTCKEDLPQELKIKFLQEAKILKQYDHPNIVKLIGVCTQRQPVYIIMELVSGGDFLTFLRRKKDELKLKQLVKFSLDAAAGMLYLESKNCIHRDLAARNCLVGENNVLKISDFGMSRQEDGGVYSSSGLKQIPIKWTAPEALNYGRYSSESDVWSFGILLWETFSLGVCPYPGMTNQQAREQVERGYRMSAPQHCPEDISKIMMKCWDYKPENRPKFSELQKELTIIKRKLT

SEQ ID NO: 9 - DNA, 32 bp, Artificial Sequence
  Forward primer for Fer KD
gccgcgaattcgaaatcaggtgtagttctgct

SEQ ID NO: 10 - DNA, 34 bp, Artificial Sequence
  Backward primer for Fer KD
cgactgcggccgcctatgtgagttttctcttgat

SEQ ID NO: 11 - DNA, 25 bp, Artificial Sequence
  siRNA-fer
acguauccaagucuuggcuacuuau

SEQ ID NO: 12 - DNA, 25 bp, Artificial Sequence
  siRNA-fer
ggaauuacgguuacuggaaacagua

(19) United States
(12) Patent Application Publication
(10) Pub. No.: US 2018/0071280 A1
(43) Pub. Date: Mar. 15, 2018

(54) MODULATORS OF CELL DEATH PROCESSES

(71) Applicant: URIFER LTD., Raanana (IL)

(72) Inventors: Yoav ELKIS, Raanana (IL); Sally SHPUNGIN, Raanana (IL); Uri NIR, Raanana (IL)

(73) Assignee: URIFER LTD., Raanana (IL)

(21) Appl. No.: 15/553,810

(22) PCT Filed: Feb. 25, 2016
(86) PCT No.: PCT/IL2016/050221

§ 371 Date: Aug. 25, 2017

Related U.S. Application Data
(60) Provisional application No. 62/121,023, filed on Feb. 26, 2015.

(51) Int. Cl. A61K 31/496; A61K 9/107

(52) U.S. Cl. CPC A61K 31/496; A61K 9/1075

(57) ABSTRACT

Provided are compositions and methods for modulating selective cell death mechanisms. More specifically, provided are methods and compositions for inducing in a cell or in a subject in need thereof, mitochondrial damage and/or ADP ribosylation activity of PARP using an effective amount of at least one inhibitor of Fer tyrosine kinase (Fer).

### FIELD OF THE INVENTION

The present invention relates to modulation of processes of controlled cell death. More specifically, the invention relates to small molecules that modulate the Fer and FerT tyrosine kinases and uses thereof in inducing selective mitochondrial damage, autophagy, necrosis and in modulating poly (ADP-ribose) polymerase 1 (PARP1) activity in cells and in subjects in need thereof.

### PRIOR ART REFERENCES

I. WO2003017817 by Nir et al., entitled “Diagnosis, prevention, and treatment of cancer”.
II. WO2009098690 by Nir et al., entitled “Novel Fer-like protein, pharmaceutical compositions containing it and method for its use”.
III. WO2010097798 by Nir et al., entitled “6-phenyl-2-[((piperidin-4-ylmethyl)-piperazin-1yl) or piperazin 1-ylmethyl)-piperidin-1-yl)]-imidazo[2,1-b][1,3,4]thiadiazole derivatives and their use”.

### BACKGROUND REFERENCES

References considered to be relevant as background to the presently disclosed subject matter are listed below:
1. Warburg, O. (1956) On respiratory impairment in cancer cells. Science 124, 269-270
2. Pedersen, P. L. (2007) Warburg, me and Hexokinase 2: Multiple discoveries of key molecular events underlying one of cancers' most common phenotypes, the “Warburg Effect”, ie, elevated glycolysis in the presence of oxygen. Journal of Bioenergetics and Biomembranes 39, 211-222
3. Gough, D. J., Corlett, A., Schlessinger, K., Wegrzyn, J., Lamer, A. C., and Levy, D. E. (2009) Mitochondrial STAT3 supports Ras-dependent oncogenic transformation. Science 324, 1713-1716
4. Yaffe, E., Hikri, E., Elkis, Y., Cohen, O., Segal, A., Makovski, A., Varvak, A., Shpungin, S., and Nir, U. (2014) Oncogenic properties of a spermatogenic meiotic variant of Fer kinase expressed in somatic cells. Cancer research, canres. 0058.2014
5. Makovski, A., Yaffe, E., Shpungin, S., and Nir, U. (2012) Intronic promoter drives the BORIS-regulated expression of FerT in colon carcinoma cells. J Biol Chem 287, 6100-6112
6. Makovski, A., Yaffe, E., Shpungin, S., and Nir, U. (2012) Down-regulation of Fer induces ROS levels accompanied by ATM and p53 activation in colon carcinoma cells. Cell Signal 24, 1369-1374
7. Baehrecke, E. H. (2005) Autophagy: dual roles in life and death? Nature Reviews Molecular Cell Biology 6, 505-510
8. Ha, H. C., and Snyder, S. H. (1999) Poly (ADP-ribose) polymerase is a mediator of necrotic cell death by ATP depletion. Proceedings of the National Academy of Sciences 96, 13978-13982.
9. Hikri, E., Shpungin, S., Nir, U. (2009) Hsp90 and a tyrosine embedded in the Hsp90 recognition loop are required for the Fer tyrosine kinase activity. Cellular Signalling 21, 588-596.

Acknowledgement of the above references herein is not to be inferred as meaning that these are in any way relevant to the patentability of the presently disclosed subject matter.

### BACKGROUND OF THE INVENTION

The present invention stems from the aspiration to achieve efficacious cancer therapy by means of development of selective inhibitors of cancer-driving aberrant and mutated regulatory pathways, thereby leading to the elimination of malignant tumors. Novelty of this approach is based on the observations that cancer cells exhibit abnormal needs of energy generation and metabolic supply, implying the existence of reprogrammed metabolic pathways and energy generation systems in cancer cells. Indeed, cancer cells adopt mitochondrial alterations and metabolic re-programming in order to sustain their unique metabolic needs and to produce the required energy and molecules to promote tumor growth.

Specifically, while normal mammalian cells primarily utilize mitochondrial oxidative phosphorylation for ATP production, cancer cells remodel their glycolytic and mitochondrial machinery so that glycolysis is up-regulated, even under aerobic conditions which would normally inhibit glycolysis, the phenomenon termed the “Warburg effect” [1, 2]. Together with increased glutamine consumption, also characteristic of cancer cells, glucose via glycolysis provides carbon skeletons, NADPH, and ATP to build new cancer cells that persist in hypoxia. More recent studies linked between sustained aerobic glycolysis and activation of oncogenes or loss of tumor suppressors, further implying the importance of mitochondrial processes in malignant transformation [3].

A notable example of mitochondrial reprogramming in cancer cells is the intracellular kinase Fer and its spermatogenic meiotic variant, FerT, which are harnessed to the reprogrammed mitochondria in colon carcinoma cells. It was previously found by the inventors that both Fer and FerT kinases bind to complex I of the mitochondrial electron transport chain (ETC) in spermatogenic and in colon carcinoma cells, and silencing of either one of them is sufficient to impair this complex activity [4]. Strikingly, directed mitochondrial accumulation of FerT in nonmalignant NIH3T3 cells increased the ETC complex I activity, ATP production and survival, contingent upon stress conditions caused by nutrient and oxygen deprivation, and further endowed the nonmalignant cells with the ability to form tumors in vivo. Thus, recruitment of meiotic components to the cancer cell mitochondria highlights the primary role of reprogrammed mitochondria in tumorigenesis.

Previous efforts by the inventors yielded a number of new anti-cancer agents affecting specific targets, which are highly expressed in malignant tumors and which increased expression is directly linked to poor cancer prognosis, specifically colorectal, breast, pancreatic and liver cancers. More specifically, WO2003017817 entitled “Diagnosis, prevention, and treatment of cancer” related to methods and compositions involving detection and modulation of the expression of TMF/ARA160 and Fer. The TMF/ARA160 protein is expressed in spermatogenic cells in the testis and was specifically associated with Golgi-derived vesicles trafficking and E3 ubiquitin ligase activity. WO2009098690 entitled “Novel Fer-like protein, pharmaceutical compositions containing it and method for its use” focused on the FerT variant (also FerC for colorectal cancer) containing Fer SH2 and kinase domains and a novel N-terminal sequence. FerC presence was detected in colon cancer and hepatocarcinoma and its knockdown interfered with cell-cycle progression and induced apoptotic cell death, an effect that was enhanced by simultaneous knockdown of Fer [5, 6]. WO2010097798 entitled “6-phenyl-2-[((piperidin-4-ylmethyl)-piperazin-1yl) or piperazin 1-ylmethyl)-piperidin-1-yl)]-imidazo[2,1-b][1,3,4]thiadiazole derivatives and their use” related to a new class of low molecular weight synthetic compounds acting as Fer inhibitors, including among others, a compound designated as “522-0251” or a tartarate salt thereof, designated herein as 0260, which could affect the viability of cancer cells. These compounds however were far from being clinically applicable, in terms of formulations and elucidation of mechanism of action.

Thus, the present invention answers the continuous need for identification and selective targeting of new players in the processes of reprogrammed metabolic and energy generation systems, and thereby finding new ways of interfering with cancer progression. This is contingent upon reconstruction and understanding of the molecular underpinning and interaction between the natural and the synthetic modulators of these fundamental processes, and application of this knowledge for intelligent and controlled intervention into mechanisms of disorders associated with programmed cell death mechanisms, including cancer, metabolic, age related and neurodegenerative disorders.

### SUMMARY OF THE INVENTION

One of the main contributions and therefore aspects of the present invention is in providing a method for inducing in a cell at least one of, mitochondrial damage and ADP ribosylation activity of poly(ADP-ribose) polymerase 1 (PARP). This method comprises the step of contacting said cell with an effective amount of at least one inhibitor of Fer tyrosine kinase (Fer) or any vehicle, matrix, nano- or micro-particle, or composition comprising the same, thereby inducing at least one of mitochondrial damage and enhancing the activity of PARP in said cell.

It is another purpose of the present invention to provide a method for inducing in a subject at least one of, specific mitochondrial damage and ADP ribosylation activity of poly(ADP-ribose) polymerase 1 (PARP). This method involves step of administering to a subject in need thereof an effective amount of at least one inhibitor of Fer tyrosine kinase (Fer) or any vehicle, matrix, nano- or micro-particle, or composition comprising the same, thereby inducing at least one of mitochondrial damage and enhancing the activity of PARP in cells of said subject.

In yet another aspect, the present invention provides a method for inducing ADP ribosylation activity of PARP in a subject in need thereof, the method comprising the step of administering to said subject an effective amount of at least one inhibitor of Fer or any vehicle, matrix, nano- or micro-particle, or composition comprising the same, thereby enhancing the activity of PARP in said cell.

Still further aspect of the invention relates to a method for treating, preventing, inhibiting, reducing, eliminating, protecting or delaying the onset of an autophagic disease or condition in a subject by administering to said subject a therapeutically effective amount of at least one inhibitor of Fer or any vehicle, matrix, nano- or micro-particle, or composition comprising the same.

Another aspect of the present invention is in providing a micellar formulation comprising a compound or a pharmaceutically acceptable salt thereof of the following structure of Formula I:

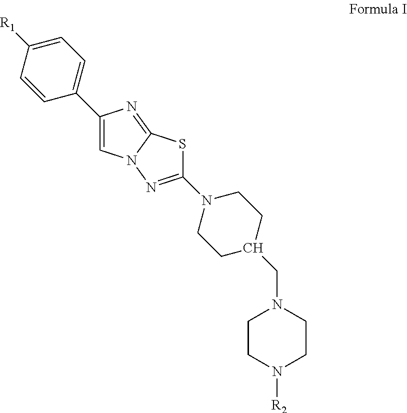

wherein R₁ and R₂ are each independently selected from hydrogen, halogen, C₁₋₆alkyl, C₂₋₆alkenyl, N—(C₁₋₆alkyl)₂ or N—(C₂₋₆alkenyl)₂, wherein the C₁₋₆alkyl and C₂₋₆alkenyl being straight or branched.

The micelles according to the present invention are formed by a method which comprises mixing a nonionic surfactant selected from polyethoxylated oil such as castor oil (e.g., Cremophor® EL), a solvent selected from C₂₋₆alcohol such as ethanol and a buffer selected from a mixture of sodium phosphate and sodium chloride or potassium chloride and potassium phosphate, such as phosphate buffered saline (PBS). The diameter of said micelle ranges between about 1 nm to about 100 nm.

Further, the present invention provides a composition comprising a micellar formulation of the compound as described above or a pharmaceutically acceptable salt thereof, said composition optionally further comprises at least one of pharmaceutically acceptable carrier/s, diluent/s and/or excipient/s.

Still further, the present invention provides a composition, specifically a pharmaceutical composition comprising said micellar formulation in an amount effective for inducing in a cell at least one of, mitochondrial damage and ADP ribosylation activity of poly(ADP-ribose) polymerase 1 (PARP).

For the purpose of relevant applications, the present invention provides a spermicidal composition comprising said micellar formulation in an amount effective for sperm cell/s of a subject and therefore compromise their motility and viability.

Still further the invention provides method for reducing at least one of motility and viability of sperm cells of a subject comprising contacting said sperm cells in said subject or in at least one further subject being in contact with said sperm cells, with a spermicidal effective amount of at least one of the Fer inhibitors of the invention.

These and further aspects and specific embodiments of the invention will become apparent by the hand of the following figures.

### BRIEF DESCRIPTION OF THE FIGURES

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIG. 1. Diameters Distribution of Micelles Containing 0260

Figure shows diameter and volume distribution of 0260 containing micelles formulated in Cremophor® EL mixed with ethanol and PBS determined by dynamic light scattering (DLS). Values presented in nm (lower rectangle VMD=volume mean diameter, ×50=median).

FIGS. 2A-2B. Cytotoxicity of 0260 Micellar and Non-Micellar Formulations

FIG. 2A shows the proportion of dead cells (%) in SW620 colon carcinoma (CC) treated for 24 hours (h) with increasing concentrations of 0260 micellar (squares) and non-micellar (diamond) formulations (left panel), including Effective Concentration 50 (EC50) values.

FIG. 2B shows the same experiment after 48 h treatment.

FIGS. 3A-3B. 0260 Effect on Tumor Progression in Mice Bearing CC Xenografts

FIG. 3A shows tumor volume (cc) in mice inoculated with CC xenografts (N=8 each group) treated four days post-inoculation, twice a day for 22 days with 0260 micellar (new) and non-micellar (old) formulations injected intra-peritonealy (IP). Experimental groups were treated with 25 mg/kg 0260 non-micellar (triangle), 50 mg/kg 0260 non-micellar (circles), 25 mg/kg 0260 micellar (dashed line) formulations. Control groups of the non-micellar formulation were administered with vehicle only (diamonds) and controls of the micellar formulation, of empty micelles (squares); *p<0.05.

FIG. 3B shows the average tumor volume in the above groups at day 22; *p<0.05.

FIGS. 4A-4B. 0260 Interference with Fer and C-Terminal Antibodies

FIG. 4A illustrates Fer enzyme functional domains, including the N-terminal Fes/CIP4 homology (FCH), coiled coil (CC), SH2 and kinase (KD) domains and antibodies directed toward various epitopes within the Fer sequence.

FIG. 4B shows immunoprecipitation (IP) of Fer from cell lysates obtained from untreated cells (control) or CC cells treated with 0260 for 16 h (0260), using antibodies directed toward the Fer N-terminal (N′), mid-part (mid) or carboxy-terminal (C′) portions, wherein C′ comprises KD (see FIG. 4A).

FIGS. 5A-5C. 0260 Docking in Fer/FerT KD

FIG. 5A is a graphic representation of the 0260 structure.

FIG. 5B shows computational model for 0260 docking in the Fer/FerT KD (light) and SH2 (dark) domains.

FIG. 5C shows the same model superimposed on hydrophobicity coloring scale (dark is hydrophilic, light—hydrophobic).

FIG. 6A-6B. shows 0260 inhibition of Fer/FerT KD autoposphorylation in vitro determined by Western blot (WB) analysis (6A) and WB quantification (6B) relating to the ratio between the phosphorylated and non-phosphorylated Fer/FerT KD.

FIG. 7. 0260 inhibition of Fer/FerT KD autophosphorylation in CC cells

Figure shows WB analysis of IP of non-activated or activated Fer from cell lysates obtained from untreated RKO CC control or treated with 2 μM 0260 for 24 h, using anti-phosphotyrosine (upper panel) and anti-SH2-Fer (lower panel) antibodies.

FIGS. 8A-8J. 0260-Induced Cell Death in Various Carcinoma Cell Lines

FIGS. 8A, 8B show the proportion of dead cells (%) in SW620 CC control or treated with increasing concentrations of 0260 for 24 h and 48 h, respectively.

FIGS. 8C, 8D show the same analysis in normal human fibroblasts (Hfb).

FIGS. 8E-8G shows the same analysis in triple negative breast carcinoma MDA-MB-231 (MDA), including 72 h time point.

FIGS. 8H-8J show the proportion of live cells in pancreatic cancer cells (PANC-1) treated with increasing concentrations of 0260 at 24 h, 48 h and 72 h time points

FIGS. 9A-9D. 0260-Indiced Cell Death in Metastatic and Advanced-Stages CC Cells

Figures show the proportion of dead cells (%) in cell lines representing various stages of cancer, HCT 116 (stage I, 9A), SW48 (stage IV, 9B), SW620 (metastatic, 9C) CC, treated with increasing concentrations of 0260 micellar formulation for 24 h (9A-9C) or with 2 μM 0260 micellar formulation for 48 h (9D).

FIGS. 10A-10N. 0260 Effect on Mitochondria in Various Carcinoma Cell Lines

Figures show transmission electron microscope (TEM) images of CC cells treated with 2 μM 0260 for 16 h (10B) and 24 h (10D, 10F) compared to untreated CC cells (treated with control vehicle) at the same time points (10A, 10C, 10E); MDA-MB-231 cells treated as above for 16 h (10H, 10J) compared to MDA-MB-231 treated with vehicle only (control) (10G, 10I); PANC-1 cells treated as above for 24 h (10L) and 72 h (10N) compared to vehicle only treated (control) PANC-1 at the same time points (10K, 10M). Arrows indicate mitochondria and arrow heads damaged cytoplasmic membranes.

FIGS. 11A-11N. 0260 Effect on Mitochondrial Membrane Potential (MMP) in Malignant Cells

FIGS. 11A-11J show fluorescent microscope images and quantitifications thereof, in two magnifications of CC cells dyed with TetraMethyl-rhodamine Ethyl Ester (TMRE) for MMP visualization and quantification, including CC cells treated with vehicle only (control) (11A, 11C) or treated with 2 μM 0260 for 12 h (11B, 11D), and MMP quantification in the above groups using ELISA (n=4); *p<0.05 (11E). MMP visualization by TMRE staining in Hfb cell system, control (11F, 11H) or treated with 2 μM 0260 for 16 h (11G, 11I), and quantification of MMP using ELISA (n=4); *p<0.05 (11J).

FIGS. 11K-11N show CC cells control (11K, 11M), or CC cells treated for 16 h with 2 μM 0260 (11L, 11N) stained with EthIII and Hoechst for nuclei, and visualized under confocal fluorescence microscope to detect necrotic cells. Size and rough texture of individual cells indicate tendency to necrosis in 0260 treated cells (11N).

FIG. 12. 0260-Induced Inhibition of the Electron Transport Chain (ETC) Complex I Activity

Figure shows CC cells treated with 2 μM 0260 compared to controls, wherein ETC complex I activity was determined at 4 h, 10 h, 16 h and 22 h time points. Significant differences between groups are indicated by esteriks (n=3).

FIGS. 13A-13D. 0260 does not Induce Apoptosis in Malignant Cells

FIG. 13A-13B show staining for AIF (Apoptosis Inducing Factor) and ATP5B ATP synthase in CC cells, control treated with vehicle only (13A) or 2 μM 0260 treated (13B)(16 h). Nuclei were visualized with Hoechst dye. AIF (dark grey) and ATP5B ATP synthase (light grey) were translocated to the nucleus

FIGS. 13C-13D show WB analysis of protein lysates from CC cells treated with vehicle only (control) or treated with 2 μM 0260 (16 h) for known apoptotic makers, cleaved CASPASE-3 (13C) and cleaved PARP-1 (13D), using anti-Caspase 3, anti-PARP-1, and anti-tubulin antibodies as controls.

FIGS. 14A-14C. 0260-Induced ATP Depletion in Malignant Cells

FIG. 14A shows quantification of ATP levels in CC cells control or treated with 2 μM 0260 (12 h), using HPLC analysis in independent experiments (n=4); *p<0.05.

FIG. 14B shows the same analysis in normal Hfb.

FIG. 14C shows ATP levels in MDA-MB-231 TN metastatic breast carcinoma cells control or treated with 2 μM 0260 (24 h), using analysis as in FIG. 14A (n=5); *p<0.05.

FIG. 15A-15B. 0260 Inhibition of Phosphorylation of AMPK Pathway Effectors in Malignant Cells

Figure shows protein lysates from CC cells vehicle only (control) or treated with 2 μM 0260 (12 h) subjected to WB analysis of phosphorylation level (pX) of selected proteins from the AMPK-mTOR signaling pathway, including AKT (15A) and RAPTOR on Ser792 and Ser 722 (15B), normalized to Tubulin.

FIGS. 16A-16I. 0260-Induced Autophagy in Malignant Cells

FIGS. 16A-16B show TEM images of CC cells treated with 2 μM 0260 for 16 h. Autophagosomes are indicated by arrows.

FIGS. 16C-16F show immunocytochemical (ICC) analysis of CC cells control vehicle only treated (16C, 16E) or treated with 2 μM 0260 (16D, 16F), using LC3 specific and secondary anti-mouse antibodies (lighter grey).

FIG. 16G shows WB analysis of CC cells vehicle only (control) or treated with 2 μM 0260 for 12 h, 16 h, 20 h, using LC3 I and -LC3II antibodies for determining LC3I/II ratio.

FIGS. 16H-16I show ICC analysis of MDA-MB-231 cells treated with vehicle only (control) (16H) or treated with 2 μM 0260 (16I), using LC3 specific antibody and secondary anti-mouse antibodies (lighter grey).

FIGS. 17A-17B. 0260 Effect on Autophagy, ATP Levels and Cell Death in Malignant Cells

FIG. 17A shows ATP levels in CC cells vehicle only (control) or treated with 2 μM 0260 (12 h) in the absence or presence of autophagy inhibitor 3-methyl adenine (3MA) in independent experiments (n=3); *p<0.05.

FIG. 17B shows the proportion of dead cells in the experiment after 16 h treatment, using a MultiTox assay kit (n=4); *p<0.05.

FIGS. 18A-18E. 0260 Effect on PARP-1 Activity in Malignant Cells

FIG. 18A shows WB analysis of CC cells vehicle only (control) or treated with 2 μM 0260 (12 h), using anti-poly-ADP-ribosylation (polyADPr) antibody compared to tubulin reference protein.

FIG. 18B shows WB analysis of protein lysates prepared from Hfb cells control or treated with 2 μM 0260, using anti-PARP-1, anti-Poly-ADPr, and tubulin as reference protein.

FIG. 18C shows IP of Fer and PARP-1 from nuclear extracts obtained from CC cells control and treated with 2 μM 0260 (12 h), using Fer N′-terminal antibodies for IP and WB and PARP1 antibodies for WB.

FIG. 18D shows WB analysis of CC cells control or treated with 2 μM 0260 in the absence or presence of the poly (ADP-ribose) polymerase (PARP-1) inhibitor, 3-amino-benzamide (3AB), compared to actin reference protein.

FIG. 18E shows the same analysis in CC cells subjected to Fer knock-down using Fer-specific siRNA (siRNA Fer) compared to controls (siRNA neg).

FIGS. 19A-19B. PARP-1 Contributes to Cytotoxicity and Autophagy Induction by 0260 in Malignant Cells

FIG. 19A shows the proportion of dead cells in CC cells treated with vehicle only (control) or treated with 2 μM 0260 in the absence or presence of PARP-1 inhibitor 3AB.

FIG. 19B shows WB analysis of the same cells using LC3 specific antibody as an autophagy marker.

FIGS. 20A-20B. The Effect of 0260 on DNA Integrity

Figures show CC cells vehicle only control (20A) or treated with 0260 (12 h, 20B) subjected to TUNEL assay to detect DNA damage (lighter grey). Nuclei detected with Hoechst stain (dark grey). 0260 has no effect on DNA integrity.

FIGS. 21A-21E. 0260-Induced Up-Regulation of Glycolysis in Malignant Cells

FIGS. 21A-21B show increased cell death in CC cells grown in DMEM and treated with increasing concentrations of 0260 (n=3) (21A), and the same cells under DMEM and 2 μM 0260 in different time points (n=4) (21B).

FIGS. 21C-21D show lactate levels which correlate to glycolysis activity (nmole/μl) secreted by CC cells treated with control vehicle or with 2 μM 0260 (12 h) in MEM or DMEM medium (21C) and normal Hfb (21D) treated as described for FIG. 21C.

FIG. 21E shows ATP content in CC cells control or treated with 2 μM 0260 (12 h) grown in DMEM (n=4).

FIGS. 22A-22D Persistence of Mitochondrial Damage in 0260 Treated Malignant Cells

Figures show TEM analysis of CC cells grown in DMEM, vehicle only (control) (22A, 22C) or treated with 2 μM 0260 (24 h) (22B, 22D). Black arrows indicate mitochondria, the white arrow points to autophagosomes. N=nucleus.

FIGS. 23A-23C. Hexokinase II Down-Regulation in 0260 Treated Malignant Cells

FIG. 23A shows WB analysis of CC cells treated with vehicle only (control) and treated with 2 μM 0260 (16 h) in MEM or DMEM medium, using specific antibodies for glycolysis enzymes, hexokinases (HK) I and II, pyruvate kinase isozymes (PKM 1/2), phosphofructokinase (PFK) and lactate dehydrogenase (LDH) compared to tubulin reference protein.

FIGS. 23B-23C show ICC analysis of CC cells vehicle only (control, 23B) or treated with 2 μM 0260 (16 h, 23C), using antibodies for HK II (darker grey) and ATPSB mitochondrial marker (lighter grey) and hoechst dye for cell nuclei.

FIGS. 24A-24B. 0260 cytotoxicity in primary and metastatic pancreatic carcinoma cells Figures show dose response curves as above in PANC-1 (primary tumor derived cells) (24A) and su.86.86 (pancreatic cancer cells derived from metastatic site-liver cells) (24B) treated with increasing concentrations of 0260 for 24 h, 48 h, 72 h. E50 values are presented (n=3).

FIGS. 25A-25B. Studies of 0260 ADME (Absorption, Distribution, Metabolism and Elimination) In Vivo in a Mouse Model

Figures show pharmacokinetic (PK) profile of 0260 in mice blood following IP administration (n=5 for each time point) (25A), and summary table for 0260 PK parameters and distribution in-vivo (25B).

FIGS. 26A-26E. The Effect of 0260 on Metastatic CC (SW620) Cells Derived Tumor Xenografts in a Mouse Model

Figures show tumors progression in mice bearing SW620 xenografts injected IP twice a day with control vehicle, 25 mg/kg 0260, or 50 mg/kg 0260 (n=8) (26A), and average tumors volume at day 22 post injection (26B), and representative images of mice bearing SW620 xenografts at day 22 post injection, when treated with vehicle only (control, 26C), 25 mg/kg 0260 (26D), and 50 mg/kg 0260 (26E).

FIGS. 27A-27E. The Effect of 0260 on Stage IV CC (SW48) Cells Derived Tumor Xenografts in a Mouse Model

Figures show tumors progression in mice bearing SW48 xenografts and injected IP twice a day with control vehicle, 25 mg/kg E260, or 50 mg/kg 0260 (n=8) (27A), and average tumors volume at day 22 post injection (27B), and representative images of mice bearing SW48 xenografts at day 22 post injection, when treated with vehicle only (control, 27C), 25 mg/kg 0260 (27D), and 50 mg/kg 0260 (27E).

FIGS. 28A-28F. Specific Effects of 0260 Treatment on Tumor Histopathology

Figures show representative images of tumor sections dissected at day 22 of treatment, at tumor center and periphery, in vehicle only (control) treated mice (28A, 28D), 25 mg/kg 0260 (29B, 28E), and 50 mg/kg 0260 (28C, 28F).

FIGS. 29A-29D. The Effect of 0260 on Weight in Mouse Model Using CC Xenographs

Figures show weight profiles of mice bearing SW620 (metastatic, 29A) or SW48 (Stage IV, 29C) xenografts and IP injected twice a day with control, 25 mg/kg 0260, or 50 mg/kg o260 (n=8), and average starting weights and end-point weights of mice bearing tumors derived from SW620 (29B) or SW48 (29D) cells at day 22 post injection.

FIGS. 30A-30I. Toxicological Studies in Mice Treated with 0260 Micellar Formulation

Figures show serum analyses of control mice and mice treated with 25 mg/kg 0260 (n=5) regarding levels of blood electrolyte (K⁺, Na⁺ and Cl⁻) (30A-30C, respectively), levels of kidney function markers including urea, total protein and creatinine (30D-30F, respectively), and levels of indices of liver function, including SGPT (ALT, alanine aminotransferase), SGOT (AST, aspartate aminotransferase and total bilirubin (30G-30I, respectively).

FIGS. 31A-31I. Histophatological Analysis of Organ Sections from Mice Treated with 0260 Micellar Formulation

FIGS. 31A-31C show representative images of histopathological analysis of heart tissues from vehicle treated (control) (312A), 25 mg/kg 0260 (31B), and 50 mg/kg 0260 (12C) treated mice, fixed, embedded in paraffin, and stained with Hematoxylin-Eosin (H&E) (n=5 in each group).

FIGS. 31D-31F show representative images of histopathological analysis of kidney tissues from vehicle treated (control) (31D), 25 mg/kg 0260 (31E), and 50 mg/kg 0260 (31F) treated mice fixed and stained as above (n=5 in each group).

FIGS. 31G-31I show representative images of histopathological analysis of liver tissues from vehicle treated (control) (31G), 25 mg/kg 0260 (31H), and 50 mg/kg 0260 (31I) treated mice fixed and stained as above (n=5 in each group).

FIGS. 32A-32B. Reduced Sperm Cell Motility Following Treatment with 0260 Micellar Formulation

Figures show quantitative analysis of images of sperm cells treated with vehicle only control (open circles) and treated with 2 μM 0260 (24 h) (black squares) regarding progressive motility (32A) and total motility (32B), including weak and not effective motility, using Computer-Aided Sperm Analysis (CASA). The proportion of hyper-activated (progressive motility) spermatozoa in each sample was determined using the SORT function of the CASA instrument. HAM was defined by curvilinear velocity (VCL)>90 μm/s, linearity (LIN) b20% and an amplitude of lateral head (ALH)>7 μm (n=3).

### DETAILED DESCRIPTION OF THE INVENTION

One of the key aspects of the present invention is the achievement of a significantly improved preparation of the 0260 compound in a micellar formulation, which proved to be significantly superior to the previous non-micellar formulation in terms homogeneity, stability and biological effects in vitro and in vivo Using a combination of two emulsification methods, i.e., Catastrophic Phase Inversion (CPI) and Phase Inversion Temperature (PIT) and specific combination of emulsifiers such as Cremophor® EL mixed with ethanol and PBS, the inventors succeeded to obtain a micro emulsion of micelles the majority of which (>95%) were loaded with the 0260 compound (EXAMPLE 1). Mathematical extrapolation of the average micelles diameter with respect to the 0260 molecular weight suggested that each micelle may incorporate between about 50 to about 500 or more molecules of the 0260 compound, specifically, between about 100 to about 250, more specifically, an average of 155.5 molecules of 0260. Furthermore, micelle containing 0260 proved to be stable in terms of droplet size and biological activity, even under prolonged storage conditions. Achievement of this superior 0260 micellar formulation has led to a series of experiments demonstrating advantageous biological novel and unexpected properties of this drug and its unique and surprising mechanism of action.

One of the main features of the 0260 compound is its capability to bind and directly interfere with the Fer kinase domain (KD) activity, as evident from computational modeling of 0260/Fer KD docking and from 0260 interference with Fer KD interaction with specific antibodies. Further, 0260 was shown to efficiently inhibit Fer KD autophosoprylation in-vitro and in-vivo (EXAMPLE 2). Owing to this unique and highly specific 0260/Fer KD interaction, which is enhanced and most probably contingent upon 0260 micellar formulation, 0260 was proved herein to be a highly selective modulator of reprogrammed metabolic pathways and energy generation systems characteristic of the malignant but not of normal cells. More specifically, colon and breast carcinoma cells treated with 0260 manifested certain phenotypes which could be related to aberrant metabolic and energy generation pathways, such as aberrant mitochondrial structure and activity, mitochondrial membrane potential (MMP) depolarization, deregulation of cellular mechanisms leading to depletion of cellular ATP, induction of autophagy and necrotic cell death, which were observed specifically in the malignant but not in normal cells subjected to the same treatment (EXAMPLES 3-8).

More specifically, the 0260 micellar formulation proved to be highly selective for killing malignant cells of various origins, colon and breast, but not normal cells (EXAMPLE 3). In terms of mitochondrial effects, 0260 was related to mitochondrial deformation and destruction and MMP depolarization (EXAMPLE 4), to down-regulation of certain mitochondrial enzymes, such as HKII and to abnormally accelerated glycolysis (EXAMPLE 7). Further, 0260 was associated with the induction of autophagy and depletion of cellular ATP (EXAMPLE 5). This excessive consumption of cellular ATP was shown to result from said accelerated autophagy as also from up-regulated PARP-1 activity (EXAMPLE 6). Collectively, the above described 0260 effects in cancer cells lead to their necrotic death.

This series of experiments have led the inventors to hypothesize that all the above-described range of 0260 effects, mitochondrial, cytoplasmic and nuclear, are mediated via 0260/Fer KD interaction and its inhibiting effect on Fer KD activity. In this sense, 0260 does not differentiate between Fer and FerT as both these proteins share the same Kinase domain. This contention as well as the mitochondrial destructive effect of this compound could explain why 0260 is effective in certain cancer cells, particularly in cells having increased mitochondrial content. Moreover, the dramatic effect of the micellar formulation of the invention on mitochondrial destruction, have led the inventors to examine the effectivity on other cells, such as spermatogenic cells. The inventors presently show that 0260/Fer KD interaction is directly responsible for regulation of PARP-1 poly-ADP ribosylation activity in cancer but not in normal cells. PARP-1 is a nuclear enzyme belonging to a family of PARP-related enzymes, which share the ability to catalyze the ADP-ribose transfer to target enzymes and thereby play an important role in various cellular processes, including modulation of NAD and ATP consumption, necrotic death onset, chromatin structure remodeling, transcription, replication, recombination and DNA repair. More specifically, the inventors have shown that apart from the above mentioned cytoplasmic and mitochondrial effects, 0260 also acts in the nucleus in interfering with PARP-1/Fer interaction and thereby in up-regulating PARP-1 activity (EXAMPLE 6).

Understanding the intracellular mechanisms underlying 0260 mode of action are important for developing novel therapeutic applications. 0260 effects proved to be highly selective for cancer cells. Further, selectivity of 0260 effect to mitochondria is contingent upon mitochondrial localization of Fer and FerT, which is particularly characteristic of cancer and spermatogenic but not normal cells [4]. Thus it was presently demonstrated that 0260 was capable of inflicting mitochondrial damage, accelerated autophagy, ATP depletion in various types of cancer cells, in other words—an energy crisis which was only partially salvaged by an accelerated glycolysis and which has ultimately led to the onset of necrotic death in these cells (EXAMPLE 6). Further, 0260 was presently shown significantly more effective cytotoxic agent in metastatic cancer and high stage tumors (EXAMPLE 8).

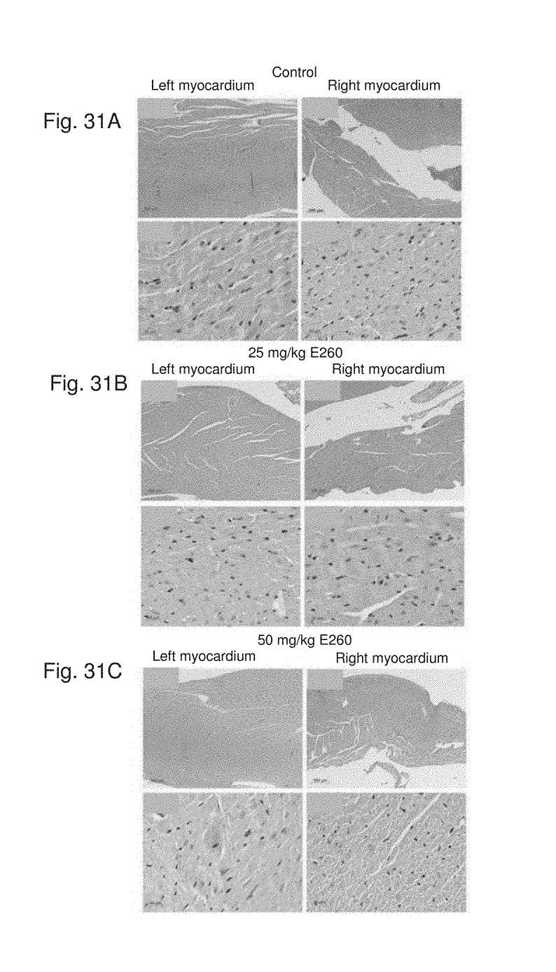

These new and surprising 0260 effects are contingent upon the conditions of 0260 delivery, i.e. 0260 micellar formulation. Finally, this new micellar formulation of 0260 proved to be toxicologically safe in mice even after prolonged treatment (22 days) with 2-daily doses of 25 mg/kg or 50 mg/kg, as evident from physiological levels of electrolytes and indices of heart, kidney and liver function in blood samples and histopathological evaluation of sections from vital organs from treated animals (FIGS. 29, 30, 31).

The present findings of unique physical and biological properties of the micellar 0260 formulation should be further interpreted to apply to pharmaceutical compositions and methods using thereof for the treatment of disorders related to 0260 cytoplasmic, mitochondrial and nuclear effects involving Fer and PARP-1.

Thus, one of the primary aspects and applications of the present invention is to provide a method for inducing in a cell at least one of, mitochondrial damage and ADP ribosylation activity of PARP. The method comprises the step of contacting the cell with an effective amount of at least one inhibitor of Fer tyrosine kinase, or any combinations thereof, thereby inducing at least one of mitochondrial damage and enhancing the activity of PARP in said cell.

Essentially, methods of the present invention enable to induce controlled and targeted cytotoxicity in specific cells. When interpreted at the cellular level, methods of the present invention are specifically intended for inducing mitochondrial damage and/or deregulation of ADP (adenosine diphosphate) ribosylation activity of PARP. More specifically, present methods by targeting Fer and inhibiting its KD (kinase domain) activity provide means for inducing selective damage in the cells distinguished by Fer expression as revealed in mitochondrial damage and enhancement of PARP activity.

In some specific embodiments, mitochondrial damage caused by the method of the invention may comprise at least one of mitochondrial membrane potential (MMP) depolarization, deformation of mitochondrial structure/s and down regulation of mitochondrial protein/s. In this context, the term mitochondrial damage refers to any changes in structural and functional properties of mitochondria. Structural changes may involve mitochondrial double-membrane organization, including the outer membrane, the inter-membrane space (also perimitochondrial space), the inner membrane, the cristae space (formed by folding of the inner membrane) and matrix (space within the inner membrane). Mitoplasts are mitochondria stripped of the outer membrane. Structural changes may involve mitochondria-associated endoplasmic reticulum (ER) membrane (MAM) associated to the outer membrane. Changes in mitochondria structure may be detected for example by TEM microscopy such as in EXAMPLE 4.

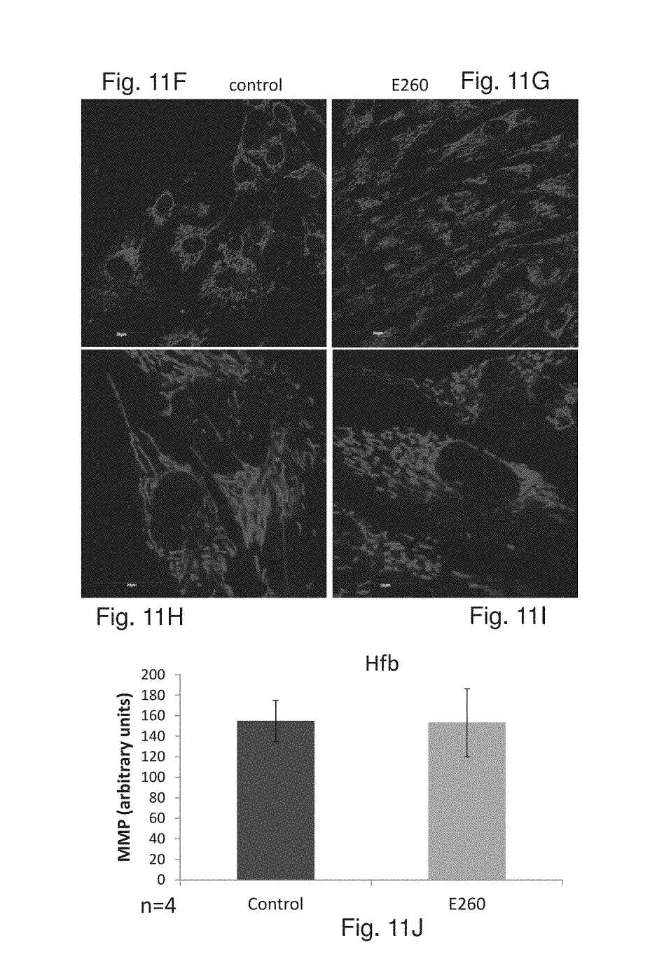

Said structural/functional changes in mitochondria may further involve changes in the composition of phospholipids and/or proteins in the mitochondrial membranes. For example, the outer mitochondrial membrane has a characteristic protein-to-phospholipid ratio of about 1:1 (by weight), similar to the plasma membrane, and a large content of integral membrane proteins (IMP, also porins or porin channels) responsible for trafficking of specific proteins into the mitochondria. As the outer membrane is freely permeable to small molecules but not to large proteins requiring active transport, the content of small molecules such as ions and sugars in the intermembrane space is similar to cytosol but the protein composition is different. Cytochrome C is one of the proteins localized to the intermembrane space. The inner membrane, in contrast, is highly enriched in protein and is also characterized by a unique phospholipid, cardiolipin. Unlike the outer membrane, the inner membrane does not contain porins and is highly impermeable unless via special membrane transporters, such as the Translocase of the Inner Membrane (TIM) complex or via Oxal. In addition, the inner membrane has a membrane potential (MMP) formed by the action of enzymes of the electron transport chain. Changes in MMP, for example, may be evaluated using fluorescence microscopy and TMRE dye as in FIG. 11.

The inner mitochondrial membrane contains the majority of proteins responsible for core mitochondrial functions, i.e. oxidative phosphorylation and production of ATP, and further specific transport proteins, and mitochondria fusion and fission proteins.

The inner membrane is compartmentalized into numerous cristae expanding its surface area and enhancing its ability to produce ATP by the ATP synthase located at the F1 particles (or oxysomes) in the inner membrane. The matrix is also important in the production of ATP due to its high content of oxidation enzymes. The matrix further contains several copies of the mitochondrial DNA genome, special mitochondrial ribosomes and tRNA, the machinery enabling autonomous replication and production of mitochondrial RNAs and proteins. MAM is another structural element that is increasingly recognized for its critical role in the ER-mitochondria calcium signaling and in the transfer of lipids between the ER and mitochondria.

Thus, structural/functional mitochondrial damage pertaining to the methods of the present invention may involve any number of changes in the above-mentioned components or activities thereof, any extent of said change or any combination or accumulation of said changes. As previously mentioned, changes in mitochondria structure may be visualized by means of fluorescent, confocal or electron microscopy using known in the art protocols and respective reagents, as presently exemplified in EXAMPLE 4, which also include evaluation of MMP. Changes in the distribution and density of specific mitochondrial proteins may also be evaluated by the same methods with the addition of immunohistochemical (ICC) analyses. Further, changes in the activity of mitochondrial enzymes, particularly those partaking in oxidative phosphorylation, may be evaluated using various molecular methods and in-vitro assays, which are well known in the art. Measurements of ATP levels resulting from the mitochondrial HKII activity using HPLC are presently exemplified in EXAMPLE 5.

Thus in specific embodiments, mitochondrial damage induced by the methods of the invention may results in at least one of MMP depolarization, deformation of mitochondrial structure/s and down regulation of mitochondrial protein/s.

Yet in other embodiments, the mitochondrial damage may result in down regulation of mitochondrial proteins, for example, Hexokinase II. It should be noted that down-regulation relates to reduction of at least one of, activity, expression, stability and appropriate localization of said mitochondrial protein.

Exemplary general methods for detecting expression and/or activity of structural proteins or enzymes include, but not limited to ELISA, Western blot, Radio-immunoassay (RIA), FACS, Immunohistochemical analysis, In situ activity assay and In vitro activity assays.

More specifically, as used herein, Enzyme linked immunosorbent assay (ELISA) involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot methods involve separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radio labeled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

In one version, the Radio-immunoassay (RIA) method involves precipitation of the desired protein (i.e., the substrate) with a specific antibody and radio labeled antibody binding protein (e.g., protein A labeled with I¹²⁵) immobilized on a precipitable carrier such as agarose beads. The number of counts in the precipitated pellet is proportional to the amount of substrate. In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Fluorescence activated cell sorting (FACS) involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

Immunohistochemical analysis as used herein relates to detection of a substrate in situ in fixed cells by substrate specific antibodies. The substrate specific antibodies may be enzyme linked or linked to fluorophores. Detection is by microscopy and subjective or automatic evaluation. If enzyme linked antibodies are employed, a colorimetric reaction may be required. It will be appreciated that immunohistochemistry is often followed by counterstaining of the cell nuclei using for example Hematoxyline or Giemsa stain.

Still further, according to In situ activity assay, a chromogenic substrate is applied on the cells containing an active enzyme and the enzyme catalyzes a reaction in which the substrate is decomposed to produce a chromogenic product visible by a light or a fluorescent microscope.

In the in vitro activity assays the activity of a particular enzyme is measured in a protein mixture extracted from the cells. The activity can be measured in a spectrophotometer well using colorimetric methods or can be measured in a non-denaturing acrylamide gel (i.e. activity gel). Following electrophoresis the gel is soaked in a solution containing a substrate and colorimetric reagents. The resulting stained band corresponds to the enzymatic activity of the protein of interest. If well calibrated and within the linear range of response, the amount of enzyme present in the sample is proportional to the amount of color produced. An enzyme standard is generally employed to improve quantitative accuracy.

Application of any one of the above methods necessitates incorporation of respective control samples to enable comparative analysis and evaluation of mitochondrial damage in terms of, for example, at least about 5%-95%, about 10%-90%, about 15%-85%, about 20%-80%, about 25%-75%, about 30%-70%, about 35%-65%, about 40%-60% or about 45%-55% change in the density or distribution of structural markers or activity of enzymes compared to corresponding control cells, i.e. non-treated (non-induced) cells which were not subjected to methods of the invention.

Specifically for mitochondrial enzymes, the damage or decrease in activity may be evaluated as at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100% change in the activity of a mitochondrial enzyme compared to corresponding control cells.

Another significant outcome of application of methods of the invention to a cell, as presently demonstrated by the inventors, is manifested in modulated PARP activity. The Poly(ADP-ribose) Polymerase (PARP) family of enzymes is characterized in that they are using β-NAD⁺ as a substrate to catalyze the transfer ADP-ribose polymer onto glutamate, aspartate or lysine residues of acceptor proteins, thereby modifying their functional properties. This protein modification is a dynamic process regulated by additional enzymes, the poly(ADP-ribose) glycohydrolase (PARG) and poly(ADP-ribose) hydrolase (ARH3), responsible for degrading the ADP-ribose polymer and thereby abolishing PARP effect. Thus, PARP enzymes take part in multiple cellular mechanisms, particularly those acting in the nucleus that are responsible for genome transcription, genome structural and biochemical integrity and DNA damage response.

When referring herein to PARP is meant the PARP family of enzymes, which for example in humans contains as many as 18 distinct proteins. PARP family members share a conserved catalytic domain that contains the “PARP signature” motif, a highly conserved sequence (100% conserved in PARP-1 among vertebrates) that forms the active site. Some PARP family members identified solely on homology, however, have not yet been shown to possess intrinsic PARP enzymatic activity. In addition to a catalytic domain, PARP family members typically contain one or more additional motifs or domains, including zinc fingers, BRCA1 C-terminus-like (BRCT) motifs, ankyrin repeats, macro domains, and WWE domains, each conferring unique properties on the particular PARP containing them.

According to a more recent nomenclature this family of proteins is referred to as ADP-ribosyl trasferases (ART) to recognize that not all family members have PARP activity and some are likely to function as a mono (ADP-ribosyl) transferases (mARTs). PARP 1-5 are bona fide PARPs containing the a conserved glutamate (Glu988 in PARP-1) that defines the PARP catalytic activity, PARPs 6-8, 10-12 and 14-16 are confirmed or putative mARTs, and PARPs 9 and 13 are lacking the key NAD⁺-binding residues and the catalytic glutamate, and thus are likely to be inactive.

Among the members of the PARP family, PARP-1 and PARP-2 are, so far, the only known members which activity is stimulated by DNA strand interruptions targeting mainly proteins involved in chromatin structure and DNA metabolism as well as PARP-1 and PARP-2 themselves. Poly(ADP-ribosyl)ation mediated by PARP-1 and PARP-2 causes chromatin decondensation around damage sites, recruitment of repair machineries, and accelerates DNA damage repair, indicating a dual role of PARP-1 and PARP-2 in the DNA damage response as DNA damage sensors and signal transducers to down-stream effectors. DNA repair pathways and cell cycle control processes have important consequences for genomic stability and tumor development.

On the basis of present findings, PARP-1 is a member of this family that is particularly relevant for methods of the present invention. When referring herein to PARP-1 (other aliases: Poly[ADP-Ribose] Synthase 1, ADP-Ribosyltransferase Diphtheria Toxin-Like 1, ADPRT1, PPOL) is meant the human PARP-1 ortholog, a 1014 amino acids protein of 113,084 Da (Enzyme Numbers (IUBMB): EC 2.4.2.3; Swiss-Prot: P09874, as also denoted herein as SEQ ID NO. 1), encoded by the hPARP-1 gene (ReFSeq mRNA: NM_001618.3, as also denoted herein as SEQ ID NO. 2) at the human 1q41-q42 chromosome. PARP-1 has a highly conserved structural and functional organization including (A) an N-terminal double zinc finger DNA-binding domain (DBD), (B) a nuclear localization signal, (C) a central automodification domain, and (D) a C-terminal catalytic domain. PARP-1 binds to a variety of DNA structures, including single- and double-strand breaks, crossovers, cruciforms, and supercoils, as well as some specific double-stranded sequences. PARP-1's basal enzymatic activity is very low, but is stimulated dramatically in the presence of a variety of allosteric activators, including damaged DNA, some undamaged DNA structures, nucleosomes, and a variety of protein-binding partners. The targets of PARP-1's enzymatic activity include PARP-1 itself, which is the primary target in vivo, core histones, the linker histone H1, and a variety of transcription-related factors that interact with PARP-1. The automodification domain of PARP-1 contains several glutamate residues that are likely targets for automodification and a BRCT motif that functions in protein-protein interactions.

Collectively, the domains and activities of PARP-1 suggest important roles in a variety of nuclear functions, including detection of DNA damage induced by oxidation, alkylation, and ionizing radiation and mediation of biochemical responses for DNA repair; regulation of chromatin structure in the presence or absence of DNA damage; regulation of transcription of specific genes as well as organization genome into discrete regulatory domains (insulator function); control of cell death pathways; and control of the mitotic mechanism required for accurate segregation of chromosomes during cell division. The molecular and cellular aspects of PARP-1 function underlie its role in many physiological and pathophysiological outcomes, including genome maintenance, carcinogenesis, aging, immunity, inflammation, and neurological function. It should be therefore appreciated that by altering PARP-1 ADP-ribosylation activity, the Fer inhibitors used by the methods of the invention, specifically the micellar formulation of 0260, may be effective in aging (including cognitive decline or any associated neurodegeneration), immunity, inflammation, and neurological function.

Thus it is contemplated that in certain embodiments, methods and compositions of the present invention when applied to a cell may provide means for modulation of poly-ADP ribosylation activity of PARP-1, specifically, modulating the addition of one or more ADP-ribose to at least one target protein. In other words, introducing into a cell an effective amount of at least one inhibitor of Fer may result in modulation of the ADP ribosylation activity of PARP-1, particularly in cells wherein Fer expression is distinguished in terms of levels and/or sub-cellular localization to mitochondria, such as for example in cancer and sperm cells.

More specifically, methods of the invention were presently demonstrated to be capable of inducing an enhancement of the poly-ADP ribosylation activity of PARP-1 in cells, specifically in colon carcinoma cells (EXAMPLE 6).

Thus in certain embodiments, application of methods of the present invention may yield enhancement, up-regulation, augmentation, elevation, increase, enlargement of PARP-1 activity, specifically, ADP-ribosylation, estimated as, for example by WB analysis and anti-poly-ADPr antibodies, at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 200%, 300%, 400%, 500%, 600%, 700%, 800%, 900% or about 1000% compared to corresponding control cells, i.e. non-treated (non-induced) cells which were not subjected to methods of the invention.

As the enhancement of PARP-1 activity also means increased consumption of ATP, it is understood that in specific embodiments, application of methods of the present invention may result in depletion of cellular ATP.

In more specific embodiments, the method of the invention may result in depletion of ATP in a cell. More specifically, such depletion may be in certain embodiments estimated as, for example by HPLC. In more specific embodiments, such depletion may result in elimination, inhibition or reduction of cellular ATP, by at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100% decrease, elimination, inhibition or reduction in ATP levels compared to corresponding control cells.

As mentioned above, PARP-1 activity is tied to a plethora of cellular signaling pathways. It is thus further contemplated that modulation and particularly enhancement of PARP-1 activity may lead to other physiological outcomes ensuing from multi-functionality of PARP-1 at the cellular level. A number of these outcomes were presently demonstrated in cancer cells, wherein application of methods of the invention resulted in augmented autophagy, increased necrosis and thereby cell death (EXAMPLE 6), which were absent in normal cells subjected with the same methods.

The terms autophagy and necrosis, as opposed to the general cell death, convey distinct pathways employed by cells to facilitate controlled cells destruction, another example of which is apoptosis. Although apoptosis and necrosis were initially thought to constitute mutually exclusive cellular states, recent findings reveal cellular contexts that require a balanced interplay between these two modes of cellular death. Several death initiator and effective molecules, signaling pathways and sub-cellular sites were identified as key mediators in both these processes, either by constituting common modules or by functioning as a switch allowing cells to decide which route to take in specific conditions. Importantly, autophagy, which is a predominantly cytoprotective process, has been linked to both, apoptosis and necrosis, serving either as pro-survival or pro-death functions.

When referring to autophagy is meant a process governing controlled cellular self-degradation that is important for balancing sources of energy at critical times in development and in response to nutrient stress. Autophagy also plays a housekeeping role in removing misfolded or aggregated proteins, clearing damaged organelles, such as mitochondria, endoplasmic reticulum and peroxisomes, as well as eliminating intracellular pathogens. Autophagy can be either non-selective or selective in the removal of specific organelles, ribosomes and protein aggregates. In addition, autophagy promotes cellular senescence and cell surface antigen presentation, protects against genome instability, giving it a key role in preventing diseases such as cancer, neurodegeneration, cardiomyopathy, diabetes, liver disease, autoimmune diseases and infections. Thus, autophagy is generally thought of as a mechanism promoting cellular survival. It should be therefore understood that in some embodiments, the methods of the invention, as well as any of the Fer inhibitors, specifically, the 0260 micellar formulation of the invention, may be applicable for any pathologies disclosed herein above.

The basic autophagy mechanism is characterized by generation of distinct cellular structures, starting from a phagophore that is likely derived from the ER and/or trans-Golgi and endosomes, which matures to an autophagosome loaded with intracellular material such as protein aggregates, organelles and ribosomes, which further fuses with lysosome to promote degradation of autophagosomal contents by lysosomal acid proteases. Lysosomal permeases and transporters export amino acids and other by-products of degradation back out to the cytoplasm, where they can be re-used for building macromolecules and for metabolism.

Of particular relevance to the present invention is a microtubule-associated soluble protein 1A/1B-light chain 3 (LC3) of approximately 17,000 Da that serves as a reliable marker of autophagy. Within the autophagosome, a cytosolic form of LC3 (LC3-0 is conjugated to phosphatidylethanolamine to form LC3-phosphatidylethanol-amine conjugate (LC3-II) attached to autophagosomal membranes. Upon autophagosome-lysosome fusion, the intra-autophagosomal components and LC3-II are degraded by lysosomal hydrolases. Thus, detecting lysosomal turnover of LC3-II and LC3-I/LC3-II ratio by immunoblotting or immunofluorescence has become a recognized method for monitoring autophagy and starvation-induced autophagic activity.

Several signaling pathways have been implicated in the regulation of autophagy. Perhaps the central signaling molecule in determining the levels of autophagy in cells is the mTOR kinase that likely mediates its effects on autophagy through inhibition of ATG1/ULK1/2 complexes (evolutionary conserved serine/threonine kinases) at the earliest stages in phagophore formation. mTOR is key to integrating metabolic, growth factor and energy signaling into levels of both autophagy, on the one hand, which is inhibited by mTOR when nutrients are plentiful and, on the other hand, to growth-promoting activities, including protein translation, that are stimulated by mTOR signaling. Low cytosolic ATP levels inhibit mTOR activity through specific negative regulators, e.g. REDD1, AMP-kinase and reduced Rheb GTPase activity, resulting in an increased autophagy. Conversely, autophagy is inhibited by up-regulated growth factor signaling acting on positive regulators of mTOR, e.g. Class I group of PI3-kinases, Akt and increased Rheb GTPase activity. Apart from depletion of cellular ATP, which is a byproduct of PARP-1 activity, activation of PARP-1 in particular has been implicated in mTOR suppression, i.e. increased autophagy.

Changes in the formation of autophagosomes and LC3-I/LC3-II turnover were presently demonstrated as one of the significant outcomes of application of methods of the invention specifically in cancer cells (EXAMPLE 6), together with mitochondrial damage and elevated PARP-1 activity. In other words, by contacting a cell with at least one Fer inhibitor, particularly a cell distinguished by expression levels or mitochondrial localization of Fer such as cancer or sperm cell, leads to a sequence of cellular events resulting in the induction of autophagy.

Thus, autophagy may be thought of as a cellular ‘recycling factory’ that also promotes energy efficiency through ATP generation and mediates damage control by removing non-functional proteins and organelles. Both autophagy and apoptosis are activated in response to metabolic stress, growth factor deprivation, limitation of nutrients and energy metabolism. In contrast, pathological necrosis has long been considered a “diametrically opposite” of apoptosis, i.e. unordered and passive cellular death in response to acute and overwhelming trauma. Morphologically, necrotic cells are characterized by swelling of organelles, such as the ER and mitochondria, rupture of the plasma membrane and cell lysis, with the nucleus, unlike in apoptosis, becoming distended and largely intact. Necrotic death is typically followed by inflammatory reactions. Mechanistically, necrosis is thought to mediate cell demise in response to damage, or in pathology, but not during normal development. Nevertheless, it became clear that necrosis can also function as an alternative programmed mode of cell death (termed necroptosis) that is very common in vivo, not only in physical traumas, but mainly in diverse forms of neurodegeneration and death inflicted by ischemia or infection. In addition, progress in the field has revealed that unlike unordered necrosis, this more physiological and programmed type of necroptotic death shares several key processes with apoptosis, including autophagy.

Thus, in certain embodiments, the invention provides methods for inducing autophagy in a cell using an effective amount of at least one inhibitor of Fer or any combination of two or more Fer inhibitors. In some specific embodiments, the Fer inhibitor of the invention, specifically, 0260 may be used.

Intracellular ATP levels have a determining role in the interplay between apoptosis and necrosis: high ATP levels typically enable a cell to undergo apoptosis, whereas low ATP levels favor necrosis. Thus, depletion of intracellular ATP levels switches the energy-requiring apoptotic cell death to necrosis. Given the central role of energy in the decision of a cell between the two modes of cell death, mitochondria are key organelles in this context. Thus, apart from cellular ATP depletion, mitochondrial damage constitutes another trigger of cell necrosis. Further, the best characterized necrosis-inducing ligand, TNFα, has been related to the activation of PARP1, thus making PARP1 act as a molecular switch between apoptosis and necroptosis.

Thus, methods of the present invention, i.e. contacting an effective amount of at least one Fer inhibitor with a cell, also provide means for inducing cellular necrosis (also necrotic cell death, or programmed necrotic cell death). According to certain embodiments, the method of the invention may be particularly relevant in cells distinguished by expression levels or mitochondrial localization of Fer. Specific cellular phenotypes indicative of necrosis may be estimated by microscopic technologies, such those presently exemplified, or on the molecular basis, for example by measuring levels of TNFα or intra-cellular ATP, to evaluate changes of at least about 5%-95%, about 10%-90%, about 15%-85%, about 20%-80%, about 25%-75%, about 30%-70%, about 35%-65%, about 40%-60%, about 45%-55%, or about 1%, 5%, 10%, 10%, 20%, 30%, 40%, 50%, 60%, 70%, 80%, 90%, or 100% of change in a necrotic phenotype compared to corresponding control cells, i.e. non-treated (non-induced) cells which were not subjected to methods of the invention.

In summary, methods of the present invention using Fer tyrosine kinase inhibitors enable to control and modulate a number of cellular phenotypes, i.e. mitochondrial damage, deregulation of PARP activity, specifically enhancement of ADP-ribosylation, ATP depletion, induced autophagy and necrosis, in specific cells wherein Fer expression is distinguished in terms of levels and/or sub-cellular localization to mitochondria, particularly the cancer and sperm cells.

Under Fer tyrosine kinase inhibitors is meant any kind of inhibitor acting specifically on the activity of Fer and Fer-related non-receptor protein-tyrosine kinases (PTK) that represent a distinct PTK subfamily, of which the only presently known members are Fer and the fps/fes oncogene are. More specifically, the fps/fes proto-oncogene, standing for fps—Fijinami poultry sarcoma and fes—feline sarcoma viruses wherein it was originally isolated, and Fer share a close structural similarity suggestive of a similar or even redundant biological roles. Activated versions of both these kinases were shown to mediate cellular transformation. Several growth factors, cytokines and immunoglobins, after engaging their receptors, were shown to induce the activation of cellular Fps/Fes and Fer. More recently, these kinases were shown to participate in the regulation of cell-cell and cell-matrix interactions, thus implicating them apart from cancer, also in the regulation of inflammation and innate immunity.

In general, the Fps/Fes and Fer PTKs share a characteristic structure consisting of an amino-terminal FCH domain, three putative coiled-coil domains, a central SH2 domain and a carboxy-terminal kinase (KD) domain. Similar amino-terminal FCH domain was found in numerous proteins implicated in the regulation of cytoskeletal rearrangements, vesicular transport and endocytosis. The coiled-coil domains seem to be responsible for the Fer and Fps/Fes ability to form oligomers, considered a key distinguishing feature of these two PTKs, homotrimers for Fer and pentamer or higher-order oligomers for Fps/Fes. The Fps/Fes and Fer oligomerization is thought to mediate inter-conversion between two conformational states, inactive monomers or active oligomers. The SH2 domain, a prototypic module of a large family of proteins, is thought to mediate protein-protein interactions, in the case of Fps/Fes and Fer these are phosphotyrosine-dependent interactions with putative substrates and regulators. Substrates for Fps/Fes and Fer tyrosine kinase activity suggested from biochemical studies include BCR (serine/threonine kinase), p120RasGAP (GTPase activating protein), STAT3 and STAT5A (signal transducers and activators of transcription), cortactin (cortical actin binding protein), certain catenins (proteins found in complexes with cadherin cell adhesion molecules) and SHP-2 (tyrosine phosphatase Shp2, also PTPN11). Further, Fps/Fes and Fer were found to be substrates of other PTKs making them inducible by various growth factors, hormones and cytokines. Among known upstream regulators of Fps/Fes and Fer are Platelet-Derived Growth Factor (PDGF), Granulocyte-Macrophage-Colony Stimulating Factor (GM-CSF), Epidermal Growth Factor (EGF) and a number of interleukins, specifically IL-3, IL-4, IL-5, IL-6 and IL-11, and erythropoietins.

Of particular relevance to the present context is the human Fer protein (other aliases: Proto-Oncogene c-Fer, Tyrosine Kinase 3 (TYK3), Phosphoprotein NCP94 (p94-Fer), Fujinami Poultry Sarcoma/Feline Sarcoma-related protein Fer, Feline Encephalitis virus-related kinase Fer), a tyrosine kinase protein of 822 amino acids of 94,638 Da, (Enzyme Numbers (IUBMB): EC 2.7.10; Swiss-Prot: P16591, as also denoted by SEQ ID NO. 3) encoded by the hFer gene (ReFSeq mRNA: NM 005246, as also denoted by SEQ ID NO. 4) at the human 5q21.3 chromosome. Of further relevance is the truncated hFer variant (designated as FerT), a 453 amino acids protein of 51,383 Da, containing Fer carboxy-terminal KD and considered to be the smallest known PTK. FerT is a tissue specific alternative splicing product of Fer transcript (NM_001037997, NP_001033086, as also denoted by SEQ ID NO. 5, encoded by the nucleic acid sequence denoted by SEQ ID NO. 6) that under normal conditions is preferentially found in the testis, but may also be present in certain cancer cells such as in colon carcinoma in metastases derived from colorectal tumors.

It should be therefore understood that when referring to Fer inhibitor, the methods of the invention encompass the use of any compound that may affect at least one of, the activity, expression, stability and cellular localization of at least one of Fer, FerT, FerT, Fps/Fes or any analogue or ortholog thereof. Such inhibitors may include for example specific siRNA, miRNAs, specific inhibitory peptides or any small molecule.

Most importantly, as presently demonstrated by the inventors, Fer and FerT also act as PTKs in mitochondria and inhibition of this activity by Fer specific PTK inhibitors (EXAMPLE 2) leads to mitochondrial structural and functional damage and to ATP depletion (EXAMPLE 5). It was further demonstrated that Fer and FerT are physically and functionally interacting with PARP-1 and that Fer specific PTK inhibitors may disrupt this interaction thereby leading to up-regulation of PARP-1 activity and further ATP consumption (EXAMPLES 5 and 6). Still further, it was demonstrated that the above effects of Fer specific PTK inhibitors were significantly more pronounced in cancer than in normal cells, suggesting that elevated expression of Fer and/or FerT and most likely, their mitochondrial localization in these cells may be responsible for said effects.

More specifically, the Fer inhibitors applicable in the methods of the invention may be any compound that inhibit, reduce, decrease, attenuates, eliminates or change at least one of, the activity, expression, stability and cellular localization of Fer. Thus it is presently contemplated that by inhibiting either Fer or FerT or Fps/Fes tyrosine kinase activity using Fer inhibitors it would be possible to induce controlled and targeted damage in mitochondria and PARP activity in specific cells. It is further contemplated that for the purpose of methods of the present invention it would be sufficient to inhibit, reduce, eliminate, attenuate, decrease, at least one of Fer and any one of its natural analogs (i.e. FerT, Fps/Fes) by at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100% compared to a control (untreated) cells not subjected to methods of the present invention.

More specifically, methods of the present invention involve the step of contacting a cell with an effective amount of at least one inhibitor of Fer or its natural analogs (i.e. FerT, Fps/Fes). The term “contacting” means to bring, put, incubate or mix together. As such, a first item is contacted with a second item when the two items are brought or put together, e.g., by touching them to each other or combining them. In the context of the present invention, the term “contacting” includes all measures, conditions or steps, which allow interaction between the Fer inhibitors of the invention and the target cells to be modulated.

There are a number of candidate Fer inhibitors that can be applicable to methods of the present invention, the majority of them, unlike the micellar 0260 formulation, do not target Fer specifically to but all receptor PTKs and/or serine/threonine kinases. Such inhibitors may include Staurosporine (also AM-2282, STS, CGP 41251) a potent, cell permeable PTK C (PKC) inhibitor, an antibiotic and a natural product originally isolated from the bacterium Streptomyces staurosporeus staurosporine manufactured by several Biotech companies; K252a (Fermentek Biotechnology) is a staurosporine analogue isolated from Nocardiopsis soil fungi; SB 218078 (CAS 135897-06-2) is an inhibitor of checkpoint kinase 1 (CHK1) serine/threonine PTK; and further to a lesser extent may include Gö 6976, a very potent PKC inhibitor; SU11652, a multitargeting receptor PTK inhibitor; Syk inhibitor, an imidazo-pyrimidine inhibitor of spleen PTK (SYK); Cdk1/2 Inhibitor III (CAS 443798-55-8), an ATP-competitive inhibitor of Cdk1/cyclin B and Cdk2/cyclin A; Midostaurin (PKC412) is a multi-target PTK inhibitor; a cell-permeable indirubin derivative (IDR) inhibiting Src-STAT3 signaling; and Sunitinib (marketed as Sutent by Pfizer and previously known as SU11248) is an oral, small-molecule, multi-targeted receptor PTK inhibitor. Advantages of the presently used Fer inhibitors and of 6-(4-isopropyl-phenyl)-2-{4-[(4-methyl-piperazin-1-yl)methyl]piperidin-1-yl}imidazo[2,1-b][1,3,4]thiadiazole, and specifically of tartarate salt thereof, designated herein as 0260, particularly in its micellar formulation, will be detailed further below.

The term an effective amount (also a sufficient amount), as used herein, means an amount necessary to achieve a desirable effect, in this case, reducing, elimination or inhibiting the kinase activity of Fer, inducing mitochondrial damage and deregulation of PARP-1 activity, specifically enhancement of its ADP-ribosylation activity, in specific cells. It may also expressed in terms of a physiologically effective amount of a bioactive agent, which is the amount of an active agent, in this case an inhibitor of Fer, which is present in a composition in order to achieve a desired level of active agent at the site of action being it a cell, an organ or a blood stream of an organism to give an anticipated physiological response when such composition is administered. Further details on compositions and formulations of the bioactive agent that are particularly relevant to the present invention will be provided in the course of this description of the invention. At this stage, however, it should be noted that the precise effective amount of a bioactive agent may depend upon numerous factors, e.g. the type of Fer inhibitor, physical characteristics and activity of the composition comprising thereof, the delivery method and delivery dose, all of which can be determined using standard clinical procedures for determining appropriate amounts and timing of administration of the bioactive agent of the invention. It is understood that an effective amount can be the result of empirical and/or individualized (case-by-case) determination.

Further in this connection, when referring herein to the step of contacting a cell with an effective amount of Fer inhibitor, includes application of any method facilitating the permeability and retention of said inhibitor to achieve the desired effect. One example of such methods is the presently used 0260 micellar formulation.

Thus, it is another embodiment of the present invention to provide an inhibitor of Fer that can be implemented in the above discussed methods. This Fer inhibitor is characterized in having the following general structure of formula I:

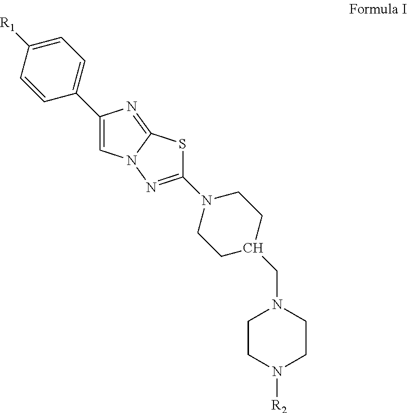

wherein R₁ and R₂ are each independently selected from hydrogen, halogen, C₁₋₆alkyl, C₂₋₆alkenyl, N—(C₁₋₆alkyl)₂, or N—(C₂₋₆alkenyl)₂, and wherein the C₁₋₆alkyl and C₂₋₆alkenyl being straight or branched. This Fer inhibitor may by a compound of the general structure of Formula I or a pharmaceutically acceptable salt thereof or any vehicle, matrix, nano- or micro-particle, or composition comprising the same.

Further, for the purpose of the present invention R₁ may be represented by hydrogen, methyl, ethyl, propyl, isopropyl, N-isopropyl, butyl, sec-butyl, tert-butyl, N-butyl, N-sec-butyl, N-tert-butyl, F, Cl, Br, I; and R₂ may be represented by hydrogen, methyl, ethyl, propyl, isopropyl, butyl.

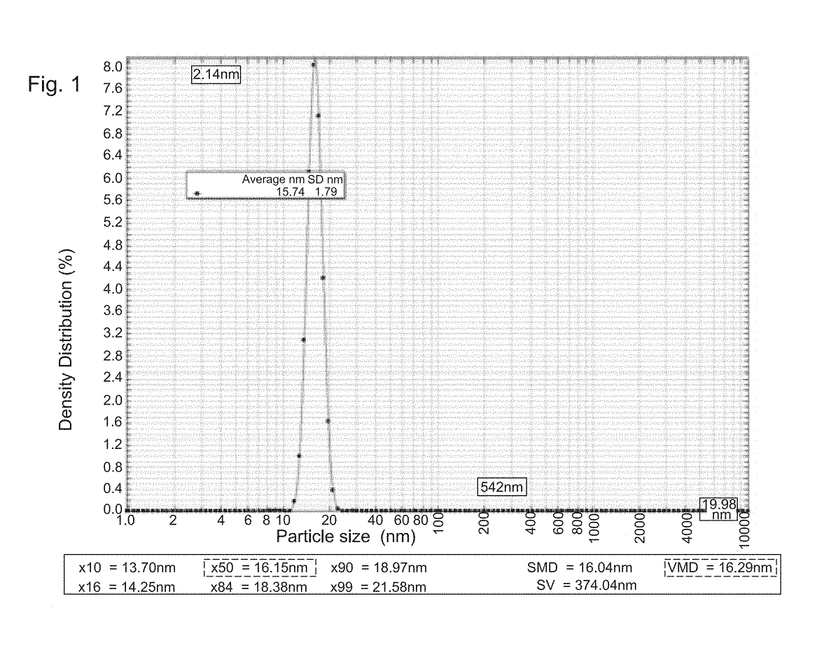

A specific example of Fer inhibitor of the invention is a compound of the above mentioned formula I wherein R₁ is isopropyl and R₂ is CH₃ referred to herein as compound “522-0251” or as compound “Z522-0251” in WO 2010/097798. It is noted that the general structure of compound “Z522-0251” is presented in WO 2010/097798 as general formula (I) (See page 2 therein). In said formula (I) of WO 2010/097798 groups R₁, R₂ and R₃ are provided; R₁ of WO 2010/097798 corresponds to R₁ in the present application; R₂ of WO 2010/097798 corresponds to H in the present application; and R₃ of WO 2010/097798 corresponds to R₂ of the present application. The specific structure of “Z522-0251” in WO 2010/097798 is depicted inter-alia in FIG. 1 thereof. The Fer inhibitor according to the invention may be also referred to as 6-(4-isopropyl-phenyl)-2-{4-[(4-methyl-piperazin-1-yl)methyl]piperidin-1-yl}imidazo[2,1-b][1,3,4]thiadiazole of the following formula:

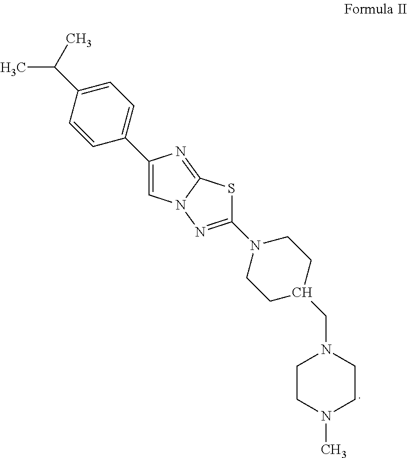 [SMILES: CC(C)c1ccc(-c2cn3nc(N4CCC(CN5CCN(C)CC5)CC4)sc3n2)cc1]

Thus, in specific embodiments, methods of the invention may use the compound 6-(4-isopropyl-phenyl)-2-{4-[(4-methyl-piperazin-1-yl)methyl]piperidin-1-yl}-imidazo[2,1-b][1,3,4]thiadiazole, a pharmaceutically acceptable salt thereof, or any vehicle, matrix, nano- or micro-particle, or composition comprising the same.

In yet further embodiments, the pharmaceutically acceptable salt of this compound is the tartarate salt of 6-(4-isopropyl-phenyl)-2-{4-[(4-methyl-piperazin-1-yl)methyl]piperidin-1-yl}-imidazo [2,1-b][1,3,4]thiadiazole, designated herein as “0260”, “compound 260” or as “E260”. Tartarate refers to the tartrate dianion is O⁻ OC—CH(OH)—CH(OH)—COO⁻ or C₄H₄O₆²⁻.

It should be noted that the compound 260 was presently demonstrated as particularly advantageous in achieving the desired effects, as detailed above.

As noted herein before, the Fer inhibitors used by the methods of the invention may be formulated in any vehicle, matrix, nano- or micro-particle, or composition. Of particular relevance are formulations of the inhibitors of the invention adapted for use as a nano- or micro-particles. Nanoscale drug delivery systems using micellar formulations, liposomes and nanoparticles are emerging technologies for the rational drug delivery, which offers improved pharmacokinetic properties, controlled and sustained release of drugs and, more importantly, lower systemic toxicity. A particularly desired solution allows for externally triggered release of encapsulated compounds. Externally controlled release can be accomplished if drug delivery vehicles, such as micelles, liposomes or polyelectrolyte multilayer capsules, incorporate nanoparticle (NP) actuators.

It should be therefore understood that the invention further encompasses the use of various nanostructures, including micellar formulations, liposomes, polymers, dendrimers, silicon or carbon materials, polymeric nanoparticles and magnetic nanoparticles, as carriers in drug delivery systems.

Still further, in specific embodiments, methods of the invention may use a micellar formulation of E260, which was presently demonstrated as particularly advantageous in achieving the desired effects, e.g. mitochondrial damage, modulation of PARP activity and induction of autophagy and necrosis in cancer cells.

The phrase “micellar formulation of E260”, “micellar 0260 formulation” or any lingual variations thereof are interchangeable and are to be envisages as formulations in which compound 0260 is incorporated into aggregates of surfactant molecules dispersed in a liquid colloid. A typical micelle in aqueous solution forms an aggregate with the hydrophilic “head” regions in contact with surrounding solvent, sequestering the hydrophobic single-tail regions in the micelle centre. It should be appreciated that in certain embodiments, the micellar formulations of the invention, specifically, the E260 micellar formulations of the invention may be oil in water micellar formulations, or “o/w micelles”. The term “o/w micelles” as used herein refer to a type of micelle also known as a normal-phase micelle (oil-in-water micelle), where the hydrophilic “head” extending out and the tails in the center. For example, micellar 0260 formulation can be achieved in the presence of Cremophor® EL mixed with ethanol and PBS with an aid of various emulsification methods.

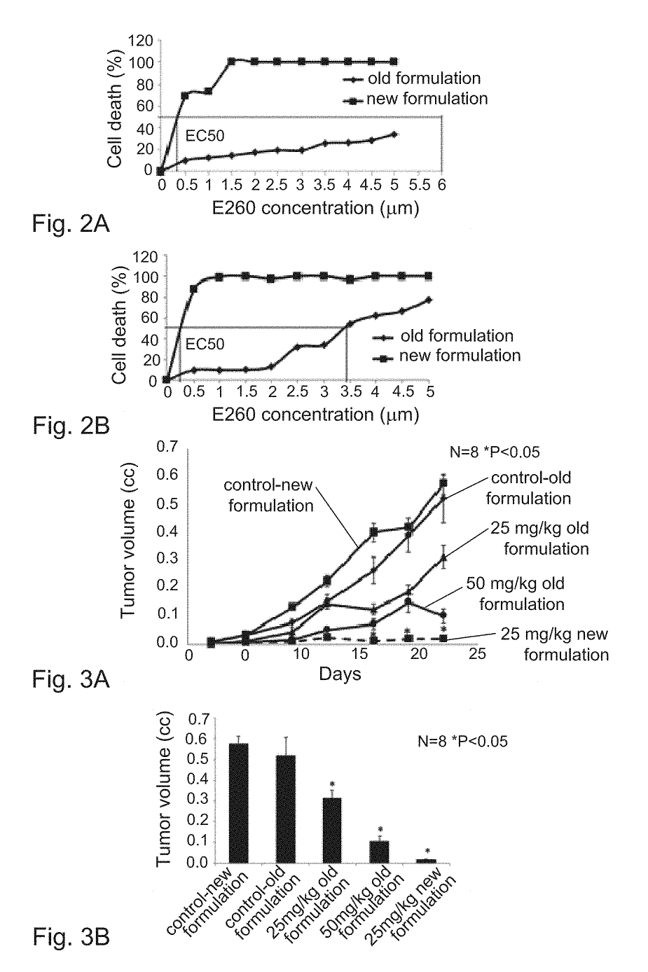

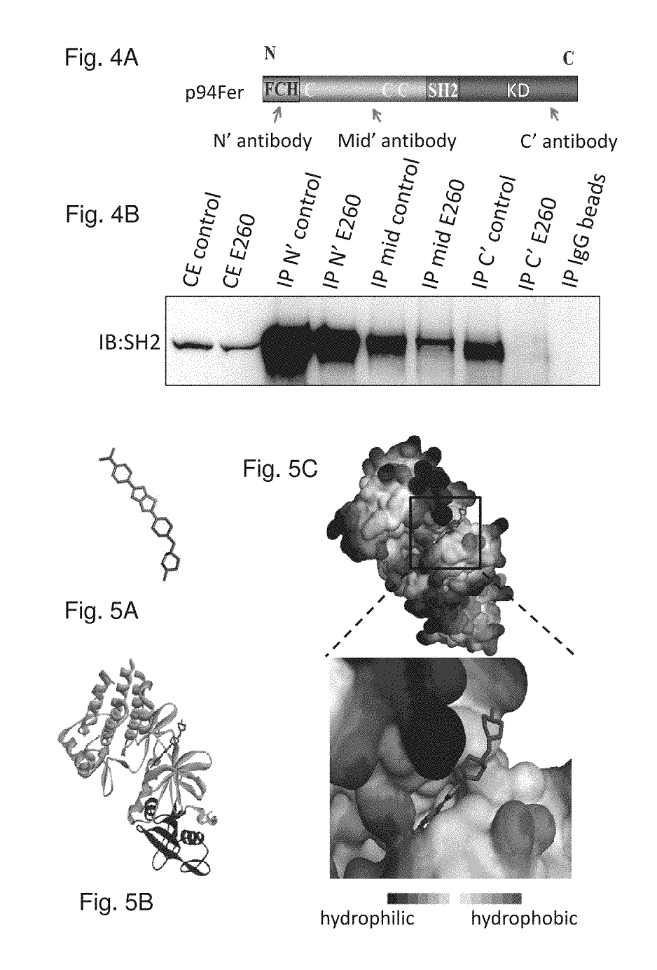

For the purpose of specific embodiments of the invention, 0260 can be incorporated into micelles formed in the presence of various proportions of Cremophor® EL (1-25%) mixed with ethanol (1-25%) and PBS, using a combination of CPI and PIT emulsification methods (EXAMPLE 1). These conditions were presently demonstrated as especially advantageous for achieving maximal micelle loading (>95%) with the 260 compound (FIG. 1), which was also reflected in biological efficacy of the micellar 0260 compared to non-micellar formulation (FIGS. 2A-2B and 3A-3B).

Thus, it is contemplated that methods of the present invention, especially those using the above-mentioned Fer inhibitors and the 0260 micellar formulation in particular, may be applicable to clinical purposes.

More specifically, methods of the present invention according to the above may be applicable to induce autophagy in cells of a subject affected with an autophagic diseases or condition. Under an autophagic disease or condition is meant a human autophagy-related disease/disorder or a clinical condition ensuing from deregulated or defective autophagy.

The role of autophagy in adaptive responses to starvation and other forms of stress and cellular homeostasis was previously discussed. In the recent years, genetic studies of autophagy-related (ATG) genes in various model organisms, including mammals, revealed that autophagy plays critical roles in cellular differentiation and development and further related between dysregulated autophagy and various kinds of disease-like phenotypes including cancer, neurodegenerative diseases, infectious diseases, and metabolic diseases.

More specifically, genetic polymorphisms in the ATGS gene were associated with asthma and enhanced risk of systemic lupus erythematosus. Crohn's disease was related to a number of mutation and polymorphisms in ATG and ATG-related genes, including ATG16L1, the Immunity-Related GTPase family M protein (IRGM), and the Nucleotide-binding Oligomerization Domain-containing protein 2 also Caspase Recruitment Domain-containing protein 15 (NOD2/CARD15). Most, notably, genes related to autosomal recessive or sporadic Parkinson's disease, i.e. Parkins PINK1, PINK2, PINK6, were implicated in the regulation autophagy. Deletions in Beclin 1 gene (BECN1, a mammalian ortholog of ATG6) were related to the risk and prognosis of human breast, ovarian, prostate, and colorectal cancers, and deletions in the apoptosis/autophagy factor E124/PIG8—to the early breast cancer in particular. Certain neurodegenerative and neuroligic disorders were related to several core components of autophagy machinery, specifically the WD repeat domain 45 (WDR45/WIPI4) gene and Zinc Finger FYVE domain-containing protein 26 (ZFYVE26/SPG15) and Ectopic P-Granules Autophagy Protein 5 Homolog (EPGS).

It is thus conceivable that human pathologies associated with accumulation of misfolded and/or ubiquitinated protein aggregates with a perturbation of autophagy may benefit from modulation and particularly enhancement of autophagy afforded by methods of the present invention. Notable examples of such pathologies are neurodegenerative disorders, such as in Parkinson's, Alzheimer's and Huntington's diseases. Further discussion on human disorders to which methods of the present invention may be applicable is presented below.

Methods of the present invention may be of particular relevance to induce autophagy and controlled cell death in cancer or sperm cells of a subject. Specifically, the cancer and sperm cells were presently demonstrated as the most sensitive to the effect of 0260 (EXAMPLES 3, 8 for cancer and EXAMPLE 10 for sperm cells). This is because in these cells Fer and/or FerT are over-expressed and/or located to mitochondria, thus making them liable to the effect of Fer tyrosine kinase inhibitor.

In the context of cancer, methods of the present invention are particularly applicable to induce autophagy and controlled cell death in metastatic cancer cells, known to be significantly distinguished with respect to Fer and/or FerT expression and/or mitochondrial localization. Significant outcomes of application of methods of the invention to metastatic cancer cells of various stages and origins, i.e. colon and breast metastatic cancers, were presently demonstrated (EXAMPLES 3, 8).

In specific embodiments, methods of the present invention provide means for inducing ADP ribosylation activity of PARP-1 in cells of a subject to control genome instability, inflammatory response, cellular energetics and death pathways. As PARP-1 can act in both in a pro- and anti-tumor manner depending on the context, it important to consider global effects of PARP-1 in determining when and how to best use PARP inhibitors or activators in anti-cancer therapy.

It is this another aspect of the present invention to provide a method for inducing in a subject at least one of, specific mitochondrial damage and ADP ribosylation activity of PARP, the method comprising the step of administering to a subject in need thereof an effective amount of at least one inhibitor of Fer tyrosine kinase (Fer), thereby inducing at least one of mitochondrial damage and enhancing the activity of PARP in cells of said subject.

It should be emphasized that unlike with other methods of treatment, such as those using psychotropic drugs, analgesics and many others, wherein mitochondrial damage constitutes an unavoidable adverse effect, for methods of the present invention mitochondrial damage constitutes one of the primary goals. This because methods of the present invention are targeting Fer/FerT which makes them highly selective in excreting damaging and cytotoxic effects in particular cell types wherein Fer/FerT is over-expressed and/or localized to mitochondria.

Thus in specific embodiments, said mitochondrial damage comprises at least one of mitochondrial membrane potential (MMP) depolarization, deformation of mitochondrial structure/s and down regulation of mitochondrial protein/s in cells of a subject subjected to methods of the invention.

As shown in EXAMPLE 5, the Fer inhibitor of the invention, 0260, led to phosphorylation and activation of the metabolic sensor adenosine-mono-phosphate protein kinase (AMPK). This was accompanied by the inhibitory phosphorylation of the AMPK down-stream effector-Raptor on serines 792 and 722 and by a decrease in the activating phosphorylation of Akt. Phosphorylation of Raptor leads to its dissociation from the mTOR complex, thereby leading to inactivation of mTOR kinase activity regardless of its phosphorylation state. Deactivation of mTOR relieves its inhibitory effect on autophagy, thereby enabling the activation of this process. It should be therefore appreciated, that the invention further encompasses methods for decreasing the activating phosphorylation of Akt in a cell.

Yet in further embodiments, said mitochondrial damage may be manifested in down regulation of mitochondrial proteins, for example, Hexokinase II in cells of a subject subjected to methods of the invention.

In still further embodiments, methods of the invention may be applicable to induce autophagy in a subject, said method comprising the step of administering to a subject suffering from autophagic disease or condition an effective amount of at least one inhibitor of Fer.

Relevance of methods of the present invention to cancer and neurodegenerative disorders was previous discussed in terms of clinical conditions which could benefit from enhanced autophagy. Among neurodegenerative disorders, methods of the present invention may be particularly applicable to Alzheimer's, Parkinson's, Huntington's, Creutzfeldt-Jakob's diseases, which are associated with accumulation of misfolded and/or ubiquitinated protein aggregates with a perturbation of autophagy.

Further, accumulation of misfolded proteins in aggregates has been observed in airways of patients with cystic fibrosis (CF). Furthermore, deficiency of the Cystic Fibtosis Transmemebrane Conductance Regulator (CFTR) that is responsible for CF was related to defective autophagy and decreased clearance of aggresomes. Further, defective CFTR-induced upregulation of reactive oxygen species (ROS) was associated with crosslinking and functional sequestration of BECN1, one of the key ATG genes responsible for autophagosome formation.

Further, basing on experiments in transgenic animals models directed to ATG genes, it was demonstrated that autophagy deficiency is related to age-related cardiomyopathy and with desmin-related myopathy, in particular, known for accumulation of misfolded proteins and production of soluble pre-amyloid oligomers. It has been suggested that autophagy is an adaptive process that is activated under normal conditions in response to stress to protect the heart from acute and chronic ischemia. This has led to clinical approaches using pharmacological induction of cardiac autophagy to enhance heart's tolerance to ischemia.

Recent studies reveal critical role of autophagy in immunity and inflammation. Autophagy controls inflammation through regulatory interactions with innate immune signaling pathways by removing endogenous inflammasome agonists and through effects on the secretion of immune mediators. Moreover, autophagy contributes to antigen presentation and T cell homeostasis, and it affects T cell repertoires and polarization.

Finally, deficient autophagy was associated with the majority of Lysosomal Storage Disorders (LSDs) that are characterized by progressive accumulation of undigested macromolecules within the cell due to lysosomal dysfunction. The progressive nature of phenotype development is one of the hallmarks of LSDs. Considering the highly integrated function of lysosomes and autophagosomes, contribution of deficient to LSDs is to be expected. Evidence of deficient autophagy were found a number of LSDs, including Glycogenosis Type II (Pompe Disease), Danon disease, Multiple sulfatase deficiency (MSD) and Mucopolysaccharidosis type IIIA (MPS IIIA), Mucopolysaccharidosis type VI (MPS VI), Sphingolipidoses such as Niemann-Pick type C disease (NPC), Gaucher disease, Fabry disease and GM1 gangliosidosis, and in Mucolipidosis types II, III and IV.

In the context of cancer, methods of the present invention are particularly applicable to various types of carcinoma, melanoma, lymphoma, sarcoma, leukemia of various stages, grades and primary origins.

When referring to herein carcinoma is meant any type of cancer of epithelial origin, including the melanoma, which is a particular type of an epithelial skin cancer. Notable examples of carcinoma include, apart from breast, colon and prostate cancer, also basal cell carcinoma, squamous cell carcinoma, renal cell carcinoma, etc.

Under the term lymphoma is meant any one of blood cell tumors developing from lymphocytes, including the two main types—the Hodgkin and non-Hodgkin lymphomas, as well as the two other types, the multiple myeloma and immuno-proliferative diseases, also included by the World Health Organization (WHO) in this category.

Under the term sarcoma is meant is a cancer that arises from transformed cells of mesenchymal origin, including cancellous bone, cartilage, fat, muscle, vascular, or hematopoietic tissues, i.e. bone and soft tissue sarcomas. Human sarcomas are quite rare, as most of the human tumors are of epithelial origin.

Under the term leukemia is meant any one of cancers that begin in the bone marrow and results in high numbers of abnormal white blood cells, including the four main types of leukemia: acute lymphoblastic leukemia (ALL), acute myeloid leukemia (AML), chronic lymphocytic leukemia (CLL) and chronic myeloid leukemia (CML), and less common types.

Further malignancies that may find utility in the present invention can comprise but are not limited to hematological malignancies (including lymphoma, leukemia and myeloproliferative disorders, as described above), hypoplastic and aplastic anemia (both virally induced and idiopathic), myelodysplastic syndromes, all types of paraneoplastic syndromes (both immune mediated and idiopathic) and solid tumors (including GI tract, colon, lung, liver, breast, prostate, pancreas and Kaposi's sarcoma. The invention may be applicable as well for the treatment or inhibition of solid tumors such as tumors in lip and oral cavity, pharynx, larynx, paranasal sinuses, major salivary glands, thyroid gland, esophagus, stomach, small intestine, colon, colorectum, anal canal, liver, gallbladder, extraliepatic bile ducts, ampulla of vater, exocrine pancreas, lung, pleural mesothelioma, bone, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma of the uvea, retinoblastoma, carcinoma of the lacrimal gland, sarcoma of the orbit, brain, spinal cord, vascular system, hemangiosarcoma and Kaposi's sarcoma.

In specific embodiments, methods of the present invention are applicable to carcinomas of the colon, breast and pancreas. Efficacy of methods of the invention in inducing controlled mitochondrial damage, autophagy and necrosis ultimately leading to cell death was presently demonstrated in cancer cells representing breast and colon carcinomas of various stages, including metastatic properties (EXAMPLES 3, 8).

It should be noted that “subject in need” or a “subject” it is meant any organism who may be affected by the above-mentioned conditions, and to whom the methods herein described is desired, including humans, domestic and non-domestic mammals such as canine and feline subjects, bovine, simian, equine and murine subjects, rodents, domestic birds, aquaculture, fish and exotic aquarium fish. It should be appreciated that the subject may be also any reptile or zoo animal. More specifically, the methods of the invention are intended for mammals. In more specific embodiments, when relating in detail to methods of the present invention the step of administering Fer inhibitors or any composition comprising thereof to a subject should be understood as referring to any member of a mammalian species. By “mammalian subject” is meant any mammal for which the proposed therapy is desired, including human, livestock, equine, canine, and feline subjects, most specifically humans. Thus, in certain embodiments of the invention said subject is a human subject.

Further, administration of Fer inhibitors or any composition comprising thereof for therapeutic purposes, i.e. to prevent, overcome, slow down or alleviate a disease or clinical condition in a subject in need thereof, may involve various administration routs. Methods of introduction of therapeutic compounds can be enteral or parenteral and include, but are not limited to, intradermal, intramuscular, intraperitoneal, intravenous, subcutaneous, intranasal, epidural, and oral routes. The compounds may be administered by any convenient route, e.g. by infusion or bolus injection, by absorption through epithelial or mucocutaneous linings (e.g., oral mucosa, rectal and intestinal mucosa) with or without other biologically active agents. Administration can be systemic or local. In addition, it may be desirable to introduce the therapeutic compositions into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In specific embodiments, it may be desirable to administer therapeutic compositions of the invention locally to the area in need of treatment; this may be achieved, for example, and not by way of limitation, by local infusion during surgery, topical application, e.g., by injection, by means of a catheter, or by means of an implant, said implant being of a porous, non-porous, or gelatinous material, including membranes, such as sialastic membranes, fibers, or commercial skin substitutes.

Further, administration regimens may comprise single or multiple doses of bioactive agents and compositions of the invention which are administered daily or weekly, chronically, periodically or by a single administration. Said doses may be single or multiple with equal or gradually increasing or gradually decreasing dose regimens.

In certain embodiments, the effective serum or blood levels of the bioactive ingredient may be achieved within about 10 to about 20 or 30, 40, 50, 60 minutes following its administration. In other embodiments, the effective serum or blood levels of the bioactive ingredient may be achieved within about 5 to about 10, 20 or 30, 40, 50, 60 minutes following its administration.

Still further, methods of the invention may be part of a larger therapeutic regimen, in which they only partially contribute to the improvement of the therapeutic outcomes. In this context, administering of a therapeutically effective amount of bioactive agents and compositions of the invention to a subject may lead to enhancement or improvement of beneficial effects obtained by implementation of conventional and other non-conventional therapies.

Under enhancement or improvement of beneficial effects, and variations thereof, is meant therapeutic effects against placebo, or an increase in the therapeutic effect of a state-of-the-art medical treatment above that normally obtained when a pharmaceutical composition is administered without the bioactive agent of this invention. An increase in the therapeutic effects is manifested when there is an acceleration and/or increase in intensity and/or extent of the therapeutic effects obtained as a result of administering the bioactive agent(s), also including extension of the longevity of therapeutic benefits. It can also manifest where a lower amount of the pharmaceutical composition is required to obtain the same benefits and/or effects when it is co-administered with bioactive agent(s) of the invention as compared to the administration in a higher amount of the pharmaceutical composition in the absence thereof. The enhancing effect preferably, but not necessarily, results in treatment of acute symptoms for which the pharmaceutical composition alone is not effective or is less effective therapeutically. Enhancement is achieved when there is at least a 5% increase in the therapeutic effects, such as at least 10% increase in the therapeutic effects when a bioactive agent of the present invention is co-administered with a pharmaceutical composition compared with administration of the pharmaceutical composition alone. Preferably the increase is at least 25%, more preferably at least 50%, even more preferably at least 75%, most preferably at least 100%.

In this connection, administering or optionally, co-administering or co-administration of bioactive agent(s) of the invention, or bioactive agents and state-of-the-art medicaments, as used herein, refers to the administration of one or more bioactive agents of the present invention, or administration of one or more bioactive agents of the present invention and a state-of-the-art pharmaceutical composition within a certain time period. The time period is preferably less than 72 hours, such as 48 hours, for example less than 24 hours, such as less than 12 hours, for example less than 6 hours, such as less than 3 hours. These terms may also mean that the bioactive agent and a therapeutic composition can be administered together.

Methods and compositions of the invention may be part of a combination therapy that denotes concurrent or consecutive administration of additional therapeutic agents. For example, concurrent administration can mean one dosage form in which the two or more agents are contained whereas consecutive administration can mean separate dosage forms administered to the patient at different times and/or different routes.

Clinical relevance of the inhibition of Fer and its resulting biological outcomes in terms of mitochondrial damage, ATP depletion, modulation of PARP activity and enhancement of autophagy and necrosis were previously discussed.

It is this conceived that in certain embodiments, methods of the present invention are intended to induce ATP depletion in a subject in need thereof, said method comprising the step of administering to said subject an effective amount of at least one inhibitor of Fer. It should be appreciated that in certain embodiments, the method of the invention may induce ATP depletion in at least one cell of the treated subject.

In further embodiments, methods of the present invention are intended to induce necrosis (also necrotic cell death, or programmed necrotic cell death) in a subject, said method comprising the step of administering to said subject an effective amount of at least one inhibitor of Fer. More specifically, the method of the invention may induce necrosis in at least one cell of the treated subject.

In yet other embodiments, methods of the invention are applicable for inducing ADP ribosylation activity of PARP-1 in a subject in need thereof, said method comprising the step of administering to said subject an effective amount of at least one inhibitor of Fer. Still further, it should be understood that the method of the invention may induce ADP ribosylation activity of PARP-1 in at least one cell of the treated subject.

For the purpose of certain clinical applications, methods of the invention may use an inhibitor of Fer that is a compound of the following structure, or a pharmaceutically acceptable salt thereof:

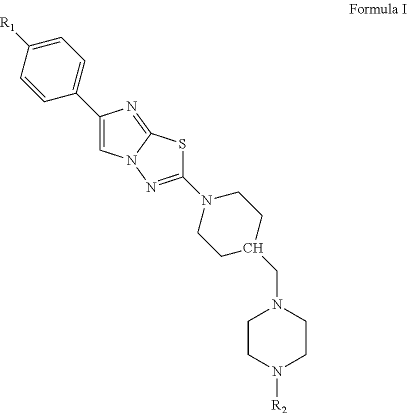

wherein R₁ and R₂ are each independently selected from hydrogen, halogen, C₁₋₆alkyl, C₂₋₆alkenyl, N—(C₁₋₆alkyl)₂ or N—(C₂₋₆alkenyl)₂, and wherein the C₁₋₆alkyl and C₂₋₆alkenyl being straight or branched.

Further, R₁ may be hydrogen, methyl, ethyl, propyl, isopropyl, N-isopropyl, butyl, sec-butyl, tert-butyl, N-butyl, N-sec-butyl, N-tert-butyl, F, Cl, Br or I; and R₂ may be a hydrogen, methyl, ethyl, propyl, isopropyl or butyl.

A specific example of Fer inhibitor is a compound of the above formula wherein R₁ is isopropyl and R₂ is CH3 or any pharmaceutically salts thereof.

Thus in specific embodiments, methods of the invention may use a compound that is 6-(4-isopropyl-phenyl)-2-{4-[(4-methyl-piperazin-1-yl)methyl]piperidin-1-yl}imidazo[2,1-b][1,3,4]thiadiazole, referred to herein as compound “522-0251” or as compound “Z522-0251”, a pharmaceutically acceptable salt thereof, or any composition comprising the same.

In yet further specific embodiments, methods of the invention may use a pharmaceutically acceptable salt of “522-0251”, which is the tartarate salt of 6-(4-isopropyl-phenyl)-2-{4-[(4-methyl-piperazin-1-yl)methyl]piperidin-1-yl}imidazo[2,1-b][1,3,4]thiadiazole, designated E260 and also referred to herein as 0260. Tartarate refers to the tartrate dianion is O⁻OC—CH(OH)—CH(OH)—COO⁻ or C₄H₄O₆²⁻.

Still further, in specific embodiments, methods of the invention may use a micellar formulation of E260 (also referred to herein as 0260), which was presently demonstrated as particularly advantageous in achieving the desired effects in cancer cells. In more specific embodiments said micellar formulation of the invention may be referred to as o/w micellar formulation of E260, as defined herein before.

Thus, methods according to the above may be particularly applicable to treat a subject suffering from autophagic diseases or conditions.

More specifically, methods of the present invention are applicable for the treatment, amelioration, inhibition or prevention of an autophagic diseases or condition in a subject in need thereof, said method comprising the step of administering to said subject a therapeutically effective amount of at least one inhibitor of Fer.

The terms treating, treatment or therapy as used herein refer equally to curative therapy, prophylactic or preventative therapy and ameliorating therapy. The term includes an approach for obtaining beneficial or desired therapeutic effect, which may be established clinically by means of physiological, metabolic or biochemical parameters. In this application of invention, beneficial or desired clinical results include, but are not limited to, alleviation of symptoms, diminishment of extent of disease, stabilized (i.e., not worsening) condition, delay or slowing of progression or worsening of condition/symptoms, amelioration or palliation of the condition or symptoms, and remission (whether partial or total), whether detectable or undetectable. The term palliation and variations thereof, as used herein, means that the extent and/or undesirable manifestations of a physiological condition or symptom are lessened and/or time course of the progression is slowed or lengthened, as compared to not administering compositions of the present invention.

A treatment effect or therapeutic effect is manifested if there is a change in the condition being treated, as measured by the clinical criteria acknowledged as conclusive with regard to the treatment outcomes. There is a change in the condition being treated if there is at least 5% improvement according to said criteria, preferably 10% improvement, more preferably at least 25%, even more preferably at least 50%, such as at least 75%, and most preferably at least 100% improvement. For example, the change can be based on improvements in the severity of symptoms in an individual patient, or on a difference in the frequency of improved conditions in populations of individuals with and without treatment with bioactive agents of the present invention or compositions thereof, specifically the micellar E260 formulations of the invention.

Further in this connection, bioactive agents or compositions the invention may induce reduction, prevention, inhibition, or reversal of pathological symptoms of the relevant clinical conditions. The terms reduction, inhibition or reversal of pathological symptoms, as used herein, refer to complete or partial reduction or reversal of pathological symptoms of a disorder to which the present invention is particularly applicable. Pathological symptoms are usually categorized or graded according to specific clinical criteria and/or scales that have been evaluated and approved for medical use and are considered conclusive as regards primary diagnosis and severity of the disorder. Thus, reduction or inhibition of pathological symptoms refers to a reduction with respect to appropriate clinical scales or reduction in the number of relevant clinical indices.

A therapeutically effective amount (also pharmacologically or pharmaceutically or physiologically effective amount which are used herein equally) is the amount of an bioactive agent present in a pharmaceutical composition that is needed to provide an desired level of active agent in the bloodstream or at the target organ of an organism to be treated to give an anticipated physiological response when such composition is administered. The precise amount will depend upon numerous factors, e.g., the active agent, the activity of the composition, the delivery device employed, the physical characteristics of the composition, intended patient use (i.e., the number of doses administered per day), patient considerations, and the like, and can readily be determined by one skilled in the art, based upon the information provided herein. An effective amount of a bioactive agent of the invention, specifically the micellar E260 formulations of the invention, can be administered in one administration, or through multiple administrations of an amount that total an effective amount, preferably within a 24-hour period. It can be determined using standard clinical procedures for determining appropriate amounts and timing of administration. It is understood that the effective amount can be the result of empirical and/or individualized (case-by-case) determination on the part of the treating health care professional and/or individual.

Therapeutically effective amount of the bioactive agent of the invention, specifically the micellar E260 formulations of the invention, refers to an amount of from about 0.01 ng to about 1000 mg bioactive agent, specifically the micellar E260 formulations of the invention, per kg body weight (bw) of the subject treated by this method per day. Further, this range can be from each of 0.01, 0.1, 1, 10, 20, 30, 40, 50, 60, 70, 80, 90, 100, 200, 300, 400, 500, 600, 700, 800, 900, or 1000 ng that are equivalent to about 1 microgram. In more specific embodiments this range can be from each of 0.01, 0.1, 1, 10, 20, 30, 40, 50, 60, 70, 80, 90, 100, 200, 300, 400, 500, 600, 700, 800, 900, or 1000 microgram or μg that are equivalent to about 1 milligram. In more specific embodiments this range can be from each of 0.01, 0.1, 1, 10, 20, 30, 40, 50, 60, 70, 80, 90, 100, 200, 300, 400, 500, 600, 700, 800, 900, or 1000 milligrams or mg, and even more. It should be appreciated that the effective dose may change and may be adapted according to the administration route (for example, i.p. or i.v.) and severity of the disorder.

In yet some further embodiments, the effective amount of the bioactive agent of the invention, specifically the micellar E260 formulations of the invention may be any amount effective to treat a subject suffering from any autophagic disease or condition, or any amount suitable for achieving induction of at least one of necrosis, mitochondrial damage and autophagy in at least one cell of a subject in need thereof. Therefore, the Fer inhibitor of the invention may be presented in micromolar or nanomolar amounts, specifically, such amount may range between about 100 μM to about 0.01 nM. More specifically, an effective amount of the micellar formulation of 0260 may be any one of 100 μM, 90 μM, 80 μM, 70 μM, 60 μM, 50 μM, 40 μM, 30 μM, 20 μM, 10 μM, 9.5 μM, 9 μM, 8.5 μM, 8 μM, 7.5 μM, 7 μM, 6.5 μM, 6 μM, 5.5 μM, 5 μM, 4.5 μM, 4 μM, 3.5 μM, 3 μM, 2.5 μM, 2 μM, 1.5 μM, 1 μM, 0.5 μM or less, specifically, 450 nM, 400 nM, 350 nM, 300 nM, 250 nM, 200 nM, 150 nM, 100 nM or less, for example, 90, 80, 70, 60, 50, 40, 30, 20, 10, 9, 8, 7, 6, 5, 4, 3, 2, 1, 0.5, 0.4, 0.3, 0.2, 0.1 nM or less, specifically, 0.05, 0.04, 0.03, 0.02, 0.01 or even less.

In yet some further specific embodiments, the effective amount of the micellar formulation of the invention may range between about 100 nM to about 10 μM, specifically, 100, 200, 300, 400, 500, 600, 700, 1000, 1100, 1200, 1300, 1400, 1500, 1600, 1700, 1800, 1900, 2000, 2100, 2200, 2300, 2400, 2500, 2600, 2700, 2800, 2900, 3000 nM or more, specifically, 4000, 4500, 5000, 5500, 6000, 6500, 7000, 7500, 800, 8500, 9000, 9500, or 10000 nM that are equivalent to 10μM. In yet some further specific embodiments, the effective amount may range between about 1, 2, 3, 4, 5, 6, 7, 8, 9, 10 μM or more, specifically, 2 μM.

It should be further appreciated that the micellar formulation of the invention demonstrates superiority over non-micellar formulations of the 0260 compound. For example, the effective amount of the non-micellar formulations of 0260 may range between about 20 μM to 100 μM. Moreover, the 0260 non-micellar formulation is comprised of a maximum 1 mM 0260 dissolved in 100% DMSO. This formulation is inferior to the micellar formulation by the effectivity of the compound solubility, stability and cell penetration.

Clinical conditions for which methods of the present invention are applicable were previously discussed in detail, including various types of cancer, neurodegenerative disorders and various disorders associated with accumulation of misfolded and/or ubiquitinated protein aggregates with a perturbation of autophagy.

Among clinical conditions which could particularly benefit from application of treatment methods of the present invention are various types of cancer and metastatic cancers in particular.

Thus, in specific embodiments therapeutic methods of the present invention are applicable for treating a metastatic cancer disease of types: metastatic carcinoma, melanoma, sarcoma, lymphoma or leukemia.

In yet other embodiments, therapeutic methods of the invention are applicable for treating colon carcinoma, breast carcinoma and pancreatic carcinoma.

More specifically, colon cancer (also referred to herein as “colorectal cancer or carcinoma”) as herein defined is a disease in which malignant (cancer) cells form in the tissues of the colon (part of the body's digestive system). Colorectal carcinoma is the third most common cancer in the United States after prostate and lung/bronchus cancers in men and after breast and lung/bronchus cancers in women. Risk factors of colon cancer include age and health history, a family history of colon cancer, and in some cases having inflammatory bowel disease (IBD). The results of different studies vary, but in general, the risk of colon cancer for people with IBD increases by 0.5% to 1% yearly approximately 8 to 10 years after diagnosis.

The prognosis of patients with colon cancer is clearly related to the degree of penetration of the tumor through the bowel wall, the presence or absence of nodal involvement, and the presence or absence of distant metastases, with these three characteristics forming the basis for all staging systems developed for this disease.

Many other prognostic markers have been evaluated retrospectively for patients with colon cancer, though most, including allelic loss of chromosome 18q or thymidylate synthase expression, have not been prospectively validated.

It should be noted that the invention provides method and compositions that are specifically applicable for metastatic types of colon cancer.

In further particular embodiments, the methods and compositions of the invention are applicable for the treatment of pancreatic cancer or carcinoma. The term “pancreatic cancer” is meant herein refers to a malignant neoplasm originating from transformed cells arising in tissues forming the pancreas, including adenocarcinomas arising within the exocrine component of the pancreas (accounting for 95% of pancreatic cancer) and also rarer cancers that arise from islet cells—classified as neuroendocrine tumors. Pancreatic cancers exhibit an extremely poor prognosis (the 1- and 5-year relative survival rates are 25% and 6%, respectively, for all stages combined; median survival for locally advanced and for metastatic disease, which collectively represent over 80% of individuals, is about 10 and 6 months respectively). Gender and ethnicity are known risk factors for developing pancreatic cancer, men are 30% more likely to get pancreatic cancer than are women; and individuals of African descent are more likely to develop pancreatic cancer than white individuals.

Still further embodiments of the invention are particularly applicable for treating breast cancer. When presently referring to breast cancer, is meant any type of cancer originating from breast tissue, including ductal and lobular carcinomas. The present context also encompasses genetic or hereditary breast cancers (5-10% of all cases) developing from predisposing mutations in BRCA1 and BRCA2 genes and also other relevant mutations in p53 (Li-Fraumeni syndrome), PTEN (Cowden syndrome), and STK11 (Peutz-Jeghers syndrome), CHEK2, ATM, BRIP1, and PALB2 genes. The present context also encompasses all breast cancer classifications, including those using histopathology (e.g. mammary ductal carcinoma, carcinoma in situ, invasive carcinoma or inflammatory breast cancer), grade (e.g. well differentiated/low grade, moderately differentiated/intermediate grade and poorly differentiated/high grade), stage (0=pre-cancerous, 1-3=regional, 4=metastatic), receptor status (relating to the expression of estrogen receptor ER, PR progesterone receptor and/or HER2/ERBB2 receptor), DNA and protein based classification (using specific mutations or gene expression profiles), and other classification approaches. In more specific embodiments, the methods and compositions of the invention are applicable for treating breast cancer of grade 4.

More recently, DNA multi-gene expression profiles have been used to categorize breast cancers into molecular subtypes that generally correspond to immunohistochemistry receptor status, one commercial source is the BluePrint test. In the present classification, breast cancer cells may be classified as ER positive (ER+), ER negative (ER−), PR positive (PR+), PR negative (PR−), HER2 positive (HER2+), and HER2 negative (HER2−). Cells with none of these receptors are called triple negative.

ER+ cancers cells can be treated with drugs to reduce either the effect of estrogen (e.g. tamoxifen) or the actual level of estrogen (e.g. aromatase inhibitors), and generally have a better prognosis. Prior to modern treatments, HER2+ breast cancer had the worse prognosis. Today however treatment with trastuzumab in combination with conventional chemotherapy has significantly improved the prognosis of this type of cancer. Conversely, triple negative cancer (i.e. no positive receptors), lacking targeted treatments now has a comparatively poor prognosis.

In yet another embodiment, the present invention provides a method for treating tripe-negative breast cancer. As used herein the term “triple negative” or TN, refers to cancers that do not express the ERBB2, the ER and the Progesteron receptor, beyond the clinical threshold. This specific subtype tends to exhibit more aggressive characteristics and a worse prognosis. Further, “triple negative” endometrial (or uterine) cancers were also reported, in which “triple negative” phenotype was is associated with advanced stage, high grade, and high risk histology, as well as poor survival.

Further, due to the reasons discussed above, methods of the present invention may be part of birth control regiments, more specifically serving as male contraceptives.

Two main forms of medication-based male contraceptive are currently under study: the male hormonal contraceptives that can be taken in pill form, similar to the existing oral contraceptive pill for women, and male hormonal injections. In addition there are methods like Reversible Inhibition of Sperm Under Guidance (RISUG or RISUG/VasalGel, which has completed a small phase II clinical trial in humans in India) and ultrasound (with results so far obtained in experimental animals). Many of these medications, however, are suffering from not sufficient efficacy and/or significant adverse effects. Major medication-based approaches that are currently under trial include: Gossypol an extract of cotton that decreases sperm production, but is permanent in only in 20% of people; inhibition of chromatin remodeling by selective inhibitors of Bromodomain Testis-Specific Protein (BRDT) may produce reversible sterility in male mice; immunocontraception targeting sperm antigens that was found effective in male primates; calcium channel blockers, such as Nifedipine, which cause reversible infertility by altering the lipid metabolism of sperm; compounds that interfere with the vitamin A pathway that may render male mice sterile without affecting libido; Adjudin and Gamendazole, lonidaminea analogs that may cause reversible infertility; and multiple male hormonal contraceptive protocols.

Thus, methods of the present invention may be applicable per se as male contraceptives or in combination with any one of the above mentioned approaches.

Thus for applications pertaining to male contraception, methods of the present invention may be applicable for inducing any one mitochondrial damage, autophagy and cell death in sperm cells of a subject, said method comprising the step of administering to said subject or to at least one further subject being in contact with said subject or with said sperm cell/s, a spermicidal effective amount of at least one inhibitor of Fer. In this case a subject is a male subject.

Further applications pertaining to birth control may include methods of the present invention serving as sperimicides. Unlike male contraceptives, spermicide is a contraceptive substance that destroys sperm, inserted vaginally prior to intercourse to prevent pregnancy.

Thus in these applications, methods of the present invention are applicable for inducing any one mitochondrial damage, autophagy and cell death in sperm cells of a subject, said method comprising the step of administering to said subject or to at least one subject being in contact with said subject or with said sperm cell/s, a spermicidal effective amount of at least one inhibitor of Fer. In this case a subject is a female subject.

In other clinical applications, as discussed above, methods of the present invention may be applicable for inducing ADP ribosylation activity of PARP in a subject in need thereof, the method comprising the step of administering to said subject an effective amount of at least one inhibitor of Fer, thereby enhancing the activity of PARP in said cell.

Still further, in another aspect, the invention provides a method for treating, preventing, inhibiting, reducing, eliminating, protecting or delaying the onset of an autophagic disease or condition in a subject. More specifically, the method comprises the step of administering to the subject a therapeuticall effective amount of at least one inhibitor of Fer or any vehicle, matrix, nano- or micro-particle, or composition comprising the same. In more specific embodiments, the subject is suffering from an autophagic diseases or condition. In more specific embodiments, autophagic diseases or condition may be any one of cancer, protein conformational disorder (PCD), a neurodegenerative disease or condition, inflammatory disorder, metabolic disorder or a myopathy.

Yet it is another specific aspect of the present invention to provide a micellar formulation comprising a compound or a pharmaceutically acceptable salt thereof of the following structure of formula I:

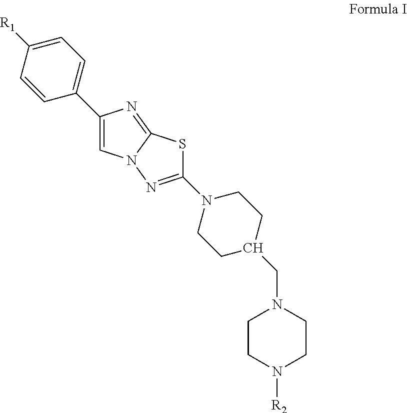

wherein R₁ and R₂ are each independently selected from hydrogen, halogen, C₁₋₆alkyl, C₂₋₆alkenyl, N—(C₁₋₆alkyl)₂ or N—(C₂₋₆alkenyl)₂, and wherein the C₁₋₆alkyl and C₂₋₆alkenyl being straight or branched; said compound or a pharmaceutically acceptable salt thereof being incorporated in micelles, the micelles are formed by mixing a nonionic surfactant such as alcohol alkoxylates (e.g., polyethoxylated castor oil such as Cremophor® EL), C₂₋₆alcohol (e.g., ethanol) and a buffer selected from acetate, phosphate and sulfate (e.g., PBS), wherein the diameter of said micelle ranges between about 1 nm to about 100 nm. It should be appreciated that in certain embodiments, the micellar formulations of E260, according to the invention may be o/w micellar formulations as defined herein before.

Further, in the micellar formulation according to the invention R₁ may be selected from hydrogen, methyl, ethyl, propyl, isopropyl, N-isopropyl, butyl, sec-butyl, tert-butyl, N-butyl, N-sec-butyl, N-tert.-butyl, F, Cl, Br, or I; and R₂ may be selected from hydrogen, methyl, ethyl, propyl, isopropyl or butyl.

In specific embodiments, the micellar formulation according to the invention comprises a compound which is 6-(4-isopropyl-phenyl)-2-{4-[(4-methyl-piperazin-l-yl)methyl]piperidin-1-yl}imidazo[2,1-b][1,3,4]thiadiazole, or a pharmaceutically acceptable salt thereof.

In further embodiments, the pharmaceutically acceptable salt is tartarate.

Further, the micellar formulation according to the invention may include surfactant which is at least one of Cremophor® EL, Triton X-100, Triton X-114, NP-40, Brij-35, Brij-58, Tween 20, Tween 80, octyl glucoside and octyl thioglucoside, said solvent is at least one of ethanol, propanol, isopropanol or butanol and said buffer is a phosphate buffer or others.

For the purpose of specific embodiments, said surfactant is Cremophor® EL, said solvent is ethanol or propanol and said buffer is a phosphate saline buffer.

According to certain embodiments, the proportions (or ratios) between the constitutes of the micellar formulation of the invention is between about 1 to 25%, Cremophor® EL, between about 1 to 25% ethanol and between about 50 to 98% PBS. More specifically, about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24% and 25% or more Cremophor® EL, about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24% and 25% ethanol and about 50%, 51%, 52%, 53%, 54%, 55%, 56%, 57%, 58%, 59%, 60%, 61%, 62%, 63%, 64%, 65%, 66%, 67%, 68%, 69%, 70%, 271%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97% and 98% PBS.

For the purpose of specific embodiments, the micelles of the invention may be formed by a combination of catastrophic phase inversion with phase inversion temperature.

It yet another aspect of the present invention to provide a composition comprising a micellar formulation of a compound or a pharmaceutically acceptable salt thereof, said compound is of the following structure of formula I:

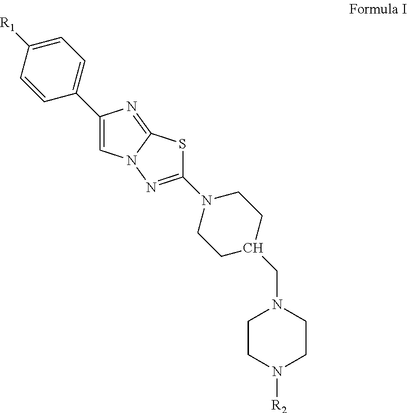

wherein R₁ and R₂ are each independently selected from hydrogen, halogen, C₁₋₆alkyl, C₂₋₆alkenyl, N—(C₁₋₆alkyl)₂ or N—(C₂₋₆alkenyl)₂, and wherein the C₁₋₆alkyl and C₂₋₆alkenyl being straight or branched; incorporated in micelles, the micelles are formed by mixing a nonionic surfactant such as alcohol alkoxylates, C₂₋₆alcohol and a buffer selected from acetate, phosphate and sulfate, wherein the diameter of said micelle ranges between about 1 nm to about 100 nm, said composition optionally further comprises at least one of pharmaceutically acceptable carrier/s, diluent/s and/or excipient/s. In yet some further specific embodiments the diameter of the micelle of the invention may be any one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 85, 87, 88, 89, 90, 91, 92, 93, 94, 95, 96, 97, 98, 99 or about 100 nm or more. Still further, specific embodiments relate to the micellar formulations of the invention having a diameter of between about 5 nm to about 30 nm, specifically, 15, 16, 17, 18, 19, 20 nm or more.

Further, in this composition R₁ may be represented by hydrogen, methyl, ethyl, propyl, isopropyl, N-isopropyl, butyl, sec-butyl, tert-butyl, N-butyl, N-sec-butyl, N-tert-butyl, F, Cl, Br or I; and R₂ may be presented by hydrogen, methyl, ethyl, propyl, isopropyl or butyl.

In specific embodiments, the composition of the invention comprises the compound 6-(4-isopropyl-phenyl)-2-{4-[(4-methyl-piperazin-1-yl)methyl]piperidin-1-yl}imidazo[2,1-b][1,3,4]thiadiazole.

In specific embodiments, the composition of the invention comprises an effective mount of compound designated E260, being a tartarate salt of 6-(4-isopropyl-phenyl)-2-{4-[(4-methyl-piperazin-1-yl)methyl]piperidin-1-yl}imidazo[2,1b][1,3,4]thiadiazole.

Other pharmaceutically acceptable salts include those formed with free amino groups such as those derived from hydrochloric, phosphoric, acetic, oxalic, acids, etc., and those formed with free carboxyl groups such as those derived from sodium, potassium, ammonium, calcium, ferric hydroxides, isopropylamine, triethylamine, 2-ethylamino ethanol, histidine, procaine, etc.

It is another purpose of the present invention to provide a pharmaceutical composition comprising said micellar formulation in an amount effective for inducing in a cell at least one of, mitochondrial damage and ADP ribosylation activity of poly(ADP-ribose) polymerase 1 (PARP), and further optionally comprising a pharmaceutically acceptable carrier.

In this connection, an effective amount is also a therapeutically effective amount. The term pharmaceutically acceptable means approved by a regulatory agency of the US FDA or listed in the U.S. Pharmacopeia for use in animals and more particularly in humans.

The term carrier refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin.

In specific embodiments, the composition of the invention may be formulated in accordance with routine procedures as a pharmaceutical composition adapted for intravenous or intraperitoneal administration to human beings. Where necessary, the composition may also include a solubilizing agent and a local anesthetic such as lidocaine to ease pain at the site of the injection. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The amount of the active agent of the invention which will be effective in the treatment of a relevant condition can be determined by standard clinical techniques based on the present description. In addition, in vitro assays may optionally be employed to help identify optimal dosage ranges. The precise dose to be employed in the formulation will also depend on the route of administration, and the seriousness of the condition, and should be decided according to the judgment of the practitioner and each subject's circumstances. However, suitable dosage ranges for intravenous or intraperitoneal administration are generally about 0.1 ng to 1000 milligrams of active compound per kilogram body weight. Effective doses may be extrapolated from dose-response curves derived from in vitro or animal model test systems.

In certain embodiments, the invention may further use drug delivery methods. Drug delivery refers to approaches, formulations, technologies and systems for transporting a pharmaceutical compound in the body as needed to safely achieve its desired therapeutic effect. It may involve scientific site-targeting within the body, or it might involve facilitating systemic pharmacokinetics; in any case, it is typically concerned with both quantity and duration of drug presence. Drug delivery is often approached via a drug's chemical formulation, but it may also involve medical devices or drug-device combination products. Drug delivery is a concept heavily integrated with dosage form and route of administration, the latter sometimes even being considered part of the definition.

Drug delivery technologies modify drug release profile, absorption, distribution and elimination for the benefit of improving product efficacy and safety, as well as patient convenience and compliance. Drug release is from: diffusion, degradation, swelling, and affinity-based mechanisms. Most common routes of administration include the preferred non-invasive peroral (through the mouth), topical (skin), transmucosal (nasal, buccal/sublingual, vaginal, ocular and rectal) and inhalation routes. Many medications such as small molecules, peptide and protein, antibody, vaccine and gene based drugs, in general may not be delivered using these routes because they might be susceptible to enzymatic degradation or cannot be absorbed into the systemic circulation efficiently due to molecular size and charge issues to be therapeutically effective.

Current efforts in the area of drug delivery include the development of targeted delivery in which the drug is only active in the target area of the body (for example in cancerous tissues) and sustained release formulations in which the drug is released over a period of time in a controlled manner from a formulation. In order to achieve efficient targeted delivery, the designed system must avoid the host's defense mechanisms and circulate to its intended site of action. Types of sustained release formulations include liposomes, drug loaded biodegradable microspheres and drug polymer conjugates.

Further, for the purpose of specific embodiments, the pharmaceutical composition of the invention according to the above is targeted to induce mitochondrial damage which may comprise at least one of mitochondrial membrane potential (MMP) depolarization, deformation of mitochondrial structure/s and down regulation of mitochondrial protein/s.

In other embodiments, this mitochondrial damage may result in down regulation of mitochondrial proteins, for example, Hexokinase II.

Still further embodiment concern a composition of the invention for inducing autophagy in said cell, said method comprising the step of contacting said cell with an effective amount of at least one inhibitor of Fer.

It should be noted that the invention further encompasses a composition for modulating autophagy in a cell, by contacting the cell with an effective amount of at least one of Fer polypeptide or any functional fragments thereof, and at least one inhibitor of Fer, thereby modulating, specifically increasing or decreasing autophagy in said cell.

Thus, in an alternative embodiment, the invention provides a composition for reducing autophagy in a cell, by contacting the cell with an effective amount of Fer polypeptide or a functional fragment thereof.

In certain embodiments, compositions of the invention may be used for inducing ATP depletion in a cell, by contacting said cell with an effective amount of at least one inhibitor of Fer.

Further embodiments relate to compositions of the invention for inducing necrosis (or necrotic cell death, or programmed necrotic cell death) in a cell, by contacting said cell with an effective amount of at least one inhibitor of Fer, specifically, the micellar E260 formulation of the invention.

In further embodiments, compositions of the invention may be applicable for enhancing ADP ribosylation activity of PARP 1 in a cell, by contacting said cell with an effective amount of at least one inhibitor of Fer.

In certain embodiments, compositions of the invention may be applicable for enhancing ADP ribosylation activity of PARP 1 in a cell, by contacting the cell with an effective amount of at least one inhibitor of Fer.

Alternatively, the invention provides compositions for modulating, specifically, reducing ADP ribosylation activity of PARP 1 in a cell, by contacting the cell with an effective amount of Fer polypeptide or a functional fragment thereof.

It should be noted that in certain embodiments, the cell may be of a subject suffering from an autophagic diseases or condition. More specifically, such autophagic diseases or condition may be any one of cancer, protein conformational disorder (PCD), a neurodegenerative disease or condition (e.g. Alzheimer's disease, Parkinson's disease, Huntington's disease, Creutzfeldt-Jakob's disease), inflammatory disorder, metabolic disorder or a myopathy (e.g. cardiomyopathy).

The invention therefore provides a composition for inducing autophagy in a subject in need thereof.

In yet some other embodiments, the cell may be any one of a cancerous cell or a sperm cell.

In some specific embodiments, the cancerous cell may be a metastatic cancer cell.

In still further embodiments, the invention further provides a composition for inducing ADP ribosylation activity of PARP-1 in a subject in need thereof.

In yet another aspect, a pharmaceutical composition of the invention comprises a micellar formulation of the compound 6-(4-isopropyl-phenyl)-2-{4-[(4-methyl-piperazin-1-yl)methyl]piperidin-1-yl}imidazo[2,1-b][1,3,4]thiadiazole or any salts thereof, specifically tartrate salts thereof, as specifically referred to herein as, E260 or 0260 in an amount effective for inducing in a subject at least one of, specific mitochondrial damage and ADP ribosylation activity of PARP. It should be appreciated that any other Fer inhibitor may be used.

In certain embodiments, this pharmaceutical composition comprises the micellar formulation of the compound 6-(4-isopropyl-phenyl)-2-{4-[(4-methyl-piperazin-1-yl)methyl]piperidin-1-yl}imidazo[2,1-b][1,3,4]thiadiazole, or any tartarate salt thereof in an amount effective for use in a method of treatment, amelioration, inhibition or prevention of an autophagic diseases or condition in a subject in need thereof. The method comprising the step of administering to said subject a therapeutically effective amount of said compound. It should be appreciated that any other Fer inhibitor may be used.

In other embodiments, this pharmaceutical composition of the invention may comprise the micellar formulation the compound 6-(4-isopropyl-phenyl)-2-{4-[(4-methyl-piperazin-1-yl)methyl]piperidin-1-yl}imidazo[2,1-b][1,3,4]thiadiazole in an amount effective for use in a method of treatment, amelioration, inhibition or prevention of a cancer disease or condition in a subject in need thereof, said method comprising the step of administering to said subject a therapeutically effective amount of said compound. It should be appreciated that any other Fer inhibitor may be used.

In further embodiments, said pharmaceutical composition may be applicable for use in a method for inducing in a cell at least one of, mitochondrial damage and ADP ribosylation activity of poly(ADP-ribose) polymerase 1 (PARP).

In still further embodiments, the pharmaceutical composition of the invention may be applicable for use in a method for inducing in a subject at least one of, specific mitochondrial damage and ADP ribosylation activity of poly(ADP-ribose) polymerase 1 (PARP).

It is another aspect of the present invention to provide a spermicidal composition comprising an effective amount of at least one inhibitor of Fer tyrosine kinase (Fer). In yet some specific embodiments, the spermicidal composition of the invention may comprise any of the Fer inhibitors described by the invention in an amount effective for inducing mitochondrial damage in sperm cell/s of a subject.

In some specific embodiments, the spermicidal composition of the invention may comprise a micellar formulation the compound 6-(4-isopropyl-phenyl)-2-{4-[(4-methyl-piperazin-1-yl)methyl]piperidin-1-yl}imidazo[2,1-b][1,3,4]thiadiazole, or any tartarate salt thereof in an amount effective for inducing mitochondrial damage in sperm cell/s of a subject. It should be appreciated that any other Fer inhibitor may be used.

In further embodiments, the spermicidal composition according to the above is applicable for use in a method for inducing mitochondrial damage in sperm cells of a subject.

In yet some further specific embodiments, the spermicidal composition of the invention may be particularly used in a method of reducing or inhibiting at least one of sperm motility and sperm viability.

In further specific embodiments, the spermicidal composition of the invention may be used in a method for reducing or inhibiting progressive sperm motility.

Still further, the invention provides in another aspect thereof methods for reducing or inhibiting at least one of sperm motility and sperm viability. More specifically, the method of the invention may comprise the step of contacting at least one sperm cell of a subject with a spermatocidal effective amount of at least one inhibitor of Fer, specifically, the micellar formulation of the invention. In more specific embodiments, the invention provides methods for reducing or inhibiting sperm motility, specifically, progressive and or total sperm motility in a subject. The method may comprise the steps of administering to said subject or to at least one other subject being in contact with said subject and/or with said sperm cells, an effective amount of the 0260 micellar formulation of the invention.

The present invention features spermicidal compositions and methods of inactivating spermatozoa. Spermicide according to the present invention is a contraceptive substance that affects at least one of the viability and the motility of sperm cells.

More specifically, sperm is the male reproductive cell, being the smaller gamete. A uniflagellar sperm cell that is motile is referred to as a spermatozoon or spermatozoa, which serve to fertilize the ovum, whereas a non-motile sperm cell is referred to as a spermatium. Sperm cells are haploid and cannot divide having a limited life span. In mammals sperm develops in the testicles and is released from the penis.

The mammalian sperm cell consists of a head, a midpiece and a tail. The head contains the nucleus with densely coiled chromatin fibers, surrounded anteriorly by an acrosome, which contains enzymes used for penetrating the female egg. The midpiece has a central filamentous core with many mitochondria spiralled around it, used for ATP production for the journey through the female cervix, uterus and uterine tubes. The tail or “flagellum” executes the lashing movements that propel the spermatocyte.

Motile sperm cells typically move via flagella and require a water medium in order to swim toward the egg for fertilization. In animals most of the energy for sperm motility is derived from the metabolism of fructose carried in the seminal fluid. This takes place in the mitochondria located in the sperm's midpiece (at the base of the sperm head).

Sperm motility describes the ability of sperm to move properly through the female reproductive tract (internal fertilization) or through liquid (external fertilization) to reach the egg. Sperm motility can also be thought of as the quality, which is a factor in successful conception. Specifically, sperm that do not “swim” properly will not reach the egg in order to fertilize it. Sperm motility in mammals also facilitates the passage of the sperm through the cumulus oophorus (a layer of cells) and the zona pellucid (a layer of extracellular matrix), which surround the mammalian oocyte.

More specifically, spermatozoa, after passage through the epididymis, are motile cells. Sperm motility becomes critical at the time of fertilization because it allows or at least facilitates passage of the sperm through the zona pellucida. Hence, assessing the fraction of a sperm population that is motile is perhaps the most widely-used measure of semen quality.

In evaluating motility with most species, sperm are classified as non-motile, progressively motile or non-progressively motile. A progressively motile sperm swims forward in an essentially straight line, whereas a non-progressively motile sperm swims, but with an abnormal path, such as in tight circles.

Another term that is sometimes used is “total motility” which refers to the fraction of sperm that display any type of movement. This concept is rarely used for evaluating animal semen, but is the norm for describing human sperm motility.

There is considerable variability among species in what is considered normal or acceptable motility. For example, to pass a breeding soundness examination, it is recommended that bulls have greater than 30% progressively motile sperm, stallions greater than 60% and dogs greater than 70%. Normal humans typically have total sperm motility of greater than 50%.

Sperm movement is activated by changes in intracellular ion concentration, increase in pH and cAMP.

The tail of the sperm, the flagellum confers motility upon the sperm, and has three principal components: (a) a central skeleton constructed of 11 micotubulescollectively termed the axoneme; (b) a thin cell membrane covering the axoneme; and (c) mitochondria arranged spirally around it the axoneme.

Back and forth movement of the tail results from a rhythmical longitudinal sliding motion between the anterior and posterior tubules making up the axoneme. The energy for this process is supplied by ATP produced by mitochondria. The velocity of a sperm in fluid medium is usually 1-4 mm/min. This allows the sperm to move towards an ovum in order to fertilize it.

In certain embodiments, the spermicidal compositions and methods of the invention may reduce, eliminate, decrease, attenuate or inhibit at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%, of sperm motility, specifically, progressive motility.

It should be appreciated that the spermicidal active ingredients, specifically the Fer inhibitors of the invention, and contraceptive compositions containing the same, may be delivered to the vagina of a female mammal by any means known to those skilled in the art. Typical forms for delivery of the compositions include, for example; creams, lotions, gels, foams, intervaginal devices such as sponges, condoms, including female condoms, suppositories, and films. In addition, the spermicidal compositions of the invention may be used as personal careproducts, such as, for example, condom lubricants, and the like. Such lubricants may comprise commonly known ingredients such as, for example: humectants; e. g., glycerin, sorbitol, mannitol, glycols and glycol ethers; buffers, e. g., glucono-d-lactone; germicidesor bactericides; e. g., chlorhexidine gluconate; preservatives, e. g., methylparaben; viscosifiers; e. g., hydroxyethyl cellulose, etc.; other adjuvants; e. g., colors and fragrances; in addition to the compositions of the present invention. Those skilled in the art will recognize that the physical properties, e. g., viscosity, of such delivery forms may vary widely. For example, the viscosity of a gel form of the composition of the present invention may be substantially higher than the viscosity of lotion form of the composition of the present invention. Further details concerning the materials, ingredients, proportions and procedures of such delivery forms can be selected in accordance with techniques well-known in the art.

The spermicidal compositions of the present invention may be administered to a site for contacting sperm, such as to the vagina of a female mammal, or alternatively to the sperm duct of a male mammal, in a dosage which is effective to immobilize sperm, e. g. sperm present in the vagina, and/or to inhibit sperm motility, specifically progressive motility and thereby the sperm penetration of cervical mucus.

Intervaginal devices also may be used to aid in the administration of the spermicidal active ingredients or contraceptive compositions containing the same. In administering the spermicidal active ingredients in the form of the above compositions, the compositions also may be formulated to release the spermicide both rapidly and/or with a prolonged release of the drug.

It should be further appreciated that the composition of the present invention may be useful as a spermicide and in some embodiments, as antiseptic, alone or in combination with a condom or a diaphragm. By affecting spermatozoa, the spermicidal compositions of the invention may be also useful to protect against the transmission of various sexually transmitted diseases such as viral (HIV or AIDs, herpes, hepatitis B cytomegalovirus), chlamydia, trichomonas, various bacteria including gonorrhea and G. Vaginalis and test strain of Treponema phagedenis, a surrogate for syphilis.

In yet some further embodiments, the composition of the present invention may be specially formulated for vaginal self-application by females. The female adult vagina is normally about five inches in length, and it is important that, for maximum effectiveness, the composition be disposed all the way along the vaginal wall and particularly at the inner end thereof, adjacent to the cervix. Accordingly, a specific embodiment of the present invention provides the composition as described above in combination with an applicator which the patient can use to ensure proper application of composition to the most beneficial location. The applicator is a vaginally insertable elongated object about five inches in length, adapted to receive and dispense the formulation.

In some embodiments, the applicator may be a once usable tube containing a single dosage of formulation, and has an aperture, specifically, equipped with a rupturable removable cap, provided at the distal end thereof, i. e., the end to be disposed adjacent to the patient's cervix on full and proper insertion into the vagina. The internal volume of the tubular applicator allows discharge of the appropriate dosage of composition to be administered. Normally about 5 ml of formulation should be discharged. The remainder remains in, and is discarded with, the applicator. Another form of applicator is equipped with a plunger which can be operated to empty the internal cavity of the tube through the distal end opening. The tube is filled with the appropriate dosage of composition, inserted fully into the vagina, and the plunger is operated to empty it as it is withdrawn therefrom. The length of the applicator ensures that an effective amount of the composition is disposed at the cervical end of the vagina, for maximum protection.

It should be noted that the spermicidal compositions and methods of the invention are intended for mammals. In more specific embodiments, when relating in detail to methods of the present invention the step of administering Fer inhibitors or any composition comprising thereof to a subject should be understood as referring to any member of a mammalian species. By “mammalian subject” is meant any mammal for which the proposed therapy is desired, including human, livestock, equine (that includes horses, asses and zebras), bovine (including domestic cattle, bison, buffalo, the yak, and the four-horned and spiral-horned antelopes), canine, and feline subjects, most specifically humans.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al, (1989); “Current Protocols in Molecular Biology” Volumes I-III Ausubel, R. M., ed. (1994); Ausubel et al, “Current Protocols in Molecular Biology”, John Wiley and Sons, Baltimore, Md. (1989); Perbal, “A Practical Guide to Molecular Cloning”, John Wiley & Sons, New York (1988); Watson et al, “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

All scientific and technical terms used herein have meanings commonly used in the art unless otherwise specified. The definitions provided herein are to facilitate understanding of certain terms used frequently herein and are not meant to limit the scope of the present disclosure.

As used herein the term “about” refers to ±10% The terms “comprises”, “comprising”, “includes”, “including”, “having” and their conjugates mean “including but not limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. As used herein the term “about” refers to ±10%.

The terms “comprises”, “comprising”, “includes”, “including”, “having” and their conjugates mean “including but not limited to”. This term encompasses the terms “consisting of” and “consisting essentially of”. The phrase “consisting essentially of” means that the composition or method may include additional ingredients and/or steps, but only if the additional ingredients and/or steps do not materially alter the basic and novel characteristics of the claimed composition or method. Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

The present invention relates to the treatment of subjects, or patients, in need thereof, as described herein before. It should be noted that specifically in cases of non-human subjects, the method of the invention may be performed using administration via injection, drinking water, feed, spraying, oral gavage and directly into the digestive tract of subjects in need thereof. It should be noted that administering of the Fer inhibitor/s of the invention, specifically, the micellar formulation of E260 or any salt, base, ester or amide thereof, according to the invention to the patient includes both self-administration and administration to the patient by another person.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

### Antibodies

The following primary antibodies were used in various analyses: anti-HA tag (Roche, USA, 1:1000), anti-actin (Santa-Cruz, USA, 1:1000), anti-LC3 (by Sigma-Aldrich, USA, 1:1000), anti-Hexokinase I (HKI) and II (HKII) (Cell signaling, USA, 1:1000), anti-Pyruvate Kinase isozymes M1/M2 (PKM 1/2)(Cell signaling, USA, 1:1000), anti-Phosphofructokinase-1 (PFK1) (Cell signaling 1:500), anti-Lactate Dehydrogenase (LDH) (Cell signaling, USA, 1:1000), anti-Tubulin (Abcam, USA, 1:1000), anti-Poly [ADP-Ribose] Polymerase 1 (Santa-Cruz, USA, 1:1000), anti-Poly-ADP-Ribosylation (PolyADPr) (Alexis, USA, 1:1000), anti-Fer (Sigma-Aldrich, USA, 1:500), anti Fer SH2 (produced by our laboratory, 1:500), anti-Fer N′ terminus (produced by our laboratory, 1:500), anti-Fer C′ terminus (produced by our laboratory, 1:500), anti-AMP-activated protein kinase (AMPK) (Cell signaling, USA, 1:1000), anti-p-AMPK (Cell Signaling, USA, 1:500), and anti-phospho-Tyrosine (anti-pY-Exalpha, USA, 1:500), anti-mammalian target of rapamycin (mTOR) (Cell signaling, USA, 1:100), anti-p-mTOR (Cell signaling, USA, 1:1000), anti-Akt1/2/3 (Santa-Cruz, USA, 1:1000), anti-p-Akt1/2/3 (Santa-Cruz, USA, 1:1000), anti-Regulatory-associated protein of mTOR (Raptor)(Cell signaling, USA, 1:1000), anti-p-Raptor ser722 (Merck-Millipore, Ireland, 1:1000), anti-p-Raptor ser792 (Cell Signaling, USA, 1:1000), anti-phosphoglycerate kinase 1 (PGK) (Abcam, USA, 1:2000), and anti-Caspase 3 (Cell signaling, USA, 1:1000) antibodies.

### Tissues Culture Studies

Cell lines (HCT116, ht29, Rko, Hep3B, SW48, SW620, MDA-MB-231, PANC-1, su.86.86, Hfb) were obtained from the American Type Culture Collection (ATCC). Cell type authentication was carried out by microscope and DNA profiles using short tandem repeat analysis. Cells were grown in Minimum Essential Medium (MEM) containing 10% heat-inactivated Fetal Bovine Serum (FBS) or in Dulbecco's Modified Eagle's Medium (DMEM) containing 10% FBS, where specified, at 37° C. with 5% CO₂. In general, the in-vitro experiments were carried out with 2. μM 0260 added into the growth medium. Dose response experiments included increasing 0260 concentrations. Cell death levels were determined using the MultiTox-Fluor Multiplex Cytotoxicity Assay (G9201, Promega, USA). In brief, 10,000 cells were seeded in black bottom 96 wells plate and were allowed to attach to the plate bottom for 16 h before the administration of various 0260 concentration in each well. Cell death levels were determined according to the manufacturer's protocol.

### Experimental Procedures

### Development of the Micellar 0260 Formulation

Emulsification of 0260 was performed in Cremophor® EL (1-25%) mixed with ethanol (1-25%) and PBS using a combination of CPI and phase PIT emulsification methods. The diameter and volume distribution of micelles containing 0260 were determined using DLS.

### Dynamic Light Scattering (DLS) Measurements of 0260 Containing Micelles

Emulsification of 0260 was performed in Cremophor EL and PBS mixture. Photon Cross-correlation Spectroscopy measurements combined with Pade-Laplace analysis was carried out and the size distribution and stability of the micelles were determined. All analyses were carried out using NanoPhox (Sympatec, Germany).

### Computational Modeling of the Docking of 0260 in Fer/FerT

Homology modeling of a Fer fragment extending from residues 447-820 (as denoted by SEQ ID NO. 7, including the enzymes SH2 (Src Homology dimain 2) and KD (kinase domain), was performed using the MODELER protocol in Discovery Studio version 4.0 (Accelrys Inc, San Diego, USA). As a template, the inventors used the structure of the corresponding domains in the Tyrosine-protein kinase Fes/Fps, PDB 3BKB chain A, with 66% identity, 80% similarity and 1% gaps. Docking of 0260 to Fer/FerT was performed using AutoDock Vina with default parameters. The binding site was defined as the whole protein structure, and 20 binding modes were suggested (scores ranging from 6 to 7.8). The analysis used the highest scoring pose (score=7.8) and the second best pose (score=7.3) positioned in the same cavity.

### Plasmid Construction

The Fer/FerT Kinase Domain (KD) (residues 541-822, as denoted by SEQ ID NO. 8, encoding fragment was cloned into the pRSFDuet-1 expression plasmid, and transformed into E.Coli BL-21, strain.

The following primers were used for Fer KD:

Forward-5′GCCGCGAATTCGAAATCAGGTGTAGTTCTGCT3′, as
denoted by SEQ ID NO. 9;
Backward-5′ CGACTGCGGCCGCCTATGTGAGTTTTCTCTTGAT 3′
as denoted by SEQ ID NO. 10.

### In-Vitro Kinase Inhibition Assay

The Fer/FerT KD protein fragment was expressed in bacterial cells as described above, purified twice using the Talone cobalt affinity resin beads (Clontech, USA) and eluted using a 10-400 mM imidazole gradient. Protein was further purified using a 5000 Dalton cut-off dialysis and concentrated using a 10,000 Dalton cut-off Amicon Ultra-4 centrifugal filter (Merck-Milipore, Ireland). A sample of the purified protein was separated onto SDS-PAGE and subjected to a Wester blot (WB) analysis using specific antibodies directed towards the Fer/FerT KD, to verify the integrity of the purified protein. To test the effect of 0260 on the Fer/FerT KD autophosphorylation activity, 0.5 μg of the Fer/FerT KD protein was incubated in 0.5 ml kinase activity buffer (50 mM HEPES pH 7.5, 10 mM MgCl₂, 1 mM EGTA, 0.01% Brij-35) and 1 μM Adenosine-Tri-phosphate (ATP) (Fermentas, USA). As a negative control, the KD protein was incubated in the same buffer without ATP. The KD and ATP containing mixture was incubated for 1 hour at room temperature with ascending concentrations of E260 dissolved in DMSO or with DMSO alone. Following the incubation period, a sample from the incubated mixture was separated by SDS-PAGE and a WB analysis was performed using specific anti-Fer and anti-pY antibodies to evaluate the inhibitory effect of 0260, as reflected by the diminished phosphorylation level of the Fer/FerT KD.

### Cell Survival Assay

Cells death level was determined using the MultiTox-Fluor Multiplex Cytotoxicity Assay (G9201, Promega, USA) according to the manufacturer's protocol.

### Animal Xenograph Studies

All animal experiments were performed according to the guidelines of the Institutional Animal Care and Use Committee of the Bar-Ilan University. Nude mice (Harlan, Israel) were transferred from ad libitum diet to a more strict diet 2 days before inoculation to lower blood glucose levels, and housed one per cage to ensure an even consumption of food. The diet was comprised of 3 g/mouse/day of standard chow, given at the same time every day. The food was consumed within an average time of 2 hours, consequently the mice were kept without food for the next 22 hours until the daily ration. The mice were kept on this diet throughout the experiment. Mice were inoculated with 1.5×10⁶ SW620 CC cells and divided randomly into respective experimental groups. In brief, cells were harvested, counted under the microscope, concentrated by centrifugation (5 min at 500G) and re-suspended in Hank's balanced salt solution (Biological Industries, Israel). Before inoculation the cell suspension was mixed with Martial (BD biosciences, USA) in 1:1 ratio. Four days post-inoculation mice were randomized and placed in cages (n=4 per cage). Mice were injected intra-peritonealy every 12 h for 22 days with 25 mg/kg or 50 mg/kg of the micellar and non-micellar 0260 formulations, and control mice—with empty micelles or vehicles. Mice were sacrificed at day 22, tissues from relevant organs were fixed in paraffin, sectioned (7 μm sections) and stained with Hematoxylin and Eosin stain (H&E stain) for future histopathological evaluation. Blood samples were drawn from sacrificed mice.

### Pharmaco-Kinetic (PK) Study of 0260

The investigation of PK properties of 0260 was carried out using 30 ICR/CD-1 mice. Intraperitoneal (IP) administration was used, and the solution was administered as 10 ml per 1 kg of animal body weight. At 5, 15, 30, 60, 120, and 240 min after administration (5 mice per each time point) mice were decapitated and blood samples were taken in EDTA-containing tubes. Plasma was prepared from the collected blood by centrifugation and then forwarded for LC-MS/MS analysis. A high sensitivity analytical method was developed for detection of 0260. Analysis was performed in the Agilent 1100 HPLC system including degasser, the binary pump, autosampler, and absorption detector with variable wavelength ranging between 190-600 nm. The HPLC system was coupled with the tandem mass spectrometer API 2000 (Applied Biosystems). The TurboIonSpray ion source was used in the positive ion mode. Data acquisition was performed with Analyst 1.3.1 software.

### Toxicological and Histopathological Studies

Mice were sacrificed at day 22 of the xenograft experiments, as described above. Tissues from relevant organs and tumors were fixed in paraffin, sectioned (7 μm sections) and stained with Hematoxylin and Eosin stain (H&E stain) for histopathological evaluation as previously described. Blood samples were drawn by cardiac puncture. The blood was allowed to clot for 30 minutes at room temperature and serum separation was carried out by 20 minutes centrifugation at 1000 g. The serum was analyzed immediately using the Cobas 6000-501 chemistry analyzer (Roche Diagnostics, USA) according to the manufacturer's protocols for each assay.

### Western Blot (WB) Analyses

WB analyses were carried out as previously described before [4]. In brief, cells were lysed in RIPA buffer mixed with protease inhibitors and separated on SDS-PAGE gel, transferred to PVDF membrane treated with 5% skim milk to exclude non-specific background and incubated with a primary antibody over-night. The membrane was incubated for 1 h with a secondary antibody conjugated to Horseradish Peroxidase (HRP). The protein-antibody complex was visualized using the SuperSignal West Pico Chemiluminescent Substrate (Thermo scientific, USA).

### Immunoprecipitation (IP) Analyses

Protein lysates (1 mg) were prepared from tissues of adult mice according to previously described protocol [4], including incubation with Protein A Sepharose (GE Healthcare) to avoid nonspecific binding. Protein lysates were incubated with specific antibodies diluted 1:100 overnight at 4° C. Antigen-antibody complexes were precipitated with Protein A Sepharose, washed twice in PBS and eluted in Sample Buffer (2% SDS, 1% DTT 0.02% bromophenol blue 10% glycerol, 12.5 mM EDTA, 50 mM Tris-HCl pH 6.8) at room temperature for 10 min and at 100° C. for 8 min. Eluates were separated on SDS-PAGE and visualized by WB using specific antibodies.

### Ethidium Homo-Dimer III (EthD III) Incorporation Assay

Onset of necrosis was monitored using the Ethidium homo-dimer III (EthD III) incorporation assay, according to THE manufacturer's protocol (Biotium, USA). EthD III staining in necrotic cells or Hoechst staining in live intact cell were visualized immediately by Axioimager z1 fluorescent microscope (Zeiss, German).

### Assay for Mitochondrial Membrane Potential (MMP)

For microscopic and ELISA based analyses of cells MMP, Tetra-Methyl-Rhodamine-Ethyl ester (TMRE) incorporation assay was carried out according to the manufacturer's instructions (Abcam, USA).

### Assay for Electron Transport Chain (ETC) Complex I Activity

SW620 cells were treated with 0260 for 4-22 h and the Complex I activity was determined for each period on incubation using the Complex I Enzyme Activity Microplate Assay Kit (Colorimetric) according to the manufacturer's instructions (ab109721).

Terminal Deoxynucleotidyl Transferase dUTP Nick End Labeling (TUNEL) Assay

Samples of 1×10⁶ cells were inoculated into a 6 well plate containing a cover slip, and fixed for 30 min with 4% paraformaldehyde following adhesion. The cells were then subjected to TUNEL analysis according to manufacturer's instructions (Roche, USA).

### High Pressure Liquid Chromatography (HPLC) for ATP Quantification

Quantification of intracellular ATP was carried out using HPLC as previously described.

### Fluorescence Microscopy and Immunocytochemical (ICC) Analyses

Cells were fixed with 4% paraformaldehyde (30 min) and incubated with Permeabilization Solution containing 0.1% Triton X-100 (Sigma-Aldrich, Israel) in PBS at room temperature for 1 h. Nonspecific binding was blocked with PBS containing 10% fetal bovine serum for 1 h at room temperature. Cells were incubated with specific antibodies diluted 1:100 overnight at 4° C. Cells were washed (5 min) in PBS containing 0.2% Tween-20 (Sigma-Aldrich, Israel) to remove non-specific binding. Bound primary antibodies were detected by Alexa Fluor® 488 goat anti-mouse IgG antibody or Alexa Fluor® 594 goat anti-rabbit IgG antibody (Molecular Probes, Invitrogen, U.K.). Nuclei were visualized by incubation with Hoechst 33342 solution for 10 min (1:500). Nonspecific staining, as determined by binding to the secondary antibody only, was negligible. The immunostained slides were examined under the fluorescence microscope AxioimagerZ1 (Zeiss, Germany) or confocal fluorescence microscope (Olympus-FV1000).

### Interactome Analysis

Interactome analysis of Fer kinase was carried out as previously described [9]. Briefly, Fer was immuno-precipitated from SW620 CC cells, followed by protein separation on SDS PAGE and LC-MS/MS analyses.

### Transmission Electron Microscopy (TEM)

Cells were grown in MEM or DMEM, washed twice with PBS and fixed for 5 minutes in Karnovsky fixative (2.5% glutaraldehyde, 2.5% paraformaldehyde in cacodylate buffer pH 7.4) on the tissue culture plate. Cells were then harvested, and transferred to 1.5 ml tube for further 1 h fixation at room temperature. The cells were then stored O.N at 4C° after which the samples were washed in 0.1 M cacodylate buffer and post-fixed with 1% OsO4 in 0.1 M sodium cacodylate buffer (Sigma-Aldrich, Israel) for 1 h. Samples were then dehydrated in alcohol and propylene oxide followed by embedding in Agar Mix. Thin sections (60 nm) were cut, stained with uranyl acetate and lead citrate, and observed in a FEI Tecnai transmission electron microscope.

### ATP Levels Analysis

Cells were suspended in 400 μl cold 0.4M perchloric acid solution, incubated on ice for 15 min, centrifuged at 12,000×g, neutralized with 4M K₂CO₃ to pH 7.5, incubated for 15 min on ice and concentrated (10,000×g). Chromatographic analyses were performed using Hitachi Elite LaChrom system at 30° C. applying supernatants to SUPELCOSIL™ LC-18-S HPLC Column (5 μm particle size, L×I.D. 25 cm×2.1 mm), flow rate 0.2 mL/min under isocratic elution conditions (50 mM potassium phosphate, 100 mM triethylamine, pH 6.5 (adjusted with phosphoric acid)):Acetonitrile (98.5:1.5)). Each analysis cycle was set to 30 min, chromatographic flow was monitored at 260 nm and integrated using EZChrom Elite Software.

### Quantification of Glycolysis Rate

Cells were grown in MEM containing 1 gr/lit glucose or in DMEM with 4.5 gr/lit glucose. Secreted lactate levels were determined using Lactate Colorimetric Assay Kit II according to the manufacturer's protocol (Biovision, USA).

siRNA Mediated Silencing of Fer

siRNA mediated silencing of Fer in SW620 cells was carried out using Lipofectamine2000 (Invitrogen, USA), using the following siRNA sequences:

1. siRNA-fer 5′ ACGUAUCCAAGUCUUGGCUACUUAU 3′, as
denoted by SEQ ID NO. 11.
2. siRNA-fer 5′ GGAAUUACGGUUACUGGAAACAGUA 3′, as
denoted by SEQ ID NO. 12.

3. For negative control (siRNA-neg)—Stealth RNAi siRNA Negative Control Med GC (Invitrogen, USA).

### Sperm Cells Extraction

For isolation of spermatogenic cells, mice testes were removed from out-bred (ICR) mice and digested in Isolation Medium (DMEM with 2 mM glutamine, 1.5 mM sodium pyruvate, 10% fetal calf serum, 25 μg/ml ampicillin and 1× non-essential amino acids) with added 2 mg/ml collagenase (Sigma-Aldrich) for 5 min at 37° C., and washed twice. Extracellular DNA and proteins were removed with 1 unit/ml DNAse and 0.3 ml 0.5% trypsin at 37° C. for 5 min. Cells were filtered through a nylon mesh with 50 μm pores and incubated in Isolation Medium with 0260 or control for 24 h, and examined under the microscope. Mouse mature caudal spermatozoa were obtained by dissecting the cauda epididymis of male ICR mice and puncturing them in a dish containing 3 ml PBS to enable sperm release out of the epididymal tissue. The sperm suspension was filtered through a nylon mesh with 50 μm pores, centrifuged at 1000×g for 5 min and re-suspended in a buffer compatible with the experimental procedure.

### Sperm Cell Motility Assay

Sperm cell motility was evaluated using the Computer-Aided Sperm Analysis (CASA). Samples (5 μl) were placed in standard count four chamber slide (Leja, Nieuw-Vennet, Netherlands) and analyzed by CASA device with IVOS software (version 12, Hamilton-Thorne Biosciences), acquiring up to ten sequels, 10 s long, for each sample, using parameters identifying mouse sperm motility. The proportion of hyper-activated (progressive motility) spermatozoa in each sample was determined using the SORT function of the CASA instrument. HAM was defined by curvilinear velocity (VCL)>90 μm/s, linearity (LIN) b20% and an amplitude of lateral head (ALH)>7 μm.

### Kinase Panel Assay

Kinase panel assays were performed using the FRET-based Z′-LYTE, Lantha Screen and Adapta enzymatic kinase assay systems (Life Technologies, Carlsbad, Calif.), which were carried out using the SelectScreen Kinase Profiling Service (Life Technologies). The standard reaction contained 2 μM peptide substrate, ATP at the optimal assay concentration, 50 mM HEPES (pH 7.5), 0.01% Brij-35, 10 mM MgCl₂, 1 mM EGTA, and the tested kinase. Each kinase was checked for its activity in duplicate data points (n=2) and was assayed following the detailed procedures outlined in the SelectScreen service. Percent inhibition or ATP displacement (For Lantha screen) represent a ratio of phosphorylated/ATP bound product formed in the presence of the compound compared with phosphorylated/ATP bound product formed in reactions containing 1% DMSO. Inhibition or ATP displacement data indicate the mean value of kinase inhibition from duplicate data.

### Statistical Analysis

Statistical analysis was performed using the paired and unpaired Student's t-tests, with a p value<0.05 being considered significant. Results are depicted as mean±standard deviation (+/−SD) or ±standard error (+/−SE) of the mean for n given samples. For statistical analysis of groups with multiple comparisons, one way ANOVA analysis was performed with Bonferroni post-hoc, with p value<0.05 considered significant.

### Example 1

### Developing 0260 Micellar Formulation and Evaluating its Efficiency in Malignant Cells

0260 was incorporated into micelles formed in the presence of Cremophor® EL (1-25%) mixed with ethanol (1-25%) and PBS using a combination of CPI and PIT emulsification methods to achieve the most optimal and effective formulation of 0260 for in-vitro and in-vivo studies. Formation and diameter of obtained micelles was evaluated using DLS. Average micelle diameter was established under various conditions of Cremophor EL/ethanol/PBS proportions and CPI±PIT, relating to the maximal diameter of 16 mn as maximal micelle loading. Optimal conditions for producing >95% micelles with 16 mn or more were established after numerous trials (FIG. 1).

The 0260 micellar formulation when applied to SW620 CC proved to be significantly more efficient cytotoxic agent than the non-micellar formulation. EC50 after 24 h treatment was 0.35 μM for the micellar compared to 6 μM for the non-micellar formulation (calculated from slope), and after 48 h-0.2 μM compared to 3.35 μM, respectively (FIGS. 2A-2B).

Further, the 0260 micellar formulation proved to be more efficient in attenuating progression of SW620 CC xenografts in nude mice (FIGS. 3A-3B). Specifically, intraperitoneal (IP) administration of 25 mg/Kg of the 0260 micellar formulation was 15 times more efficient (dashed line) than the non-micellar formulation administered at the same dose (line with rectangle markers) and 5 times more efficient than the non-micellar formulation at a twice higher dose of 50 mg/Kg (line with circle markers).

Biological effects of the micellar 0260 formulation, as determined for example by the above assays in vitro and in vivo, were preserved after prolonged storage of more than two months.

The following EXAMPLES apply to 0260 micellar formulation of the invention.

### Example 2

### Targeting 0260-Fer Interaction to the Fer Kinase Domain

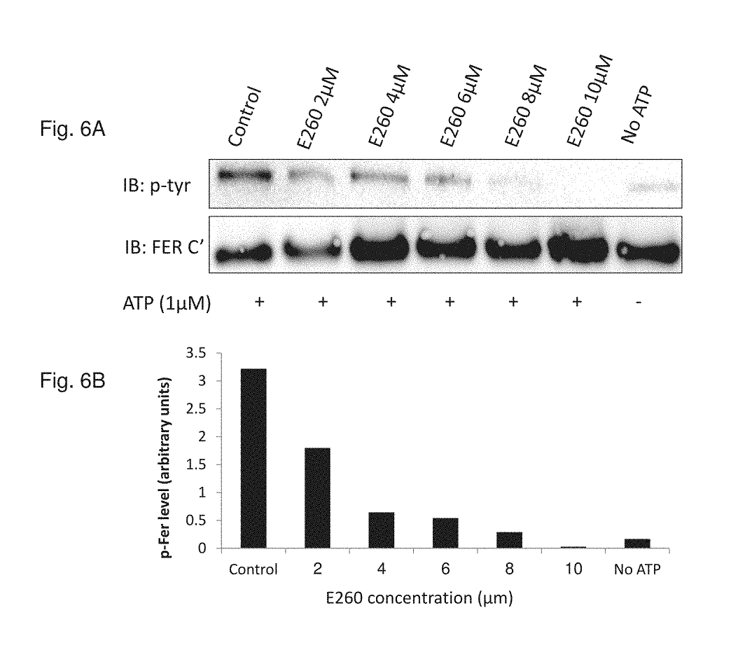

To examine the effect of 0260 micellar formulation on Fer in malignant cells, the kinase was immuno-precipitated from untreated and from 0260 treated SW620 CC cells. While 0260 did not disable the IP of Fer by antibodies directed toward the N-terminal tail of the protein, it did disable the immuno-precipitation of Fer by antibodies directed toward the enzyme's C-terminal-KD (FIGS. 4A-4B), thereby substantiating the notion that 0260 binds the Fer C-terminal portion.

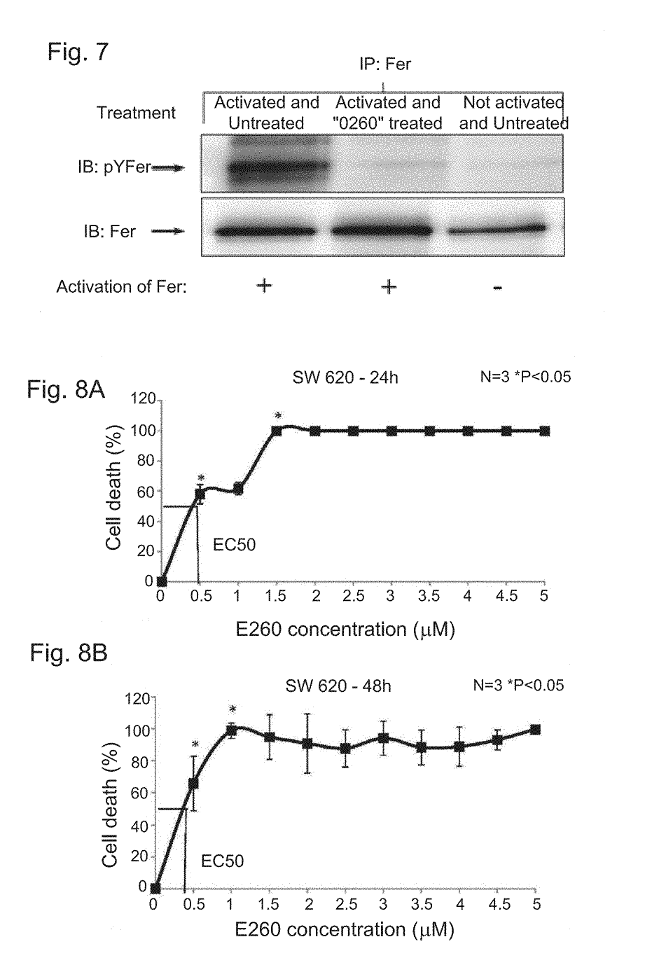

Computational analysis of 0260 docking in the modeled whole Fer protein revealed that the highest scored binding mode of 0260 to Fer falls in the ATP binding pocket of the enzyme's KD (FIGS. 5A-5C). Specifically, homology modeling of the Fer C-terminus, residues 447-820 including SH2 and KD domains, was performed using MODELER protocol in Discovery Studio version 4.0 (Accelrys Inc, San Diego, USA) on the basis of template structure of corresponding Fes/Fps KD domains, PDB 3BKB chain A, with 66% identity, 80% similarity and 1% gaps. 0260 docking to Fer was evaluated using AutoDock Vina (PMID: 19499576) with default parameters, relating to a binding site as the whole structure. Twenty binding modes were suggested (scores ranging from 6 to 7.8). Using the highest scoring pose (score=7.8), the analysis yielded a putative 0260 binding site. Notably the second best pose (score=7.3) was also positioned in the same cavity.

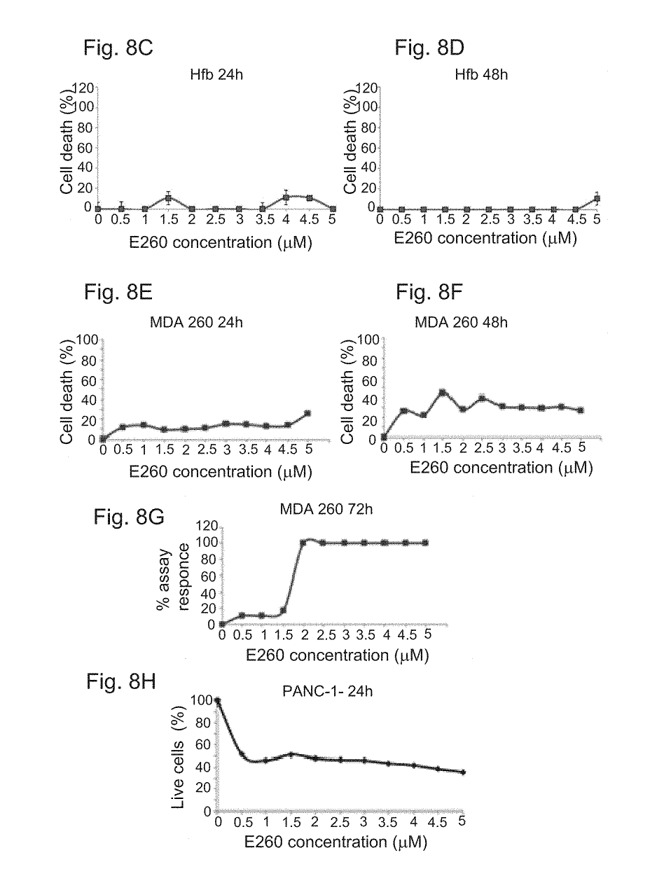

To demonstrate that 0260 directly targets Fer and FerT, an in-vitro kinase assay was performed using a purified KD-containing fragment of these enzymes. This analysis demonstrated the direct inhibitory effect of 0260 on this domain as reflected by the significantly decreased auto-phosphorylation level of the Fer/FerT KD when incubated with 1 μM ATP and increasing concentrations of 0260 (FIG. 6A, 6B).

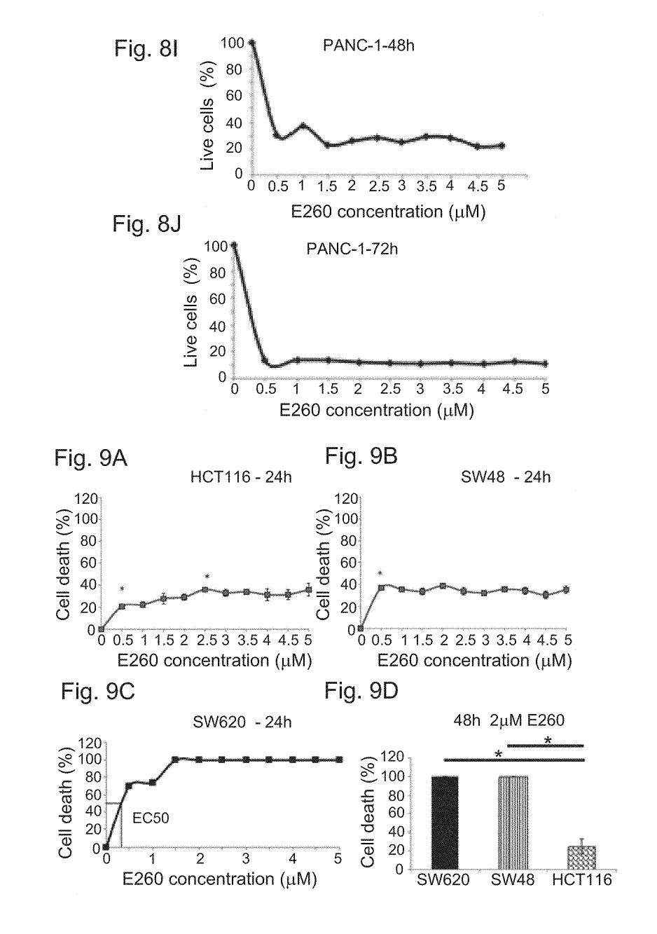

The inhibitory effect of 0260 on Fer autophosphorylation level was further demonstrated in RKO CC cells treated with 0260 for 24 h, using WB analysis of activated Fer IP from lysates of control and CC cells and anti-phosphotyrosine antibody (FIG. 7).

### Example 3

### 0260 Selectively Invokes Death in Malignant but not in Normal Human Cells

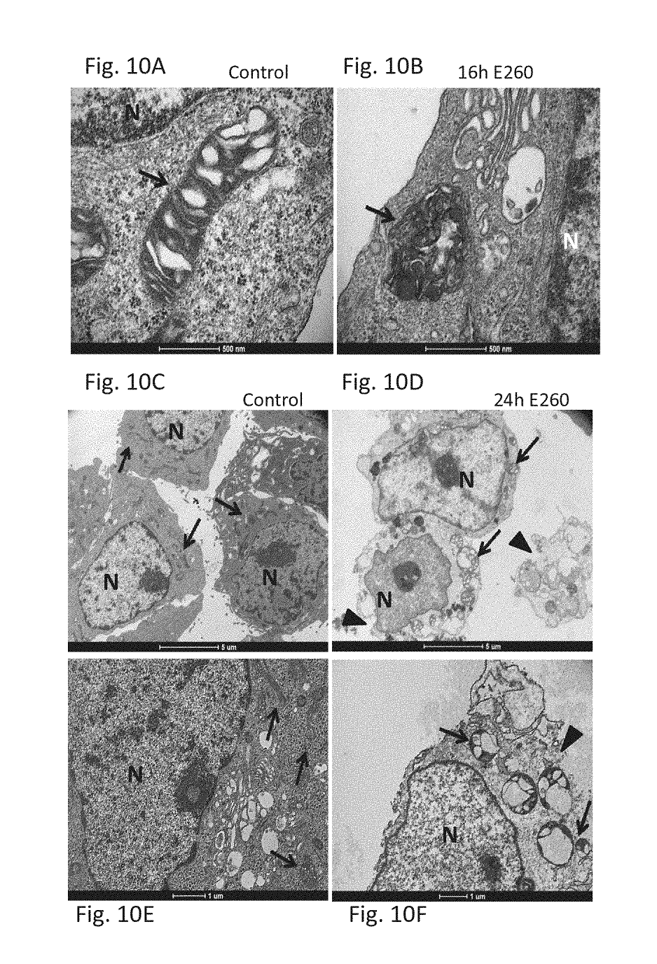

To examine specificity of the 0260 micellar formulation to malignant cells, primary Hfb, SW620 CC, PANC-1 and MDA cells were exposed to increasing 0260 concentrations for 24 h, 48 h and 72 h (MDA and PANC-1 only). The proportion of cell death, as determined by MultiTox-fluorescence multiplex cytoxicity assay, clearly indicated that 0260 selectively induced cell death in malignant and not normal human cells (FIGS. 8A-8J).

More specifically, to characterize the effect of 0260 in malignant cells, metastatic grade IV SW620 CC cells were treated with 0260 followed by analysis of viability. Onset of death was observed in the 0260 treated cells, with an EC50 value of 400 nM after 24 h of treatment and an EC50 of 300 nM after 48 h (FIGS. 8A-8B). Importantly no death was seen in normal human fibroblasts (Hfb) which were treated with 0260 under the same conditions (FIGS. 8C-8D). In two cell-lines, MDA and PANC-1, derived from breast and pancreatic carcinomas (FIGS. 8E-8G and 8H-8J, respectively), 0260 was also found efficient in inducing cell death at concentrations of 1.5 μM and less.

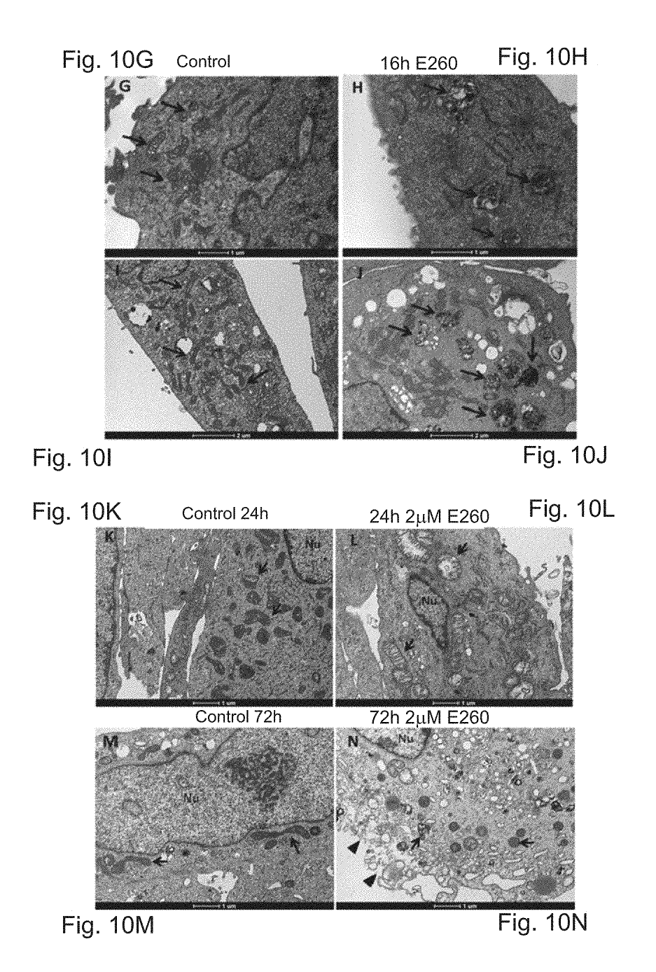

To examine if the 0260 effect cells with various degree of malignancy, rate of 0260-induced cell death was compared in CC cells representing Stage I, Stage IV and metastatic cancers (FIGS. 9A-9D). This experiment demonstrated that 2 μM 0260 micellar formulation induced almost 100% cell death after 24 h treatment in metastatic CC (FIG. 9C), and after 48 h in both metastatic and stage IV CC (FIG. 9D).

### Example 4

### E260 Selectively Evokes Mitochondria Deformation and Necrotic Death in Malignant Cells

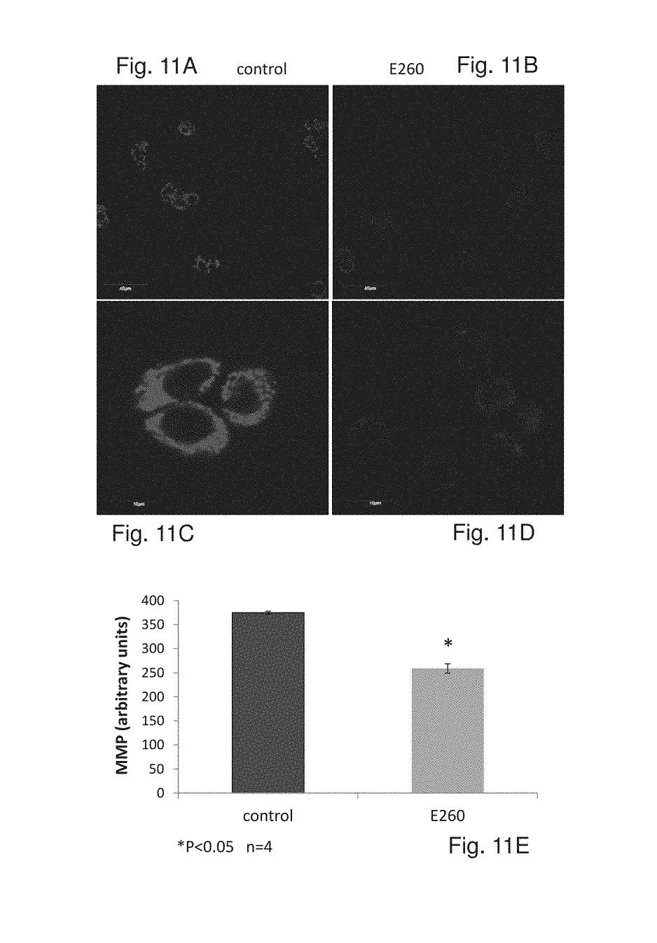

To characterize the death process imposed by 0260 in the treated malignant cells, a TEM analysis was performed on SW620 cells treated with 0260. This revealed the deformation and loss of the normal elongated shape of mitochondria in cells treated with E260 for 16 h (FIGS. 10A-10N). Close examination revealed that the cristae of the damaged mitochondria collapsed and the mitochondria swelled (FIG. 10B). After 24 h of treatment, the mitochondria underwent lysis, and, while integrity of the nuclei was preserved, disruption of the cytoplasmic membrane of the treated cells could be seen in the treated cells (FIGS. 10D and 10F) but not in the vehicle treated control cells (FIGS. 10C and 10E). Disintegration of the plasma membrane accompanied by preserved integrity of the cell nucleus indicated the onset of necrotic rather than apoptotic death in the 0260 treated cells. Analogous effects of 0260 on cytoplasmic membrane were observed in MDA-MB-231 and PANC-1 treated cells (FIGS. 10G-10H).

Destruction of the mitochondria was accompanied by depolarization of the Mitochondrial Membrane Potential (MMP) in 0260 treated but not in vehicle control malignant cells (FIGS. 11A-11E), an effect that absent in 0260 treated normal human cells (FIGS. 11F-11J). Uptake of EthD-III by the affected cells, a hallmark of necrotic death, was seen in the E260 treated cells (FIGS. 11K-11N). This was consistent with the TEM analysis, indicating that necrotic rather than apoptotic death is evoked by 0260 in CC cells.

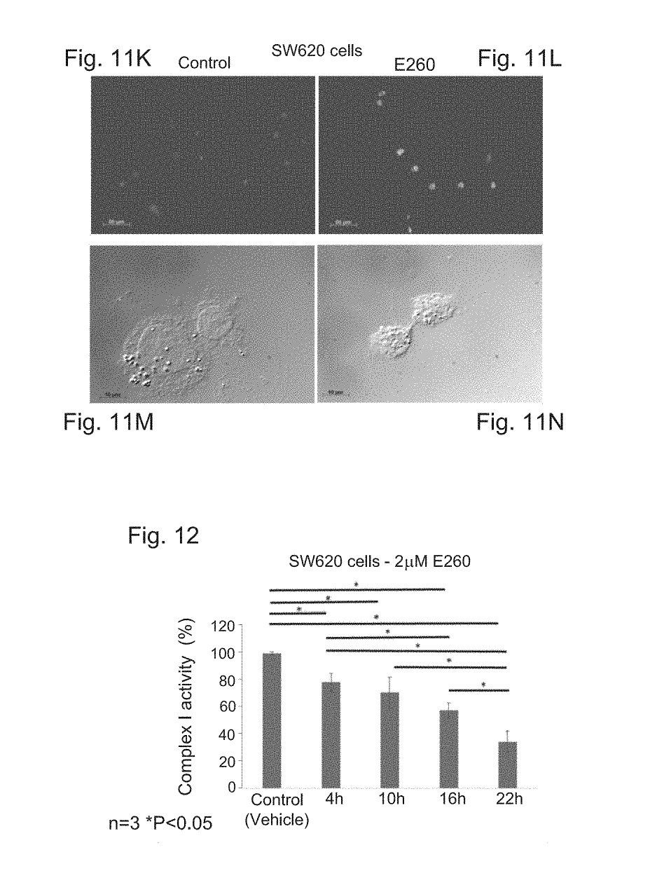

The effect of 0260 on mitochondria was further evaluated regarding the Electron Transport Chain (ETC) complex I activity. Notably, Complex I activity was inhibited by 0260 in SW620 cells as early as 4 h post treatment and was reduced to around 55% activity after 16 h and 35% activity after 22 hours (FIG. 12). Both Fer and FerT naturally associate with mitochondria of cancer cells and are bound to complex I of the ETC, where they support its function and activity. Findings that inhibition of Fer/FerT kinase activity by 0260 significantly decreased Complex I activity suggest that 0260 compromises mitochondrial functionality and integrity by inhibiting Fer/FerT and thereby Complex I activity.

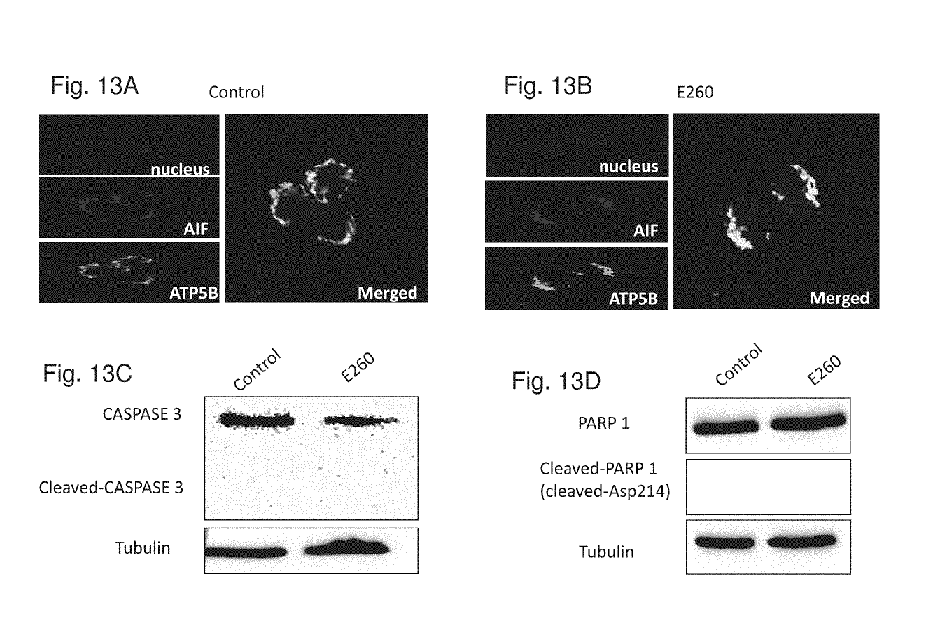

To corroborate the lack of apoptosis in 0260-induced cell death, the appearance of apoptotic markers including cleaved caspase 3, cleaved PARP-1 and translocation of AIF to the nucleus was examined in the treated cells, and these apoptotic hallmarks were not observed (FIGS. 13A-13D).

### Example 5

### 0260 Induces ATP Depletion and Onset of Autophagy in Malignant Cells

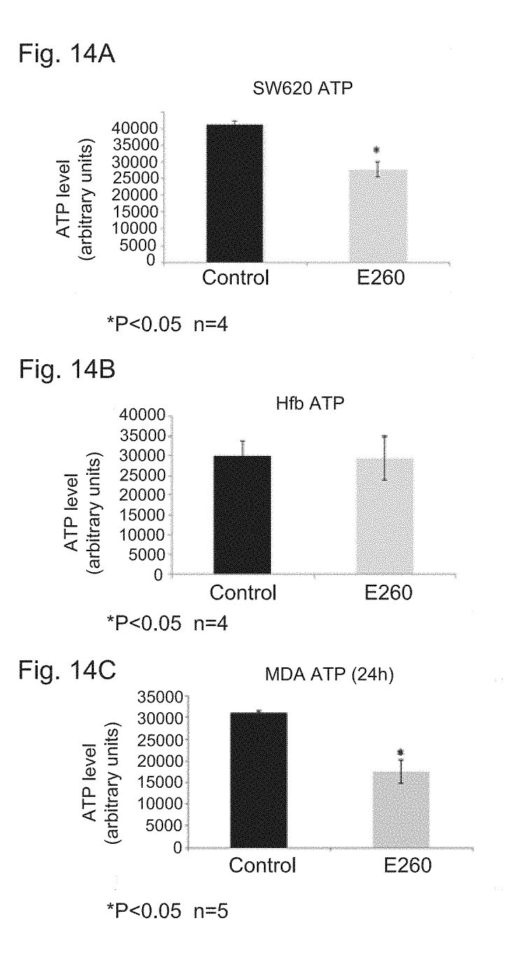

Deformation of mitochondria and reduced MMP suggest a disruption of the energy balance in the 0260 treated malignant cells. To support this finding, the cellular ATP content was determined in 0260 treated malignant cells (CC and MDA cells) and control (Hfb). A decrease of 35%-40% was observed in the cellular ATP level of malignant cells treated with 0260 for 12 hours, a time point preceding the onset of death (FIGS. 14A, 14C). Notably, this effect of 0260 on the cellular ATP level was not seen in Hfb after 12 hours of treatment (FIG. 14B).

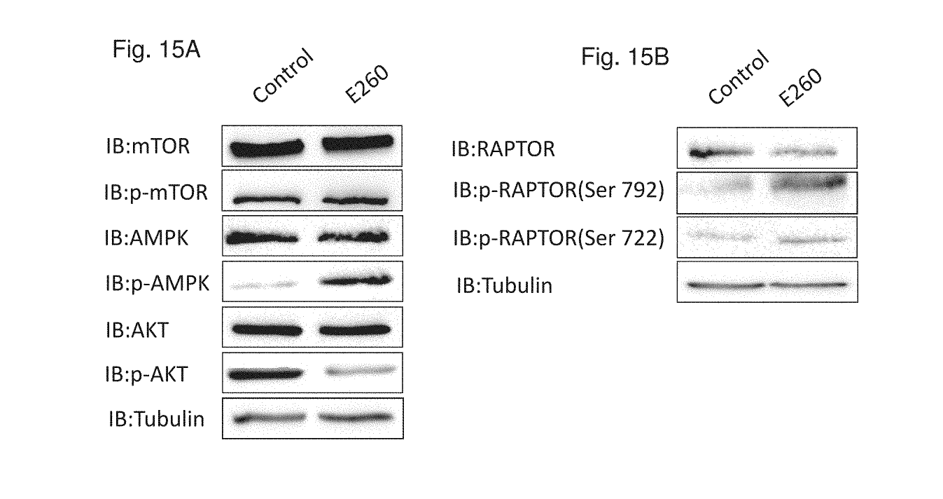

Mitochondrial damage and decrease in the cellular ATP level could direct the initiation of autophagy in 0260 treated CC cells. In accordance with the observed energy deficit, the metabolic sensor adenosine-mono-phosphate protein kinase (AMPK) was phosphorylated and activated in the E260 treated cells. This was accompanied by the inhibitory phosphorylation of the AMPK down-stream effector-Raptor on serines 792 and 722 (FIG. 15B) and by a decrease in the activating phosphorylation of Akt (FIG. 15A). Phosphorylation of Raptor leads to its dissociation from the mTOR complex, thereby leading to inactivation of mTOR kinase activity regardless of its phosphorylation state, which indeed was not changed in the 0260 treated cells (FIG. 15A).

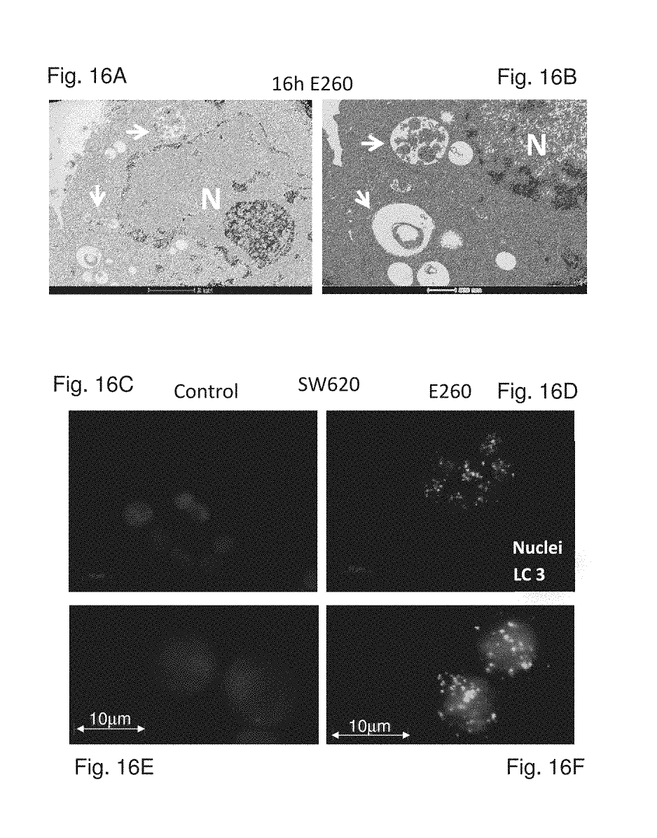

Deactivation of mTOR relieves its inhibitory effect on autophagy, thereby enabling the activation of this process. In accordance with this notion, autophagy was induced and autophagosomes accumulated in 0260 treated malignant cells, as was observed by TEM analysis in CC cells (FIGS. 16A-16B) and immuno-staining for the autophagosome marker-LC3 (FIGS. 16C-16F). Onset of autophagy was also evident from the relative increase in the level of the autophagosome-associated LC3 form—LC3II, in the 0260 treated cells (FIG. 16G). The induction of autophagy was also observed by immuno-staining for the autophagosome marker-LC3 in MDA cells treated with 2 μM 0260 (FIGS. 16H-16I).

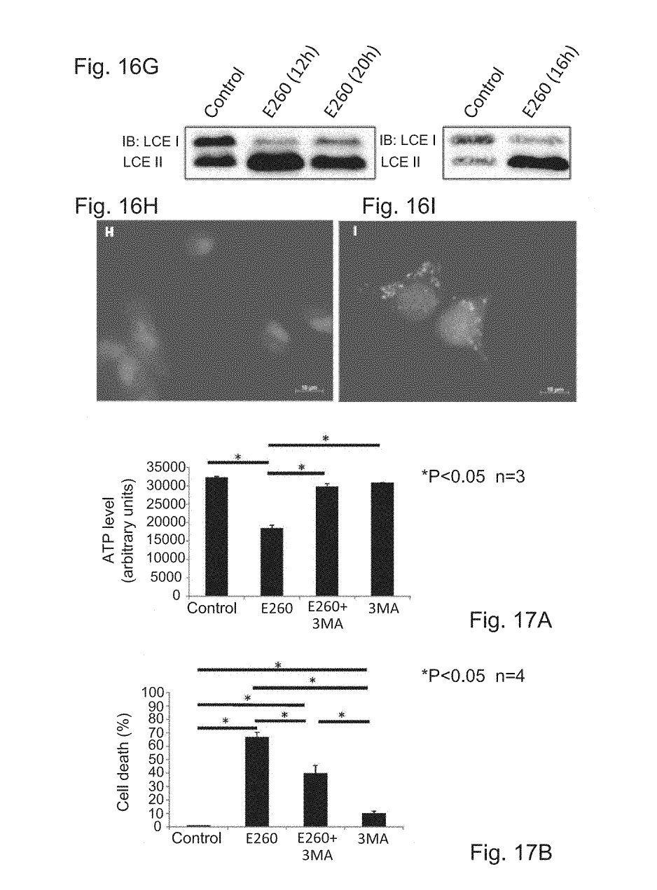

To examine whether the induced autophagy acts as a salvage process, or whether it contributes to the evoked death, SW620 cells were simultaneously treated with both 0260 and the autophagy inhibitor -3-Methyladenine (3-MA). Surprisingly, inhibition of 0260-induced autophagy with 3-MA almost completely restored the ATP level in 0260 treated malignant cells (FIG. 17A). Inhibition of autophagy reduced death levels, although not completely restoring the viability of 0260 treated cells (FIG. 17B). This suggested that the induced and persisting autophagy consumes ATP and contributes to the necrotic death imposed by 0260.

### Example 6

0260 Up-Regulates PARP-1 Activity by Dissociating it from Fer, Thereby Directing Activation of Autophagy and Onset of Cell Death

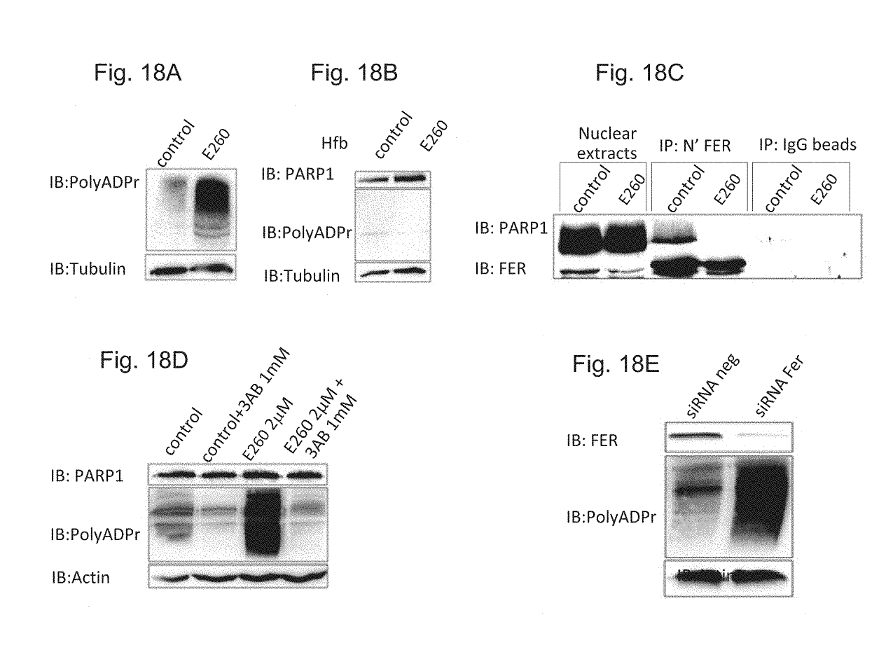

To identify cellular pathways in which Fer is engaged in malignant cells, and proteins that interact with this kinase, a proteomic, Fer-interactome analysis was carried-out. One of the proteins that were repeatedly identified by this analysis was PARP-1 (data not shown). It was hypothesized that 0260 effect on ATP depletion and cell death in malignant cells could result from deregulated activity of the PARP-1 enzyme, which was recently related to depletion of cellular ATP pool [8]. Specifically, lysates from untreated and 0260 treated CC cells were subjected to WB using the anti-poly-ADPr antibody, which revealed induction of poly-ADPr suggestive of up-regulation of PARP-1 activity in 0260 treated CC cells, but not in normal Hfb cells (FIGS. 18A-18B).

Co-immunoprecipitation analysis confirmed the existence of association of Fer with PARP-1 in nuclear lysates prepared from CC SW620 cells (FIG. 18C). Notably, treatment with 0260 dissociated Fer from PARP-1, and the two proteins failed to co-immuno-precipitate from lysates of treated CC cells (FIG. 18C). It was further examined if apart from the dissociating effect of 0260 on the Fer-PARP-1 complex 0260 may also affects PARP-1 activity. Surprisingly, when subjected to 0260 CC (FIG. 18D) but not normal cells (FIG. 18B), exhibited significant increase in the poly-ADP-ribosylation activity of PARP-1. This dysregulated activity was abolished upon exposure of 0260 treated malignant cells to the PARP-1 inhibitor, -3-Aminobenzamide (3AB) (FIG. 18D), thus confirming direct involvement of PARP-1 in this induced poly-ADP-ribosylation activity.

0260-directed dissociation of Fer from PARP-1 and the accompanying induction of PARP-1 activity suggested involvement of Fer in the regulation of the PARP-1 function. This was substantiated in siRNA-directed Fer knock-down experiment also leading to an increase in the PARP-1 ADP-poly-ribosylation activity (FIG. 18E).

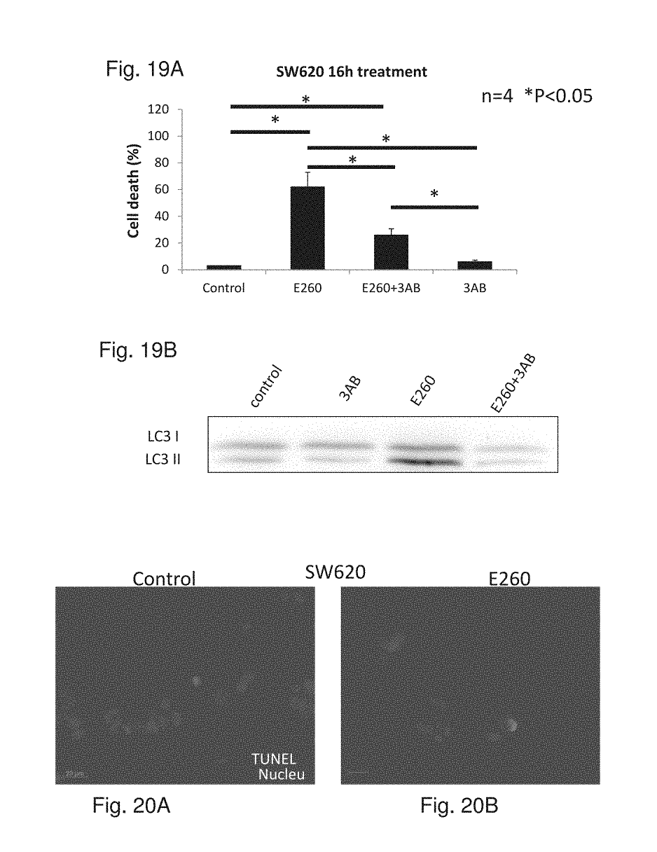

Up-regulated activity of PARP-1 consumes ATP and can drive necrotic death in mammalian cells. To directly examine the involvement of increased PARP-1 activity in 0260-induced cellular death, CC cells were simultaneously subjected to 0260 and to PARP-1 inhibitor, 3AB. The presence of 3AB significantly decreased (by 50%) 0260-induced death in CC cells (FIG. 19A), thus supporting the notion that elevated PARP-1 activity contributes to 0260 cytotoxicity in CC cells. It should be noted that PARP-1 acts as a positive regulator of autophagy, wherein inhibition of PARP-1 activity decreased the level of induced autophagy in 0260 treated cells, as reflected by a decreased LC3II to LC3 I ratio (FIG. 19B).

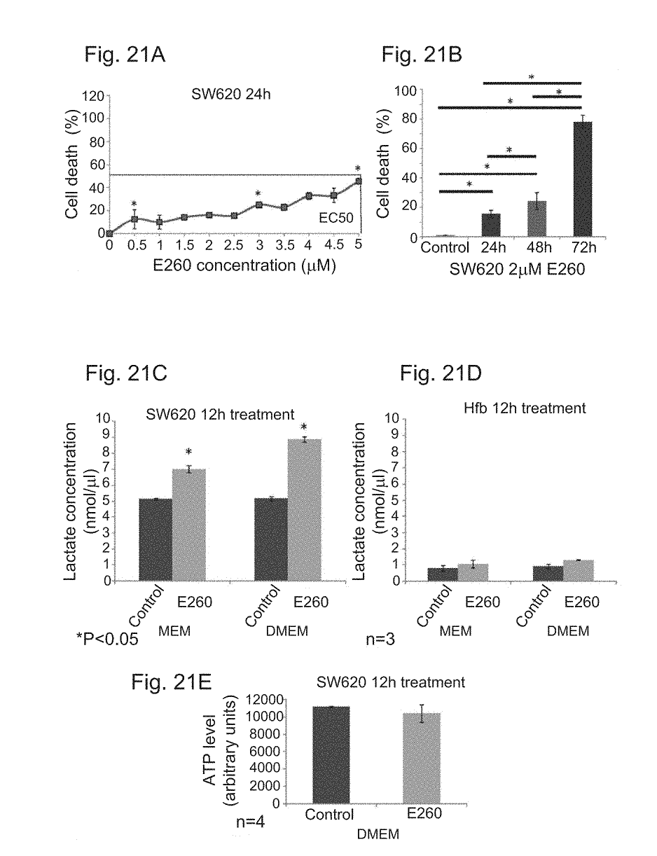

To exclude the possibility that DNA damage induced by 0260 is responsible for activation of PARP-1, SW620 cells were subjected to E260 for 12 h, a time period that precedes cell death, and during which PARP-1 becomes activated. Cells were then subjected to TUNEL analysis, which did not reveal any effect of 0260 on DNA integrity in the treated CC cells (FIG. 20A-20B). These findings support the roles of PARP-1 in the induction of autophagy and cell death in 0260 treated malignant cells.

### Example 7

### High Extracellular Glucose Level Delays the Onset of Death in 0260 Treated CC Cells

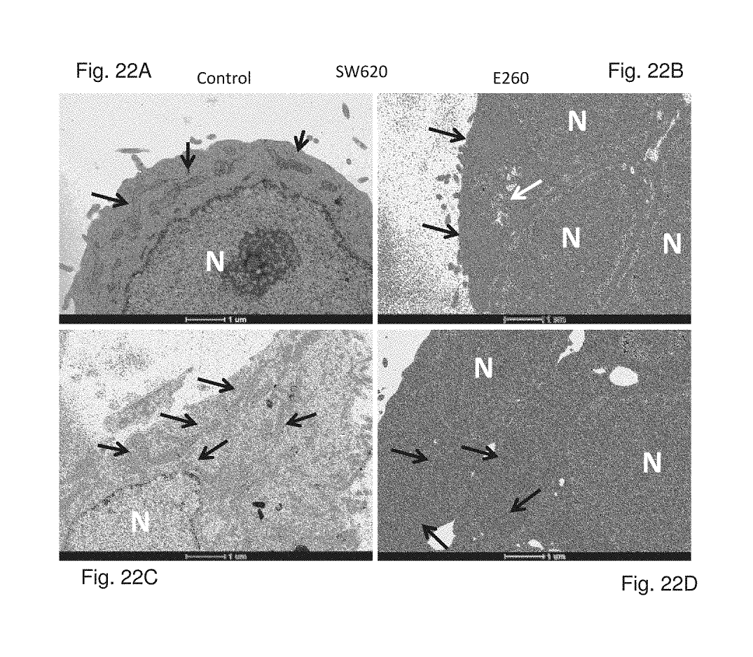

In parallel to their mitochondrial energy production, cancer cells efficiently perform and utilize glycolysis for both energy production and anabolic metabolism. The effect of extracellular glucose concentrations was studied on the 0260 cytotoxicity in SW620 cells. While in previous experiments the presence of 1 gr/1 extracellular glucose and 2 μM 0260 induced 100% cell death in CC cells within 24 h, with EC50 of 400 nM (FIG. 8A), in this experiment under 4.5 gr/1 glucose (DMEM), SW620 CC cells treated with 0260 demonstrated only 50% death at the highest dose with EC50 of 5.1 μM (FIG. 21A). Nevertheless, it should be noted that 100% cell death was still observed when cells grown under high glucose concentration were treated with 2 μM E260 for 72 h (FIG. 21B).

To examine whether delayed death of CC cells treated with 0260 under high-glucose is linked to unregulated glycolysis, the glycolytic rate was compared in untreated and 0260 treated (12 h) cells by measuring concentrations of secreted lactate. This analysis revealed that CC cells accelerate their glycolytic rate by 35% when subjected to 0260 (FIG. 21C). The rate of glycolysis was further accelerated by additional 30% under high glucose concentration resulting in 65% elevation in the glycolytic rate in 0260 treated cells (FIG. 21C). An analogous elevation in the glycolytic rate was not observed in Hfb cells treated with 0260 under low or high glucose (FIG. 21D), further demonstrating selective properties of 0260 in malignant cells. To support the notion that up-regulated glycolysis under high glucose conditions transiently opposes the effect of 0260 on ATP levels in CC cells, cellular ATP levels were quantified in untreated and 0260 treated CC cells. This analysis confirmed that high glucose medium interferes with 0260 activity, reducing its effect on ATP levels in treated CC cells (FIG. 21E).

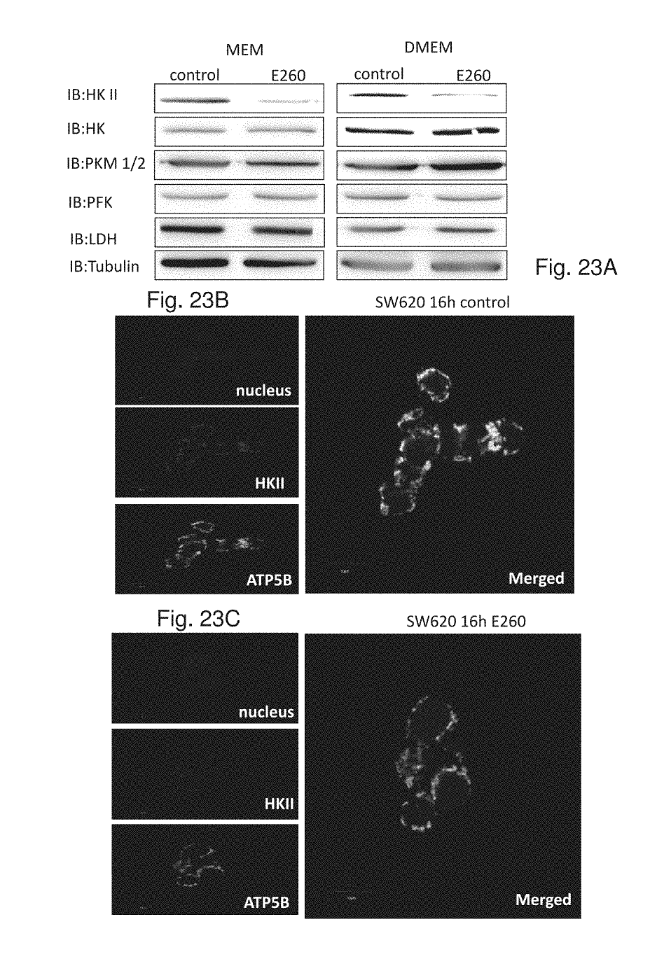

Although death of CC cells subjected to E260 and grown under high glucose conditions was delayed, mitochondrial damage was still observed when these cells were treated with E260 for 24 h and examined by TEM (FIGS. 22A-22D).

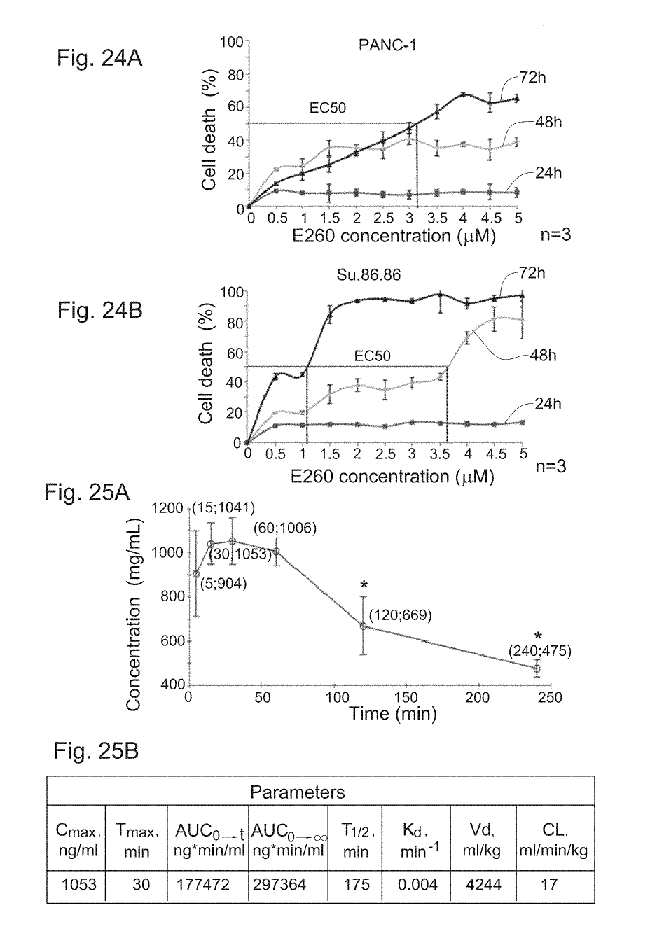

It was further hypothesized that the fact that enhanced glycolysis could not salvage cells from cellular death might be linked to the fact that mitochondrial damage caused by 0260 was accompanied by down-regulation of HKII (FIG. 23A), which associates with the mitochondrial outer membrane and serves as the key initiating enzyme of the glycolytic pathway in cancer cells. Interestingly, neither the level of Hexokinase I, which is the main glycolysis initiating enzyme in normal cells, nor that of other glycolytic enzymes was affected under 0260 treatment (FIG. 23A). The down regulation of HKII was also demonstrated by its decreased presence on the mitochondria of 0260 treated cells (FIGS. 23B-23C).

### Example 8

### Selectivity of 0260-Induced Cytotoxic Effect in Malignant and Normal Human Cells

Selectivity of 0260-induced cytotoxic effect was further examined in normal and malignant cells. Normal human fibroblasts (Hfb) treated with increasing high concentrations (0-20 μM) of 0260 for 24 h, 48 h and 72 h yielded EC50 value of 20 μM at 72 h (not shown). In contrast, SW620 advanced metastatic colon cancer cells treated with increasing concentrations (0-5 μM) of 0260 for 24 h, 48 h yielded EC50 of 400 nM and 300 nM at 24 h and 48 h, respectively (FIG. 8A, 8B). These results show that sensitivity of normal cells to 0260 is 50-fold lower and is achieved only after 72 h of incubation, which is significantly longer than the period required for SW620 cells. Almost no cytotoxic effect was observed in normal after 24 h and 48 h, a period of time in which SW620 cells have reached a complete cell death under relatively low 0260 concentrations. Together, these dose response curves suggest that 0260 may have a relatively large therapeutic window.

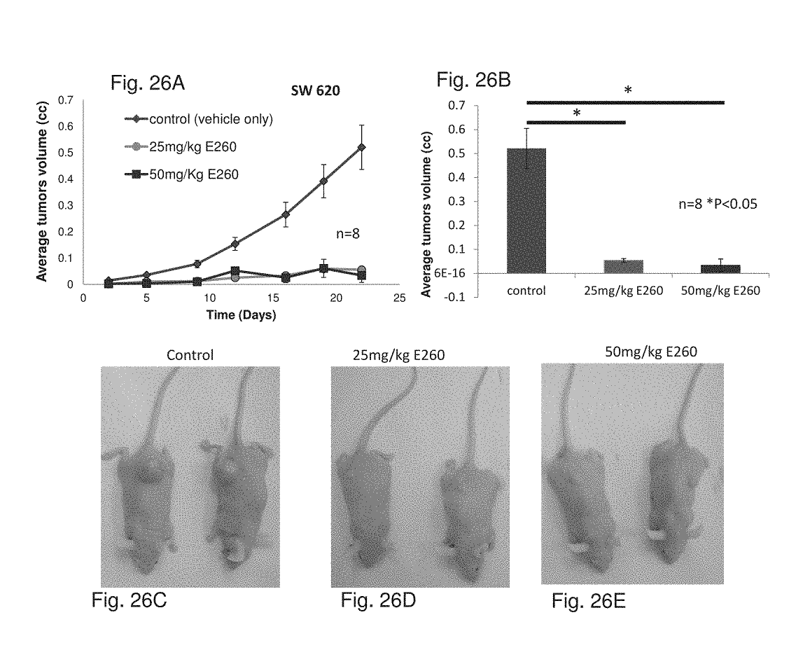

Analogous dose response curves were obtained in PANC-1 (primary tumor derived cells) and su.86.86 (pancreatic cancer cells derived from metastatic site-liver) cells treated with increasing concentrations of 0260 for 24 h, 48 h and 72 h relating to EC50 values (FIGS. 24A-24B). Importantly, Su.86.86 metastatic cells were more sensitive to 0260 than PANC-1 cells derived from a primary tumor, thus supporting the above observations in metastatic colon cancer cells. Further, these results corroborate the previously suggested notion (EXAMPLE 3) that aggressive and metastatic cells are more prone to 0260 cytotoxicity than primary cancer cells.

Taken together, the inventors have demonstrated original findings that Fer regulates PARP activity, as well as cell death processes. Moreover, the invention shows that inhibitors of Fer, specifically the 0260 micellar formulation of the invention, act as selective regulators of PARP, and selectively induce mitochondrial damage, lead to MMP depolarization, autophagy, ATP depletion and necrotic cell death. These findings therefore provide powerful tools for modulation of different cell death mechanisms involved in various disorders.

### Example 9

0260 Suppresses the Progression of CC Xenografts without Toxic Effects In-Vivo

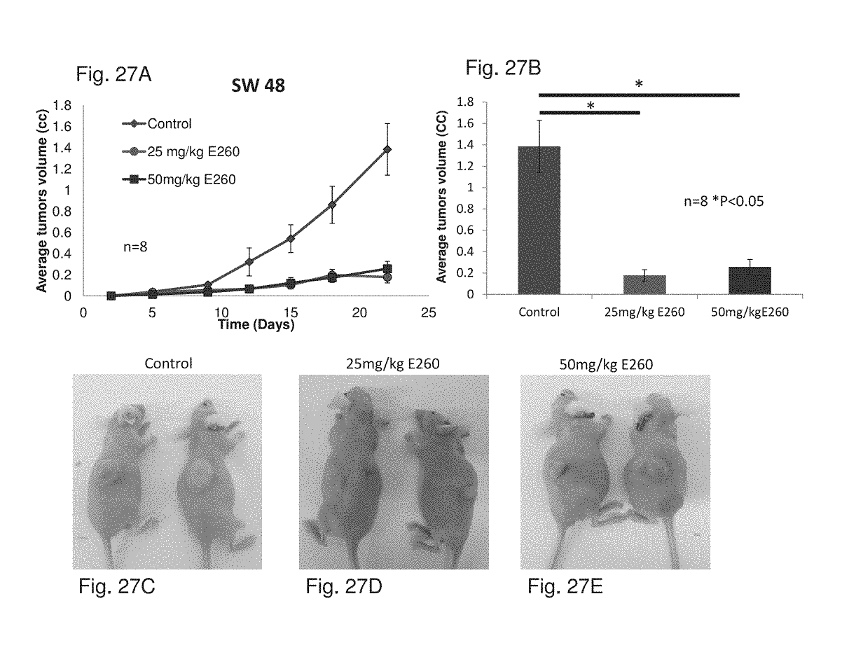

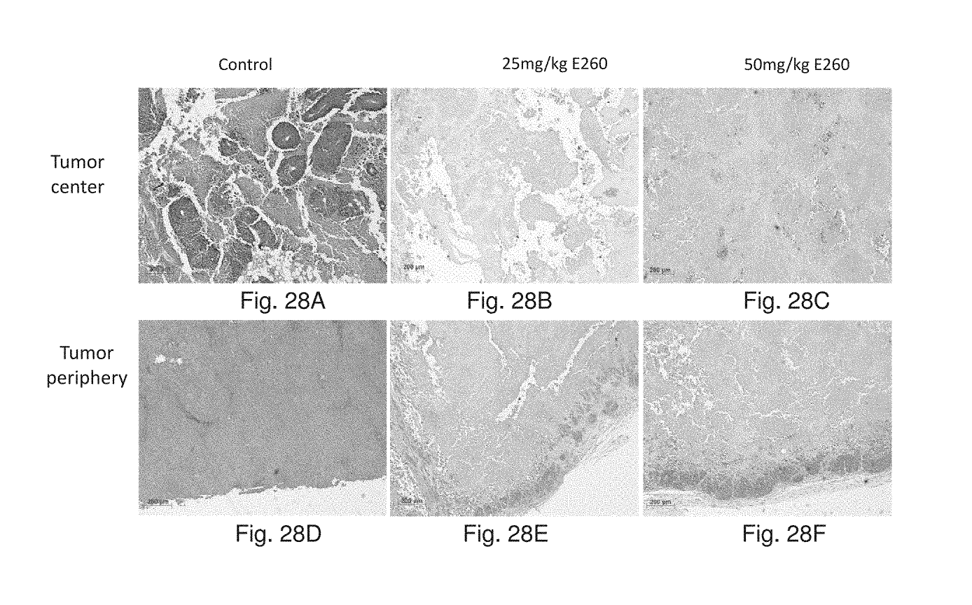

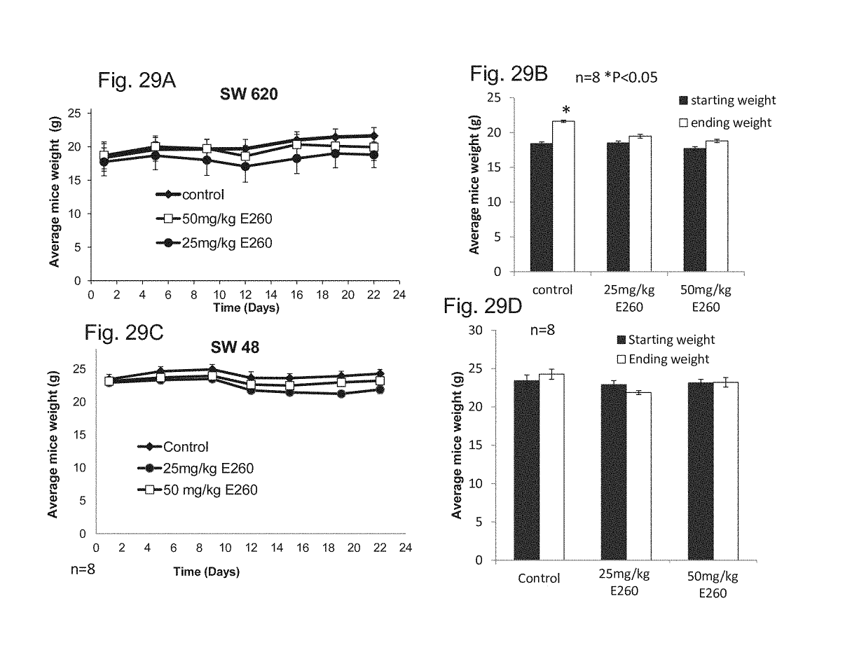

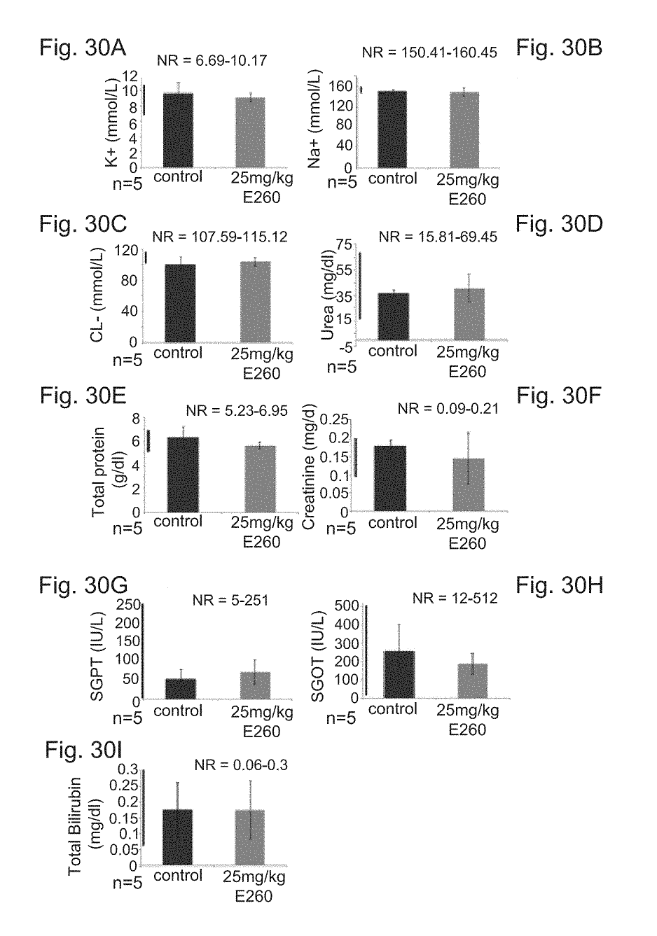

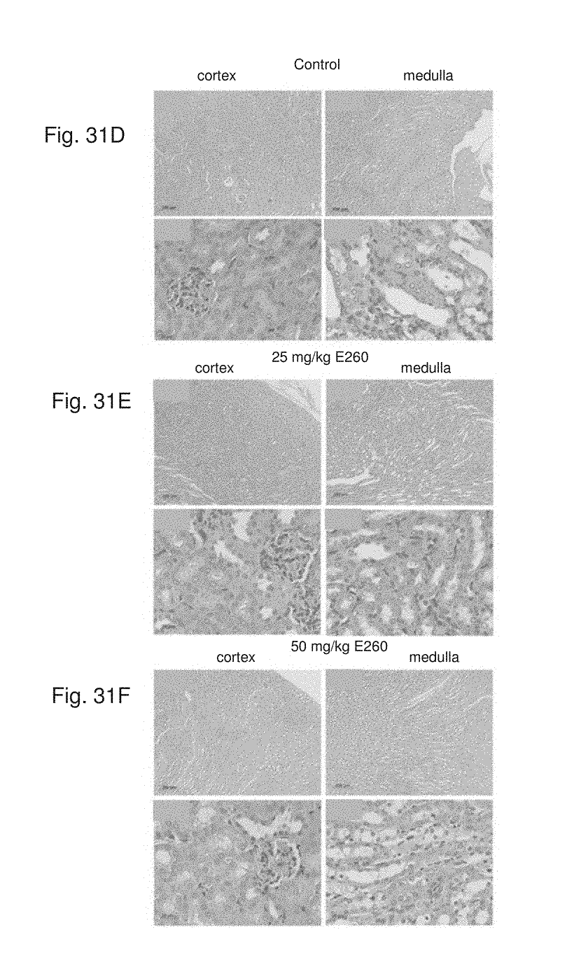

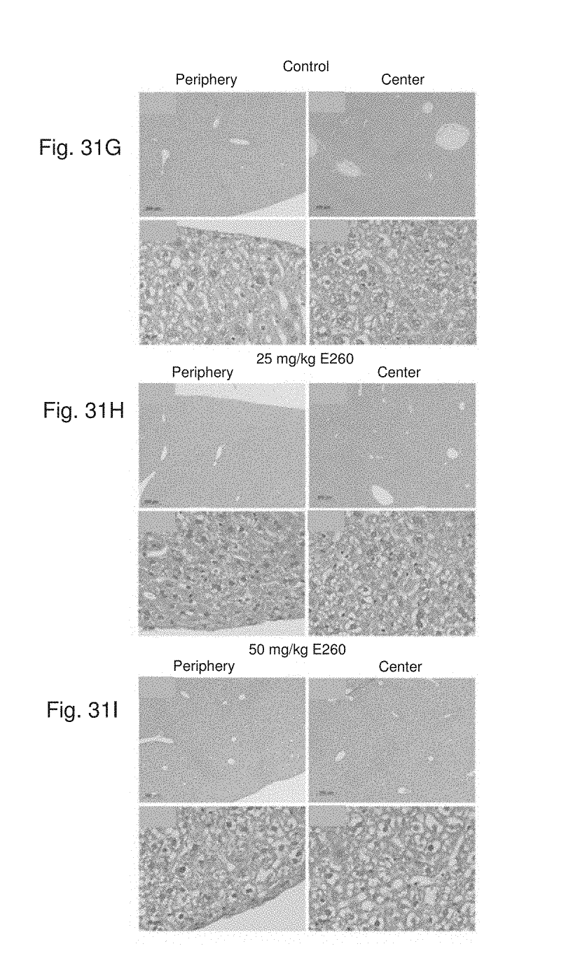

After characterizing the effect of 0260 on cancer cells in-vitro, the inventors turned to study its effect in-vivo. As an initial step, 0260 pharmacokinetic profile was determined in mice. 0260 exhibited a T1/2 of 175 min in the blood, and volume of distribution of 4244 ml/kg suggesting an efficient distribution of the compound in the animal tissues (FIGS. 25A-25B). Based on in-vitro results, to maximize 0260 efficacy in mice xenografts model animals were maintained on a restricted diet by limiting their glucose consumption. To evaluate 0260 efficacy, SW620 xenografts were subcutaneously introduced in immuno-compromised “Nude” mice. Administration of 0260 led to a significant attenuation of tumor progression throughout the experiment, and to 10-fold decrease in average tumor volume after 22 days of treatment (FIGS. 26A-26E). To further demonstrate 0260 anti-cancer activity in-vivo, mice bearing SW48 xenografts were administered with 0260 and tumor progression profiles were determined. 0260 treated mice demonstrated 5- to 6-fold attenuation in tumor progression compared to the control group (FIGS. 27A-27E). Histopathological examination of the SW620 derived tumors dissected at day 22 revealed a highly vascularized and viable tissue at the center and periphery of tumors removed from control treated mice (FIGS. 28A, 28D). In contrast, tumors dissected from 0260 treated mice were comprised mostly of dead necrotic and un-vascularized tissue, both in the tumor center and periphery (FIGS. 28B, 28C, 28E, 28F). Importantly, no significant effect on the treated animal's weight was observed in the in-vivo experiments (FIGS. 29A-29D). Accordingly, daily injection of 0260 did not cause any toxic effects or adverse side effects, as determined by measuring blood markers of liver and kidney functions and blood electrolytes (FIGS. 30A-30I). Histopathological analyses also failed to detect any abnormalities related to toxicity in major organs such as heart (FIGS. 31A-31C), kidneys (FIGS. 31D-31F) and liver (FIGS. 31G-31I).

### Example 10

### 0260 Eliminates Progressive and Total Motility of Sperm Cells

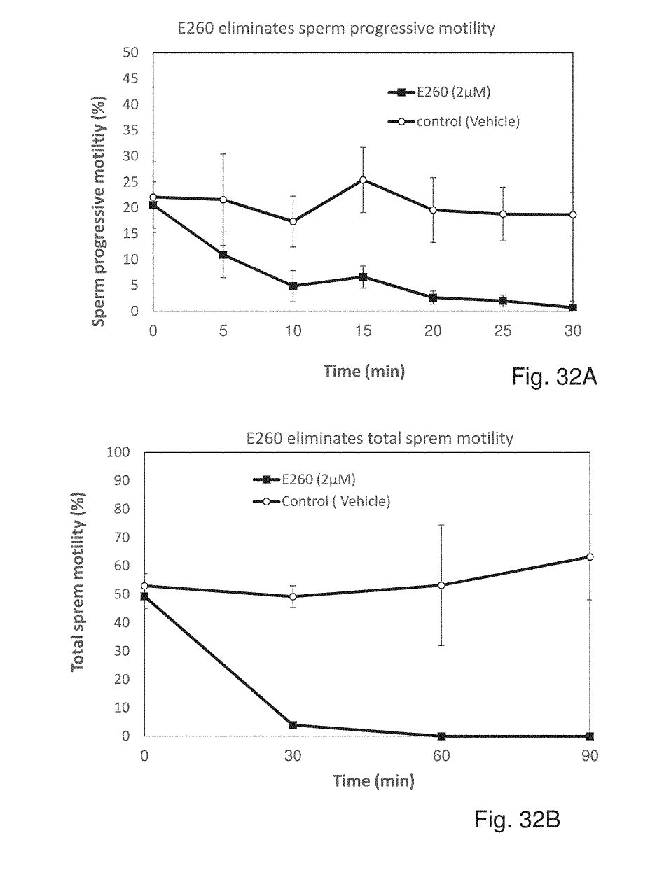

Sperm motility studies were performed using mice sperm cells prepared and extracted as described above, and incubated in the presence of 2 μM 0260 or control solution (vehicle). The percentage of sperm hyper-activated motility (progressive) was carefully calculated by eye observation using light microscope. Sperm cells motility was evaluated using the Computer-Aided Sperm Analysis (CASA) as described above. Percentage of sperm cells exhibiting progressive motility was significantly decreased as early as after 5 min of incubation with 0260 (FIG. 32A). After 30 min both the progressive and the total motility of the sperm cells were practically reduced to zero and total sperm motility (FIGS. 32A-32B). These studies clearly demonstrate significant effect of 0260 on attenuation and even elimination of progressive and total motility of sperm cells. It should be noted that progressive motility is crucial for fertilization of an ova.

### Example 11

High-Throughput Analysis of Phosphorylation Events Resulting from 0260 Exposure (Kinome)

Kinome technique was used for monitoring phosphorylation events and phosphorylation targets of 0260-Fer interaction, using peptide arrays for human targets and phosphorylation-specific antibodies. Construction of peptide arrays relied on information from phosphorylation databases. The following proteins were determined as significant above the threshold of 40% kinase activity inhibition or ATP displacement to the Kinase domain site.

1. CAMK1 (Calcium/calmodulin-dependent protein kinase type 1) being a member of a wide family including 12 kinases, the major part of which have been attributed physiological roles. Notably, only CAMK1 but no other members of this family were significantly inhibited by 0260, suggesting that functional inhibition of CAMK1 in cells may be compensated by other kinases from this family.
2. DAPK1 (Death-associated protein kinase 1) being a positive mediator of gamma-interferon induced cell death and a putative tumor suppressor. Depletion of DAPK1 has been known to result in inhibition of cell growth and xenorgrafts progression in cellular and animal models of triple receptor-negative (TN) breast cancer, from individuals with p53-mutant cancers. Thus, DAPK1 may further serve as a proto-oncogene and a therapeutic target in TN breast cancers.
3. CSF1R (Colony stimulating factor 1 receptor-FMS) being a receptor tyrosine kinase binding to the colony stimulating factor 1 ligand. The CSF1 receptor (CSF1R) is known to regulate macrophages differentiation. Elevated expression of CSF1R has been linked to progression and poor prognosis of numerous malignant tumors. CSF1R drives the recruitment of tumor-associated macrophages (TAMs) to the neoplastic microenvironment of solid tumors and promotes differentiation of TAMS toward a pro-tumorigenic phenotype. Further, over-expression of CSF1R in carcinoma cells drives towards invasiveness and metastasis. Inhibitors of CSF1R are currently being tested in clinical trials as anti-angiogenic agents for cancer therapy. CSF1R is also targeted by Gleevec, and in this context is being tested for anti-inflammatory properties.
4. FLT3 (FMS-like tyrosine kinase 3) being a tyrosine kinase receptor for the FLT3-ligand (FLT3L). It plays a role in the development of hematopoietic-stem cells. FLT3 is a proto-oncogene that is the most commonly duplicated in Acute Myelogenous Leukemia (AML). Inhibitors of FLT3 (Quizartinib and Midostaurin) showed good efficacy and safety in clinical trial for refractory AML, significantly improving overall survival in FLT3-ITD positive AML patients who relapsed after stem cell transplantation, or after failure of salvage chemotherapy.
5. MAPK8 (JNK1) being an intracellular serine/threonine kinase mediating pro-apoptotic signals elicited by cell stimulants like TNF-alpha. JNK1 belongs to the wide family JNKs, of which only JNK1 was found inhibited by 0260, and therefore, as for CAMK1, its inhibition may be compensated by other family members. JNK1/2/3 inhibitors are found in various drug development stages, some of them showed potency in preventing inflammation and cancer progression. Notably, some inhibitors also showed neuroprotective properties following ischemic trauma.
6. MINKI being an intracellular serine/threonine kinase shown to be required for completion of cytokinesis in malignant (Hela) cells. It was also found to be unregulated during activation of the p38 driven-pro-inflammatory pathway. An inhibitor for MINK1 (CGP 57380) has been proven to be efficient in preventing the activation of eIF4E, thereby sensitizing mTORC1 to inhibitors and significantly increasing rapamycin cytotoxic effects towards medulloblastoma tumor cells. Overall the MINK (MNK) family members have been considered as potential targets for cancer therapy and inhibitors these kinases are found in various stages of drug development.

The effect of the 0260-Fer interaction on phosphorylation state of these proteins clearly indicates the involvement of Fer in signaling pathways mediated by the above-identified candidates. Moreover, these results demonstrate the feasibility of using the 0260 Fer inhibitor of the invention as a modulator of malignant, inflammatory and neurodegenerative processes involving signaling pathways mediated by any of MINK1, MAPK8 (JNK1), FLT3 (FMS-like tyrosine kinase 3), CSF1R (Colony stimulating factor 1 receptor-FMS), CAMK1 (Calcium/calmodulin-dependent protein kinase type 1) and DAPK1 (Death-associated protein kinase 1).

1.-53. (canceled)

54. A method for inducing in a cell at least one of, mitochondrial damage and ADP ribosylation activity of poly(ADP-ribose) polymerase 1 (PARP), said mitochondrial damage comprises at least one of mitochondrial membrane potential (MMP) depolarization, deformation of mitochondrial structure/s and down regulation of mitochondrial protein/s, wherein the method comprising the step of contacting said cell with an effective amount of at least one inhibitor of Fer tyrosine kinase (Fer), thereby inducing at least one of mitochondrial damage and enhancing the activity of PARP in said cell.

55. The method according to claim 54, wherein at least one of:
(a) the method is for inducing autophagy in said cell;
(b) the method is for inducing ATP depletion in a cell;
(c) the method is for inducing necrosis in a cell; and
(d) the method is for enhancing ADP ribosylation activity of PARP 1 in a cell.

56. The method according to claim 54, wherein said inhibitor of Fer is a compound of formula I or a pharmaceutically acceptable salt thereof, or any vehicle, matrix, nano- or micro-particle, or composition comprising the same, wherein Formula I has the following structure: 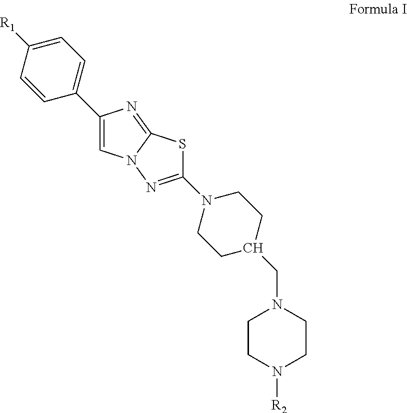
wherein R₁ and R₂ are each independently selected from hydrogen, halogen, C₁₋₆alkyl, C₂₋₆alkenyl, N—(C₁₋₆alkyl)₂ or N—(C₂₋₆alkenyl)₂, wherein the C₁₋₆alkyl and C₂₋₆alkenyl being straight or branched.

57. The method according to claim 56, wherein said compound is 6-(4-isopropyl-phenyl)-2-{4-[(4-methyl-piperazin-1-yl)methyl]piperidin-1-yl}imidazo[2,1-b][1,3,4]thiadiazole.

58. The method according to claim 57, wherein said pharmaceutically acceptable salt is tartarate, said compound is designated herein as E260.

59. The method according to claim 58, wherein said Fer inhibitor is a micellar formulation of compound E260.

60. The method according to claim 54, for inducing in a subject at least one of, specific mitochondrial damage and ADP ribosylation activity of poly (ADP-ribose) polymerase 1 (PARP), said mitochondrial damage comprises at least one of mitochondrial membrane potential (MMP) depolarization, deformation of mitochondrial structure/s and down regulation of mitochondrial protein/s, wherein the method comprising the step of administering to a subject in need thereof an effective amount of at least one inhibitor of Fer tyrosine kinase (Fer), or any vehicle, matrix, nano- or micro-particle, or composition comprising the same, thereby inducing at least one of mitochondrial damage and enhancing the activity of PARP in at least one cell of said subject, wherein at least one of:
(a) the method is for inducing autophagy in at least one cell of said subject;
(b) the method is for inducing ATP depletion in at least one cell of said subject;
(c) the method is for inducing necrosis in at least one cell of said subject; and
(d) the method is for inducing ADP ribosylation activity of PARP-1 in at least one cell of said subject.

61. The method according to claim 60, wherein said inhibitor of Fer is a compound of formula I or a pharmaceutically acceptable salt thereof, or any vehicle, matrix, nano- or micro-particle, or composition comprising the same, wherein Formula I has the following structure: 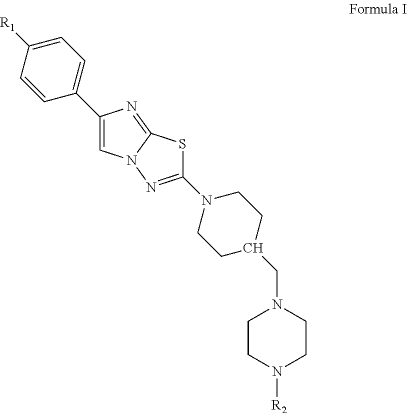
wherein R₁ and R₂ are each independently selected from hydrogen, halogen, C₁₋₆alkyl, C₂₋₆alkenyl, N—(C₁₋₆alkyl)₂ or N—(C₂₋₆alkenyl)₂, wherein the C₁₋₆alkyl and C₂₋₆alkenyl being straight or branched.

62. The method according to claim 60, wherein said subject is suffering from an autophagic diseases or condition, wherein said autophagic diseases or condition is any one of cancer, protein conformational disorder (PCD), a neurodegenerative disease or condition, inflammatory disorder, metabolic disorder or a myopathy.

63. A method for treating, preventing, inhibiting, reducing, ameliorating, eliminating, protecting or delaying the onset of an autophagic diseases or condition or a cancer disease or condition in a subject in need thereof, said method comprising the step of administering to said subject a therapeutically effective amount of at least one inhibitor of Fer or any vehicle, matrix, nano- or micro-particle, or composition comprising the same.

64. The method according to claim 63, wherein said inhibitor of Fer is a compound of formula I or a pharmaceutically acceptable salt thereof, or any vehicle, matrix, nano- or micro-particle, or composition comprising the same, wherein Formula I has the following structure: 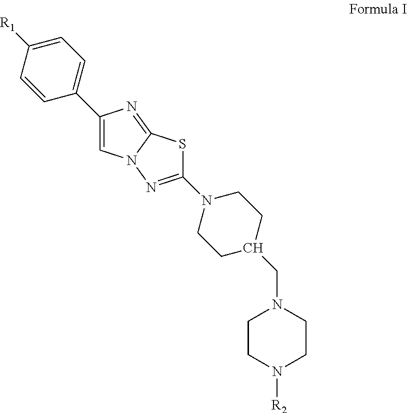
wherein R₁ and R₂ are each independently selected from hydrogen, halogen, C₁₋₆alkyl, C₂₋₆alkenyl, N—(C₁₋₆alkyl)₂ or N—(C₂₋₆alkenyl)₂, wherein the C₁₋₆alkyl and C₂₋₆alkenyl being straight or branched.

65. The method according to claim 64, wherein said compound is 6-(4-isopropyl-phenyl)-2-{4-[(4-methyl-piperazin-1-yl)methyl]piperidin-1-yl}imidazo[2,1-b][1,3,4]thiadiazole.

66. The method according to claim 65, wherein said pharmaceutically acceptable salt is tartarate, said compound is designated herein as E260.

67. The method according to claim 66, wherein said Fer inhibitor is a micellar formulation of compound E260.

68. A micellar formulation comprising a compound or a pharmaceutically acceptable salt thereof of the following structure of Formula I: 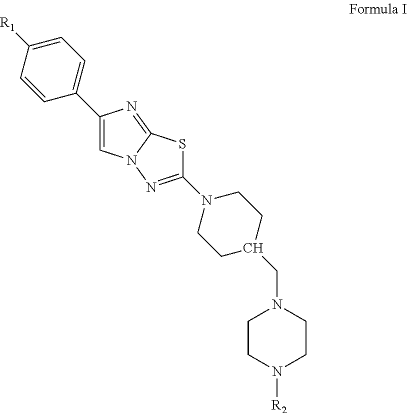
wherein R₁ and R₂ are each independently selected from hydrogen, halogen, C₁₋₆alkyl, C₂₋₆alkenyl, N—(C₁₋₆alkyl)₂ or N—(C₂₋₆alkenyl)₂, wherein the C₁₋₆alkyl and C₂₋₆alkenyl being straight or branched; incorporated in micelles, the micelles formed by mixing a nonionic alcohol alkoxylate surfactant, C₂₋₆alcohol and a buffer selected from acetate, phosphate and sulfate, wherein the diameter of said micelle ranges between about 1 nm to about 100 nm.

69. The micellar formulation according to claim 68, wherein the R₁ is isopropyl and R₂ is methyl.

70. The micellar formulation according to claim 69, wherein the compound is 6-(4-isopropyl-phenyl)-2-{4-[(4-methyl-piperazin-1-yl)methyl]piperidin-1-yl}imidazo[2,1-b][1,3,4]thiadiazole.

71. The micellar formulation according to claim 70, wherein the pharmaceutically acceptable salt is tartarate, said compound is designated herein E260.

72. A composition comprising a micellar formulation according to claim 68 or any vehicle, matrix, nano- or micro-particle thereof, said composition optionally further comprises at least one of pharmaceutically acceptable carrier/s, diluent/s and/or excipient/s.

73. The composition according to claim 72, comprising said micellar formulation in an amount effective for at least one of:
(a) inducing in a cell at least one of, mitochondrial damage and ADP ribosylation activity of poly(ADP-ribose) polymerase 1 (PARP);
(b) inducing in a subject at least one of, specific mitochondrial damage and ADP ribosylation activity of poly(ADP-ribose) polymerase 1 (PARP); and
(c) for inducing mitochondrial damage in sperm cell/s of a subject.

74. The method according to claim 54, for reducing or inhibiting at least one of motility and viability of sperm of a subject, the method comprises the step of administering to said subject or to at least one other subject being in contact with said sperm, an effective amount of at least one inhibitor of Fer or any vehicle, matrix, nano- or micro-particle, or composition comprising the same wherein said inhibitor of Fer is as defined in claim 68.
